SEQUENCE LISTING (440 sequences)

SEQ ID NO: 1 - DNA, 468 bp, Homo sapiens
  CDS (1)..(468)
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 2 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - DNA, 468 bp, Mus musculus
  CDS (1)..(468)
atggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactag

SEQ ID NO: 4 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 5 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 6 - protein, 155 aa, Gorilla gorilla; identical to SEQ ID NO: 2

SEQ ID NO: 7 - protein, 155 aa, Pan troglodytes; identical to SEQ ID NO: 2

SEQ ID NO: 8 - protein, 155 aa, Canis familiaris
MAEGEITTFTALTEKFNLPPGNYMKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 9 - protein, 155 aa, Felis catus
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 10 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLCMDTDGLLYGSQTPNEECLFLERLEENHYVTYISKKHAEKNWFVGLKKNGSVKRGPRTHYGQKAILFLPLPVSSD

SEQ ID NO: 11 - protein, 140 aa, Artificial Sequence
  Synthetic polypeptide
FNLPPGNYKKPKLLYCSNGGHFLRILPDGTVDGTRDRSDQHIQLQLSAESVGEVWIKSTETGQYLAMDTDGLLYGSQTPNEECLFLHRLEENHYNTYISKKHAEKNWFVGLKKNGRVKRGPRTHYGQKAILFLPLPVSSD

SEQ ID NO: 12 - protein, 140 aa, Artificial Sequence
  Synthetic polypeptide
FNLPPGNYKKPVLLYCSNGGHFLRILPDGTVDGTRDRSDQHIQLQLSAESVGEVWIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 13 - protein, 137 aa, Artificial Sequence
  Synthetic polypeptide
MRDSSPLPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 14 - protein, 137 aa, Artificial Sequence
  Synthetic polypeptide
MRDSSPLPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 15 - protein, 137 aa, Artificial Sequence
  Synthetic polypeptide
MRDSSPLPVLLYCSNGGHFLRILPDGTVDGTRDRSDQHIQLQLSAESVGEVYIKSTETGQYLAMDTDGLLYGSQTPNEECLFLVRLEENHYNTYISKKHAEKNWFVGLKKNGSVKRGPRTHYGQKAILFLPLPVSSD

SEQ ID NO: 16 - protein, 127 aa, Artificial Sequence
  Synthetic polypeptide
LYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 17 - protein, 127 aa, Artificial Sequence
  Synthetic polypeptide
LYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSCKRGPRTHYGQKAILFLPLPVSSD

SEQ ID NO: 18 - protein, 127 aa, Artificial Sequence
  Synthetic polypeptide
LYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 19 - protein, 127 aa, Artificial Sequence
  Synthetic polypeptide
LYCSNGGHFLRILPDGTVDGTRDRSDQHIQFQLSAESVGEVYIKSTETGQYLCMDTDGLLYGSQTPNEETLFLERLEENHYVTYISKKHAEKNWFVGLKKNGSVKRGPRTHYGQKAILWLPLPVSSD

SEQ ID NO: 20 - protein, 134 aa, Artificial Sequence
  Synthetic polypeptide
MRDSSPLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 21 - protein, 138 aa, Artificial Sequence
  Synthetic polypeptide
MRDSSPLGGQVLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 22 - protein, 129 aa, Artificial Sequence
  Synthetic polypeptide
VLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 23 - protein, 129 aa, Artificial Sequence
  Synthetic polypeptide
VLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSCKRGPRTHYGQKAILFLPLPVSSD

SEQ ID NO: 24 - protein, 129 aa, Artificial Sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 25 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYIAKEAAEKNAFVGLKKNGSCKRGPRTHYGQKAILFLPLPVSSD

SEQ ID NO: 26 - protein, 137 aa, Artificial Sequence
  Synthetic polypeptide
PPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 27 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 28 - protein, 127 aa, Artificial Sequence
  Synthetic polypeptide
LYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 29 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 30 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 31 - protein, 140 aa, Artificial Sequence
  Synthetic polypeptide
FNLPPGNYKKPVLLYCSNGGHFLRILPDGTVDGTRDRSDQHIQLQVSAESVGEVYIKSTETGQYLAMDTDGLLYGSQTPNEECLFLVRLEENHYVTYISKKHAEKNWFVGLKKNGSCKRGPRTHYGQKAILFLVLPVSSD

SEQ ID NO: 32 - protein, 134 aa, Artificial Sequence
  Synthetic polypeptide
NYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 33 - protein, 134 aa, Artificial Sequence
  Synthetic polypeptide
NYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCERGPRTHYGQKAILFLPLPVSSD

SEQ ID NO: 34 - protein, 135 aa, Artificial Sequence
  Synthetic polypeptide
GGQVKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 35 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 36 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 37 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 38 - protein, 129 aa, Artificial Sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 39 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 40 - protein, 129 aa, Artificial Sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 41 - protein, 140 aa, Artificial Sequence
  Synthetic polypeptide
FNLPPGNYKKPKLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 42 - protein, 140 aa, Artificial Sequence
  Synthetic polypeptide
FNLPPGNYKKPKLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 43 - protein, 140 aa, Artificial Sequence
  Synthetic polypeptide
FNLPPGNYKKPVLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 44 - protein, 140 aa, Artificial Sequence
  Synthetic polypeptide
FNLPPGNYKKPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 45 - protein, 131 aa, Artificial Sequence
  Synthetic polypeptide
KPKLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 46 - protein, 129 aa, Artificial Sequence
  Synthetic polypeptide
KLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 47 - protein, 131 aa, Artificial Sequence
  Synthetic polypeptide
KPKLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 48 - protein, 129 aa, Artificial Sequence
  Synthetic polypeptide
KLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 49 - protein, 131 aa, Artificial Sequence
  Synthetic polypeptide
KPVLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 50 - protein, 129 aa, Artificial Sequence
  Synthetic polypeptide
VLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 51 - protein, 131 aa, Artificial Sequence
  Synthetic polypeptide
KPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 52 - protein, 129 aa, Artificial Sequence
  Synthetic polypeptide
VLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 53 - protein, 134 aa, Artificial Sequence
  Synthetic polypeptide
FNLPPGNYKKPKLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 54 - protein, 134 aa, Artificial Sequence
  Synthetic polypeptide
FNLPPGNYKKPVLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 55 - protein, 134 aa, Artificial Sequence
  Synthetic polypeptide
FNLPPGNYKKPVLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 56 - protein, 134 aa, Artificial Sequence; identical to SEQ ID NO: 55
  Synthetic polypeptide

SEQ ID NO: 57 - protein, 125 aa, Artificial Sequence
  Synthetic polypeptide
KPKLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 58 - protein, 123 aa, Artificial Sequence
  Synthetic polypeptide
KLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 59 - protein, 125 aa, Artificial Sequence
  Synthetic polypeptide
KPVLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 60 - protein, 123 aa, Artificial Sequence
  Synthetic polypeptide
VLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 61 - protein, 125 aa, Artificial Sequence
  Synthetic polypeptide
KPKLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 62 - protein, 123 aa, Artificial Sequence
  Synthetic polypeptide
KLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 63 - protein, 125 aa, Artificial Sequence
  Synthetic polypeptide
VPKLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 64 - protein, 123 aa, Artificial Sequence
  Synthetic polypeptide
VLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 65 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCNRGPRTHYGQKAILFLPLPVSSD

SEQ ID NO: 66 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCERGPRTHYGQKAILFLPLPVSSD

SEQ ID NO: 67 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 68 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVNRGPRTHYGQKAILFLPLPVSSD

SEQ ID NO: 69 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVERGPRTHYGQKAILFLPLPVSSD

SEQ ID NO: 70 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 71 - protein, 129 aa, Artificial Sequence; identical to SEQ ID NO: 24
  Synthetic polypeptide

SEQ ID NO: 72 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 73 - protein, 129 aa, Artificial Sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 74 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 75 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 76 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCNRGPRTHYGQKAILFLPLPVSSD

SEQ ID NO: 77 - protein, 129 aa, Artificial Sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCERGPRTHYGQKAILFLPLPVSSD

SEQ ID NO: 78 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 79 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCERGPRTHYGQKAILFLPLPVSSD

SEQ ID NO: 80 - protein, 140 aa, Artificial Sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 81 - protein, 140 aa, Artificial Sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 82 - protein, 140 aa, Artificial Sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 83 - protein, 140 aa, Artificial Sequence
  Synthetic polypeptide
FNLPPGDQDQNQLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 84 - protein, 131 aa, Artificial Sequence
  Synthetic polypeptide
KPKLLYCSNGGHFLRILPDGTVDGTRDRSDPHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 85 - protein, 129 aa, Artificial Sequence
  Synthetic polypeptide
KLLYCSNGGHFLRILPDGTVDGTRDRSDPHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 86 - protein, 140 aa, Artificial Sequence
  Synthetic polypeptide
FNLPPGNYKKPVLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 87 - protein, 131 aa, Artificial Sequence
  Synthetic polypeptide
KPVLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 88 - protein, 129 aa, Artificial Sequence
  Synthetic polypeptide
VLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 89 - protein, 131 aa, Artificial Sequence
  Synthetic polypeptide
KPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 90 - protein, 129 aa, Artificial Sequence
  Synthetic polypeptide
KLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 91 - protein, 140 aa, Artificial Sequence
  Synthetic polypeptide
FNLPPGNYKKPVLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 92 - protein, 131 aa, Artificial Sequence
  Synthetic polypeptide
KPVLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 93 - protein, 129 aa, Artificial Sequence; identical to SEQ ID NO: 22
  Synthetic polypeptide

SEQ ID NO: 94 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSPVRGPRTHYGQKAILFLPLPVSSD

SEQ ID NO: 95 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSPVRGPRTHYGQKAILFLPLPVSSD

SEQ ID NO: 96 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSPVRGPRTHYGQKAILFLPLPVSSD

SEQ ID NO: 97 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 98 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 99 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 100 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 101 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 102 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 103 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 104 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLDQNGSCVRGPRTHYGQKAILFLPLPVSSD

SEQ ID NO: 105 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

SEQ ID NO: 106 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 107 - protein, 140 aa, Artificial Sequence
  Synthetic polypeptide
FNLPPGNYKKPVLLYCSNGGHFLRILPDGTVDGTEDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 108 - protein, 131 aa, Artificial Sequence
  Synthetic polypeptide
KPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 109 - protein, 131 aa, Artificial Sequence
  Synthetic polypeptide
KPVLLYCSNGGHFLRILPDGTVDGTEDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

SEQ ID NO: 110 - protein, 140 aa, Artificial Sequence
  Synthetic polypeptide
FNLPPGNYKKPVLLYCSNGGHFLRILPDGTVDGTEDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

SEQ ID NO: 111 - protein, 131 aa, Artificial Sequence
  Synthetic polypeptide
KPVLLYCSNGGHFLRILPDGTVDGTEDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 112 - protein, 131 aa, Artificial Sequence
  Synthetic polypeptide
KPVLLYCSNGGHFLRILPDGTVDGTEDRSDQHIQFQLSAESVGEVYIKSTETGQYLAMDTDGLLYGSQTPNEETLFLERLEENHYVTYISKKHAEKNWFVGLDQNGSVVRGPRTHYGQKAILWLPLPVSSD

SEQ ID NO: 113 - protein, 140 aa, Artificial Sequence
  Synthetic polypeptide
FNLPPGNYKKPKLLYCSNGGHFLRILPDGTVDGTVDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 114 - protein, 140 aa, Artificial Sequence
  Synthetic polypeptide
FNLPPGNYKKPKLLYCSNGGHFLRILPDGTVDGTVDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 115 - protein, 140 aa, Artificial Sequence
  Synthetic polypeptide
FNLPPGNYKKPVLLYCSNGGHFLRILPDGTVDGTVDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 116 - protein, 131 aa, Artificial Sequence
  Synthetic polypeptide
KPKLLYCSNGGHFLRILPDGTVDGTVDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 117 - protein, 131 aa, Artificial Sequence
  Synthetic polypeptide
KPVLLYCSNGGHFLRILPDGTVDGTVDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

SEQ ID NO: 118 - protein, 140 aa, Artificial Sequence
  Synthetic polypeptide
FNLPPGNYKKPVLLYCSNGGHFLRILPDGTVDGTVDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

SEQ ID NO: 119 - protein, 131 aa, Artificial Sequence
  Synthetic polypeptide
KPVLLYCSNGGHFLRILPDGTVDGTVDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 120 - protein, 131 aa, Artificial Sequence
  Synthetic polypeptide
KPVLLYCSNGGHFLRILPDGTVDGTVDRSDQHIQFQLSAESVGEVYIKSTETGQYLAMDTDGLLYGSQTPNEETLFLERLEENHYVTYISKKHAEKNWFVGLDQNGSVVRGPRTHYGQKAILWLPLPVSSD

SEQ ID NO: 121 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVVGPRTHYGQKAILFLPLPVSSD

SEQ ID NO: 122 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 123 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLVRLEENHYNTYISKKHAEKNWFVGLKKNGSVKRGPRTHYGQKAILFLPLPVSSD

SEQ ID NO: 124 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLVRLEENHVNTYISKKHAEKNWFVGLKKNGSVKRGPRTHYGQKAILFLPLPVSSD

SEQ ID NO: 125 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVVTYISKKHAEKNWFVGLKKNGSVKRGPRTHYGQKAILFLPLPVSSD

SEQ ID NO: 126 - protein, 140 aa, Artificial Sequence
  Synthetic polypeptide
FNLPPGNYKKPVLLYCSNGGHFLRILPDGTVDGTRDRSDQHIQLQVSAESVGEVYIKSTETGQYLAMDTDGLLYGSQTPNEECLFLVRLEENHYVTYISKKHAEKNWFVGLKKNGSVKRGPRTHYGQKAILFLVLPVSSD

SEQ ID NO: 127 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVVRGPRTHYGQKAILFLPLPVSSD

SEQ ID NO: 128 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEETLFLERLEENHYVTYISKKHAEKNWFVGLKKNGSVKRGPRTHYGQKAILFLPLPVSSD

SEQ ID NO: 129 - protein, 131 aa, Artificial Sequence
  Synthetic polypeptide
KPVLLYTSNGGHFLRILPDGTVDGTRDRSDQHIQLQLSAESVGEVYIKSTETGQYLAMDTDGLLYGSQTPNEESLFLERLEENHYVTYISKKHAEKNWFVGLKKNGSAKRGPRTHYGQKAILFLPLPVSSD

SEQ ID NO: 130 - protein, 131 aa, Artificial Sequence
  Synthetic polypeptide
KPKLLYCSNGGYFLRILPDGTVDGTRDRSDQHIQFQLSAESVGEVYIKSTETGQYLAMDTDGLLYGSQTPNEECLFLERLEENHYNTYISKKYAEKNWYVGLKKNGSVKRGPRTHYGQKAILFLPLPVSSD

SEQ ID NO: 131 - protein, 131 aa, Artificial Sequence
  Synthetic polypeptide
KPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 132 - protein, 140 aa, Artificial Sequence
  Synthetic polypeptide
FNLPPGNYKKPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 133 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 134 - protein, 140 aa, Artificial Sequence; identical to SEQ ID NO: 98
  Synthetic polypeptide

SEQ ID NO: 135 - protein, 140 aa, Artificial Sequence; identical to SEQ ID NO: 91
  Synthetic polypeptide

SEQ ID NO: 136 - protein, 131 aa, Artificial Sequence; identical to SEQ ID NO: 92
  Synthetic polypeptide

SEQ ID NO: 137 - protein, 140 aa, Artificial Sequence; identical to SEQ ID NO: 44
  Synthetic polypeptide

SEQ ID NO: 138 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 139 - protein, 140 aa, Artificial Sequence; identical to SEQ ID NO: 105
  Synthetic polypeptide

SEQ ID NO: 140 - protein, 131 aa, Artificial Sequence; identical to SEQ ID NO: 51
  Synthetic polypeptide

SEQ ID NO: 141 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 142 - protein, 140 aa, Artificial Sequence; identical to SEQ ID NO: 122
  Synthetic polypeptide

SEQ ID NO: 143 - protein, 140 aa, Artificial Sequence; identical to SEQ ID NO: 123
  Synthetic polypeptide

SEQ ID NO: 144 - protein, 140 aa, Artificial Sequence; identical to SEQ ID NO: 121
  Synthetic polypeptide

SEQ ID NO: 145 - protein, 140 aa, Artificial Sequence
  Synthetic polypeptide
FNLPPGNYKKPVLLYCSNGGHFLRILPDGTVDGTEDRSDQHIQLQLSAESVGEVYIKSTETGQYLAMDTDFNLPPGNYKKPVLLYCSNGGHFLRILPDGTVDGTEDRSDQHIQLQLSAESVGEVYIKSTETGQYLAMDTD

SEQ ID NO: 146 - protein, 131 aa, Artificial Sequence
  Synthetic polypeptide
KPVLLYCSNGGHFLRILPDGTVDGTEDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 147 - protein, 140 aa, Artificial Sequence
  Synthetic polypeptide
FNLPPGNYKKPKLLYCSNGGYFLRILPDGTVDGTRDRSDQHIQFQLSAESVGEVYIKSTETGQYLAMDTDGLLYGSQTPNEECLFLERLEENHYNTYISKKYAEKNWYVGLKKNGSCKRGPRTHYGQKAILFLPLPVSSD

SEQ ID NO: 148 - protein, 135 aa, Artificial Sequence
  Synthetic polypeptide
KPKLLYCSNGGYFLRILPDGTVDGTRDRSDQHIQFQLSAESVGEVYIKSTETGQYLAMDTDHLHFGLLYGSQTPNEECLFLERLEENHYNTYISKKYAEKNWYVGLKKNGSCKRGPRTHYGQKAILFLPLPVSSD

SEQ ID NO: 149 - protein, 140 aa, Artificial Sequence
  Synthetic polypeptide
FNLPPGNYKKPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSCKRGPRTHYGQKAILFLPLPVSSD

SEQ ID NO: 150 - protein, 131 aa, Artificial Sequence; identical to SEQ ID NO: 131
  Synthetic polypeptide

SEQ ID NO: 151 - protein, 140 aa, Artificial Sequence
  Synthetic polypeptide
FNLPPGNYKKPKLLYCSNGGYFLRILPDGTVDGTRDRSDQHIQFQLSAESVGEVYIKSTETGQYLAMDTDGLLYGSQTPNEECLFLERLEENHYNTYISKKYAEKNWYVGLKKNGSVKRGPRTHYGQKAILFLPLPVSSD

SEQ ID NO: 152 - protein, 140 aa, Artificial Sequence
  Synthetic polypeptide
FNLPPGNYKKPKLLYCSNGGYFLRILPDGTVDGTRDRSDQHIQFQLSAESVGEVYIKSTETGQYLCMDTDGLLYGSQTPNEECLFLERLEENHYNTYISKKYAEKNWYVGLKKNGSVKRGPRTHYGQKAILFLPLPVSSD

SEQ ID NO: 153 - protein, 140 aa, Artificial Sequence
  Synthetic polypeptide
FNLPPGNYKKPKLLYCSNGGYFLRILPDGTVDGTEDRSDQHIQFQLSAESVGEVYIKSTETGQYLAMDTDGLLYGSQTPNEECLFLERLEENHYNTYISKKYAEKNWYVGLKKNGSVKRGPRTHYGQKAILFLPLPVSSD

SEQ ID NO: 154 - protein, 140 aa, Artificial Sequence
  Synthetic polypeptide
FNLPPGNYKKPVLLYCSNGGYFLRILPDGTVDGTRDRSDQHIQFQLSAESVGEVYIKSTETGQYLAMDTDGLLYGSQTPNEECLFLERLEENHVNTYISKKYAEKNWYVGLKKNGSVKRGPRTHYGQKAILFLPLPVSSD

SEQ ID NO: 155 - protein, 140 aa, Artificial Sequence
  Synthetic polypeptide
FNLPPGNYKKPKLLYCSRGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 156 - protein, 131 aa, Artificial Sequence
  Synthetic polypeptide
KPKLLYCSRGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 157 - protein, 140 aa, Artificial Sequence
  Synthetic polypeptide
FNLPPGNYKKPVLLYCSRGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 158 - protein, 131 aa, Artificial Sequence
  Synthetic polypeptide
KPVLLYCSRGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 159 - protein, 140 aa, Artificial Sequence
  Synthetic polypeptide
FNLPPGNYKKPKLLYCSRGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 160 - protein, 140 aa, Artificial Sequence
  Synthetic polypeptide
FNLPPGNYKKPKLLYCSRGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 161 - protein, 140 aa, Artificial Sequence
  Synthetic polypeptide
FNLPPGNYKKPKLLYCSRGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 162 - protein, 140 aa, Artificial Sequence
  Synthetic polypeptide
FNLPPGNYKKPVLLYCSRGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 163 - protein, 140 aa, Artificial Sequence
  Synthetic polypeptide
FNLPPGNYKKPKLLYCSKGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 164 - protein, 131 aa, Artificial Sequence
  Synthetic polypeptide
KPKLLYCSKGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 165 - protein, 140 aa, Artificial Sequence
  Synthetic polypeptide
FNLPPGNYKKPVLLYCSKGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 166 - protein, 131 aa, Artificial Sequence
  Synthetic polypeptide
KPVLLYCSKGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 167 - protein, 140 aa, Artificial Sequence
  Synthetic polypeptide
FNLPPGNYKKPKLLYCSKGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 168 - protein, 140 aa, Artificial Sequence
  Synthetic polypeptide
FNLPPGNYKKPKLLYCSKGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 169 - protein, 140 aa, Artificial Sequence
  Synthetic polypeptide
FNLPPGNYKKPKLLYCSKGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 170 - protein, 140 aa, Artificial Sequence
  Synthetic polypeptide
FNLPPGNYKKPVLLYCSKGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 171 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKRGSCKRGPRTHYGQKAILFLPLPVSSD

SEQ ID NO: 172 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKRGSCKRGPRTHYGQKAILFLPLPVSSD

SEQ ID NO: 173 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKRGSCKRGPRTHYGQKAILFLPLPVSSD

SEQ ID NO: 174 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKRGSVKRGPRTHYGQKAILFLPLPVSSD

SEQ ID NO: 175 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKRGSVKRGPRTHYGQKAILFLPLPVSSD

SEQ ID NO: 176 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKRGSCKRGPRTHYGQKAILFLPLPVSSD

SEQ ID NO: 177 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKRGSVKRGPRTHYGQKAILFLPLPVSSD

SEQ ID NO: 178 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKRGSVKRGPRTHYGQKAILFLPLPVSSD

SEQ ID NO: 179 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKKGSCKRGPRTHYGQKAILFLPLPVSSD

SEQ ID NO: 180 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKKGSCKRGPRTHYGQKAILFLPLPVSSD

SEQ ID NO: 181 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKKGSCKRGPRTHYGQKAILFLPLPVSSD

SEQ ID NO: 182 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKKGSVKRGPRTHYGQKAILFLPLPVSSD

SEQ ID NO: 183 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKKGSVKRGPRTHYGQKAILFLPLPVSSD

SEQ ID NO: 184 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKKGSCKRGPRTHYGQKAILFLPLPVSSD

SEQ ID NO: 185 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKKGSVKRGPRTHYGQKAILFLPLPVSSD

SEQ ID NO: 186 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKKGSVKRGPRTHYGQKAILFLPLPVSSD

SEQ ID NO: 187 - protein, 140 aa, Artificial Sequence
  Synthetic polypeptide
FNLPPGNYKKPKLLYCRNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 188 - protein, 131 aa, Artificial Sequence
  Synthetic polypeptide
KPKLLYCRNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 189 - protein, 140 aa, Artificial Sequence
  Synthetic polypeptide
FNLPPGNYKKPVLLYCRNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 190 - protein, 131 aa, Artificial Sequence
  Synthetic polypeptide
KPVLLYCRNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 191 - protein, 140 aa, Artificial Sequence
  Synthetic polypeptide
FNLPPGNYKKPKLLYCRNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 192 - protein, 140 aa, Artificial Sequence
  Synthetic polypeptide
FNLPPGNYKKPKLLYCRNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 193 - protein, 140 aa, Artificial Sequence
  Synthetic polypeptide
FNLPPGNYKKPKLLYCRNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 194 - protein, 140 aa, Artificial Sequence
  Synthetic polypeptide
FNLPPGNYKKPVLLYCRNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 195 - protein, 140 aa, Artificial Sequence
  Synthetic polypeptide
FNLPPGNYKKPKLLYCKNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 196 - protein, 131 aa, Artificial Sequence
  Synthetic polypeptide
KPKLLYCKNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 197 - protein, 140 aa, Artificial Sequence
  Synthetic polypeptide
FNLPPGNYKKPVLLYCKNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 198 - protein, 131 aa, Artificial Sequence
  Synthetic polypeptide
KPVLLYCKNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 199 - protein, 140 aa, Artificial Sequence
  Synthetic polypeptide
FNLPPGNYKKPKLLYCKNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 200 - protein, 140 aa, Artificial Sequence
  Synthetic polypeptide
FNLPPGNYKKPKLLYCKNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 201 - protein, 140 aa, Artificial Sequence
  Synthetic polypeptide
FNLPPGNYKKPKLLYCKNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 202 - protein, 140 aa, Artificial Sequence
  Synthetic polypeptide
FNLPPGNYKKPVLLYCKNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 203 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGRKAILFLPLPVSSD

SEQ ID NO: 204 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGRKAILFLPLPVSSD

SEQ ID NO: 205 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGRKAILFLPLPVSSD

SEQ ID NO: 206 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGRKAILFLPLPVSSD

SEQ ID NO: 207 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGRKAILFLPLPVSSD

SEQ ID NO: 208 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSCKRGPRTHYGRKAILFLPLPVSSD

SEQ ID NO: 209 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGRKAILFLPLPVSSD

SEQ ID NO: 210 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGRKAILFLPLPVSSD

SEQ ID NO: 211 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGKKAILFLPLPVSSD

SEQ ID NO: 212 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGKKAILFLPLPVSSD

SEQ ID NO: 213 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGKKAILFLPLPVSSD

SEQ ID NO: 214 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGKKAILFLPLPVSSD

SEQ ID NO: 215 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGKKAILFLPLPVSSD

SEQ ID NO: 216 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSCKRGPRTHYGKKAILFLPLPVSSD

SEQ ID NO: 217 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGKKAILFLPLPVSSD

SEQ ID NO: 218 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGKKAILFLPLPVSSD

SEQ ID NO: 219 - protein, 140 aa, Artificial Sequence
  Synthetic polypeptide
FNLPPGNYKKPKLLYCSNGGHFLRILPDGTVDGTRDRSDQHIQLQLSADSVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 220 - protein, 131 aa, Artificial Sequence
  Synthetic polypeptide
KPKLLYCSNGGHFLRILPDGTVDGTRDRSDQHIQLQLSADSVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 221 - protein, 140 aa, Artificial Sequence
  Synthetic polypeptide
FNLPPGNYKKPVLLYCSNGGHFLRILPDGTVDGTRDRSDQHIQLQLSADSVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 222 - protein, 131 aa, Artificial Sequence
  Synthetic polypeptide
KPVLLYCSNGGHFLRILPDGTVDGTRDRSDQHIQLQLSADSVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 223 - protein, 140 aa, Artificial Sequence
  Synthetic polypeptide
FNLPPGNYKKPKLLYCSNGGHFLRILPDGTVDGTRDRSDQHIQLQLSADSVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 224 - protein, 140 aa, Artificial Sequence
  Synthetic polypeptide
FNLPPGNYKKPKLLYCSNGGHFLRILPDGTVDGTRDRSDQHIQLQLSADSVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 225 - protein, 140 aa, Artificial Sequence
  Synthetic polypeptide
FNLPPGNYKKPKLLYCSNGGHFLRILPDGTVDGTEDRSDQHIQLQLSADSVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 226 - protein, 140 aa, Artificial Sequence
  Synthetic polypeptide
FNLPPGNYKKPVLLYCSNGGHFLRILPDGTVDGTRDRSDQHIQLQLSADSVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 227 - protein, 140 aa, Artificial Sequence
  Synthetic polypeptide
FNLPPGNYKKPKLLYCSNGGHFLRILPDGTVDGTRDRSDQHIQLQLSAKSVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 228 - protein, 131 aa, Artificial Sequence
  Synthetic polypeptide
KPKLLYCSNGGHFLRILPDGTVDGTRDRSDQHIQLQLSAKSVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 229 - protein, 140 aa, Artificial Sequence
  Synthetic polypeptide
FNLPPGNYKKPVLLYCSNGGHFLRILPDGTVDGTRDRSDQHIQLQLSAKSVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 230 - protein, 131 aa, Artificial Sequence
  Synthetic polypeptide
KPVLLYCSNGGHFLRILPDGTVDGTRDRSDQHIQLQLSAKSVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 231 - protein, 140 aa, Artificial Sequence
  Synthetic polypeptide
FNLPPGNYKKPKLLYCSNGGHFLRILPDGTVDGTRDRSDQHIQLQLSAKSVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 232 - protein, 140 aa, Artificial Sequence
  Synthetic polypeptide
FNLPPGNYKKPKLLYCSNGGHFLRILPDGTVDGTRDRSDQHIQLQLSAKSVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 233 - protein, 140 aa, Artificial Sequence
  Synthetic polypeptide
FNLPPGNYKKPKLLYCSNGGHFLRILPDGTVDGTEDRSDQHIQLQLSAKSVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 234 - protein, 140 aa, Artificial Sequence
  Synthetic polypeptide
FNLPPGNYKKPVLLYCSNGGHFLRILPDGTVDGTRDRSDQHIQLQLSAKSVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 235 - protein, 140 aa, Artificial Sequence
  Synthetic polypeptide
FNLPPGNYKKPKLLYCSNGGHFLRILPDGTVDGTRDRSDQHIQLQLSAESVGEVFIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 236 - protein, 131 aa, Artificial Sequence
  Synthetic polypeptide
KPKLLYCSNGGHFLRILPDGTVDGTRDRSDQHIQLQLSAESVGEVFIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 237 - protein, 140 aa, Artificial Sequence
  Synthetic polypeptide
FNLPPGNYKKPVLLYCSNGGHFLRILPDGTVDGTRDRSDQHIQLQLSAESVGEVFIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 238 - protein, 131 aa, Artificial Sequence
  Synthetic polypeptide
KPVLLYCSNGGHFLRILPDGTVDGTRDRSDQHIQLQLSAESVGEVFIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 239 - protein, 140 aa, Artificial Sequence
  Synthetic polypeptide
FNLPPGNYKKPKLLYCSNGGHFLRILPDGTVDGTRDRSDQHIQLQLSAESVGEVFIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 240 - protein, 140 aa, Artificial Sequence
  Synthetic polypeptide
FNLPPGNYKKPKLLYCSNGGHFLRILPDGTVDGTRDRSDQHIQLQLSAESVGEVFIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 241 - protein, 140 aa, Artificial Sequence
  Synthetic polypeptide
FNLPPGNYKKPKLLYCSNGGHFLRILPDGTVDGTEDRSDQHIQLQLSAESVGEVFIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 242 - protein, 140 aa, Artificial Sequence
  Synthetic polypeptide
FNLPPGNYKKPVLLYCSNGGHFLRILPDGTVDGTRDRSDQHIQLQLSAESVGEVFIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 243 - protein, 140 aa, Artificial Sequence
  Synthetic polypeptide
FNLPPGNYKKPKLLYCSNGGHFLRILPDGTVDGTRDRSDQHIQLQLSAESVGEVVIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 244 - protein, 131 aa, Artificial Sequence
  Synthetic polypeptide
KPKLLYCSNGGHFLRILPDGTVDGTRDRSDQHIQLQLSAESVGEVVIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 245 - protein, 140 aa, Artificial Sequence
  Synthetic polypeptide
FNLPPGNYKKPVLLYCSNGGHFLRILPDGTVDGTRDRSDQHIQLQLSAESVGEVVIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 246 - protein, 131 aa, Artificial Sequence
  Synthetic polypeptide
KPVLLYCSNGGHFLRILPDGTVDGTRDRSDQHIQLQLSAESVGEVVIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 247 - protein, 140 aa, Artificial Sequence
  Synthetic polypeptide
FNLPPGNYKKPKLLYCSNGGHFLRILPDGTVDGTRDRSDQHIQLQLSAESVGEVVIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 248 - protein, 140 aa, Artificial Sequence
  Synthetic polypeptide
FNLPPGNYKKPKLLYCSNGGHFLRILPDGTVDGTRDRSDQHIQLQLSAESVGEVVIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 249 - protein, 140 aa, Artificial Sequence
  Synthetic polypeptide
FNLPPGNYKKPKLLYCSNGGHFLRILPDGTVDGTEDRSDQHIQLQLSAESVGEVVIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 250 - protein, 140 aa, Artificial Sequence
  Synthetic polypeptide
FNLPPGNYKKPVLLYCSNGGHFLRILPDGTVDGTRDRSDQHIQLQLSAESVGEVVIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 251 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLELLEENHYNTYISKKHAEKNWFVGLKKNGSCKRGPRTHYGQKAILFLPLPVSSD

SEQ ID NO: 252 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLELLEENHYNTYISKKHAEKNWFVGLKKNGSCKRGPRTHYGQKAILFLPLPVSSD

SEQ ID NO: 253 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLELLEENHYVTYISKKHAEKNWFVGLKKNGSCKRGPRTHYGQKAILFLPLPVSSD

SEQ ID NO: 254 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLELLEENHYVTYISKKHAEKNWFVGLKKNGSVKRGPRTHYGQKAILFLPLPVSSD

SEQ ID NO: 255 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLELLEENHYNTYISKKHAEKNWFVGLKKNGSVKRGPRTHYGQKAILFLPLPVSSD

SEQ ID NO: 256 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLELLEENHYNTYISKKHAEKNWFVGLKKNGSVKRGPRTHYGQKAILFLPLPVSSD

SEQ ID NO: 257 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLELLEENHYNTYISKKHAEKNWFVGLKKNGSVKRGPRTHYGQKAILFLPLPVSSD

SEQ ID NO: 258 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLELLEENHVNTYISKKHAEKNWFVGLKKNGSVKRGPRTHYGQKAILFLPLPVSSD

SEQ ID NO: 259 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLEYLEENHYNTYISKKHAEKNWFVGLKKNGSCKRGPRTHYGQKAILFLPLPVSSD

SEQ ID NO: 260 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLEYLEENHYNTYISKKHAEKNWFVGLKKNGSCKRGPRTHYGQKAILFLPLPVSSD

SEQ ID NO: 261 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLEYLEENHYVTYISKKHAEKNWFVGLKKNGSCKRGPRTHYGQKAILFLPLPVSSD

SEQ ID NO: 262 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLEYLEENHYVTYISKKHAEKNWFVGLKKNGSVKRGPRTHYGQKAILFLPLPVSSD

SEQ ID NO: 263 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLEYLEENHYNTYISKKHAEKNWFVGLKKNGSVKRGPRTHYGQKAILFLPLPVSSD

SEQ ID NO: 264 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLEYLEENHYNTYISKKHAEKNWFVGLKKNGSVKRGPRTHYGQKAILFLPLPVSSD

SEQ ID NO: 265 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLEYLEENHYNTYISKKHAEKNWFVGLKKNGSVKRGPRTHYGQKAILFLPLPVSSD

SEQ ID NO: 266 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLEYLEENHVNTYISKKHAEKNWFVGLKKNGSVKRGPRTHYGQKAILFLPLPVSSD

SEQ ID NO: 267 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLEDLEENHYNTYISKKHAEKNWFVGLKKNGSCKRGPRTHYGQKAILFLPLPVSSD

SEQ ID NO: 268 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLEDLEENHYNTYISKKHAEKNWFVGLKKNGSCKRGPRTHYGQKAILFLPLPVSSD

SEQ ID NO: 269 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLEDLEENHYVTYISKKHAEKNWFVGLKKNGSCKRGPRTHYGQKAILFLPLPVSSD

SEQ ID NO: 270 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLEDLEENHYVTYISKKHAEKNWFVGLKKNGSVKRGPRTHYGQKAILFLPLPVSSD

SEQ ID NO: 271 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLEDLEENHYNTYISKKHAEKNWFVGLKKNGSVKRGPRTHYGQKAILFLPLPVSSD

SEQ ID NO: 272 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLEDLEENHYNTYISKKHAEKNWFVGLKKNGSVKRGPRTHYGQKAILFLPLPVSSD

SEQ ID NO: 273 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLEDLEENHYNTYISKKHAEKNWFVGLKKNGSVKRGPRTHYGQKAILFLPLPVSSD

SEQ ID NO: 274 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLEDLEENHVNTYISKKHAEKNWFVGLKKNGSVKRGPRTHYGQKAILFLPLPVSSD

SEQ ID NO: 275 - protein, 140 aa, Artificial Sequence
  Synthetic polypeptide
FNLPPGNYKKPKLLYCSNGGHFLRILPDGTVDGTRDRSDQHIKLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 276 - protein, 131 aa, Artificial Sequence
  Synthetic polypeptide
KPKLLYCSNGGHFLRILPDGTVDGTRDRSDQHIKLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 277 - protein, 140 aa, Artificial Sequence
  Synthetic polypeptide
FNLPPGNYKKPVLLYCSNGGHFLRILPDGTVDGTRDRSDQHIKLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 278 - protein, 131 aa, Artificial Sequence
  Synthetic polypeptide
KPVLLYCSNGGHFLRILPDGTVDGTRDRSDQHIKLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 279 - protein, 140 aa, Artificial Sequence
  Synthetic polypeptide
FNLPPGNYKKPKLLYCSNGGHFLRILPDGTVDGTRDRSDQHIKLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 280 - protein, 140 aa, Artificial Sequence
  Synthetic polypeptide
FNLPPGNYKKPKLLYCSNGGHFLRILPDGTVDGTRDRSDQHIKLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 281 - protein, 140 aa, Artificial Sequence
  Synthetic polypeptide
FNLPPGNYKKPKLLYCSNGGHFLRILPDGTVDGTEDRSDQHIKLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 282 - protein, 140 aa, Artificial Sequence
  Synthetic polypeptide
FNLPPGNYKKPVLLYCSNGGHFLRILPDGTVDGTRDRSDQHIKLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 283 - protein, 140 aa, Artificial Sequence
  Synthetic polypeptide
FNLPPGNYKKPKLLYCSNGGHFLRILPDGTVDGTRDRSDQHIALQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 284 - protein, 131 aa, Artificial Sequence
  Synthetic polypeptide
KPKLLYCSNGGHFLRILPDGTVDGTRDRSDQHIALQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 285 - protein, 140 aa, Artificial Sequence
  Synthetic polypeptide
FNLPPGNYKKPVLLYCSNGGHFLRILPDGTVDGTRDRSDQHIALQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 286 - protein, 131 aa, Artificial Sequence
  Synthetic polypeptide
KPVLLYCSNGGHFLRILPDGTVDGTRDRSDQHIALQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 287 - protein, 140 aa, Artificial Sequence
  Synthetic polypeptide
FNLPPGNYKKPKLLYCSNGGHFLRILPDGTVDGTRDRSDQHIALQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 288 - protein, 140 aa, Artificial Sequence
  Synthetic polypeptide
FNLPPGNYKKPKLLYCSNGGHFLRILPDGTVDGTRDRSDQHIALQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 289 - protein, 140 aa, Artificial Sequence
  Synthetic polypeptide
FNLPPGNYKKPKLLYCSNGGHFLRILPDGTVDGTEDRSDQHIALQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 290 - protein, 140 aa, Artificial Sequence
  Synthetic polypeptide
FNLPPGNYKKPVLLYCSNGGHFLRILPDGTVDGTRDRSDQHIALQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 291 - protein, 140 aa, Artificial Sequence
  Synthetic polypeptide
FNLPPGNYKKPKLLYCSNGGHFLRILPDGTVDGTRDRSDQHIELQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 292 - protein, 131 aa, Artificial Sequence
  Synthetic polypeptide
KPKLLYCSNGGHFLRILPDGTVDGTRDRSDQHIELQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 293 - protein, 140 aa, Artificial Sequence
  Synthetic polypeptide
FNLPPGNYKKPVLLYCSNGGHFLRILPDGTVDGTRDRSDQHIELQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 294 - protein, 131 aa, Artificial Sequence
  Synthetic polypeptide
KPVLLYCSNGGHFLRILPDGTVDGTRDRSDQHIELQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 295 - protein, 140 aa, Artificial Sequence
  Synthetic polypeptide
FNLPPGNYKKPKLLYCSNGGHFLRILPDGTVDGTRDRSDQHIELQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 296 - protein, 140 aa, Artificial Sequence
  Synthetic polypeptide
FNLPPGNYKKPKLLYCSNGGHFLRILPDGTVDGTRDRSDQHIELQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 297 - protein, 140 aa, Artificial Sequence
  Synthetic polypeptide
FNLPPGNYKKPKLLYCSNGGHFLRILPDGTVDGTEDRSDQHIELQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 298 - protein, 140 aa, Artificial Sequence
  Synthetic polypeptide
FNLPPGNYKKPVLLYCSNGGHFLRILPDGTVDGTRDRSDQHIELQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 299 - protein, 140 aa, Artificial Sequence
  Synthetic polypeptide
FNLPPGNYKKPKLLYCSNGGHFLRILPDGTVDGTRDRSDQHIQLQLAAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 300 - protein, 131 aa, Artificial Sequence
  Synthetic polypeptide
KPKLLYCSNGGHFLRILPDGTVDGTRDRSDQHIQLQLAAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 301 - protein, 140 aa, Artificial Sequence
  Synthetic polypeptide
FNLPPGNYKKPVLLYCSNGGHFLRILPDGTVDGTRDRSDQHIQLQLAAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 302 - protein, 131 aa, Artificial Sequence
  Synthetic polypeptide
KPVLLYCSNGGHFLRILPDGTVDGTRDRSDQHIQLQLAAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 303 - protein, 140 aa, Artificial Sequence
  Synthetic polypeptide
FNLPPGNYKKPKLLYCSNGGHFLRILPDGTVDGTRDRSDQHIQLQLAAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 304 - protein, 140 aa, Artificial Sequence
  Synthetic polypeptide
FNLPPGNYKKPKLLYCSNGGHFLRILPDGTVDGTRDRSDQHIQLQLAAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 305 - protein, 140 aa, Artificial Sequence
  Synthetic polypeptide
FNLPPGNYKKPKLLYCSNGGHFLRILPDGTVDGTEDRSDQHIQLQLAAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 306 - protein, 140 aa, Artificial Sequence
  Synthetic polypeptide
FNLPPGNYKKPVLLYCSNGGHFLRILPDGTVDGTRDRSDQHIQLQLAAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 307 - protein, 140 aa, Artificial Sequence
  Synthetic polypeptide
FNLPPGNYKKPKLLYCSNGGHFLRILPDGTVDGTRDRSDQHIQLQLVAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 308 - protein, 131 aa, Artificial Sequence
  Synthetic polypeptide
KPKLLYCSNGGHFLRILPDGTVDGTRDRSDQHIQLQLVAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 309 - protein, 140 aa, Artificial Sequence
  Synthetic polypeptide
FNLPPGNYKKPVLLYCSNGGHFLRILPDGTVDGTRDRSDQHIQLQLVAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 310 - protein, 131 aa, Artificial Sequence
  Synthetic polypeptide
KPVLLYCSNGGHFLRILPDGTVDGTRDRSDQHIQLQLVAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 311 - protein, 140 aa, Artificial Sequence
  Synthetic polypeptide
FNLPPGNYKKPKLLYCSNGGHFLRILPDGTVDGTRDRSDQHIQLQLVAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 312 - protein, 140 aa, Artificial Sequence
  Synthetic polypeptide
FNLPPGNYKKPKLLYCSNGGHFLRILPDGTVDGTRDRSDQHIQLQLVAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 313 - protein, 140 aa, Artificial Sequence
  Synthetic polypeptide
FNLPPGNYKKPKLLYCSNGGHFLRILPDGTVDGTEDRSDQHIQLQLVAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 314 - protein, 140 aa, Artificial Sequence
  Synthetic polypeptide
FNLPPGNYKKPVLLYCSNGGHFLRILPDGTVDGTRDRSDQHIQLQLVAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 315 - protein, 140 aa, Artificial Sequence
  Synthetic polypeptide
FNLPPGNYKKPKLLFCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 316 - protein, 131 aa, Artificial Sequence
  Synthetic polypeptide
KPKLLFCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 317 - protein, 140 aa, Artificial Sequence
  Synthetic polypeptide
FNLPPGNYKKPVLLFCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 318 - protein, 131 aa, Artificial Sequence
  Synthetic polypeptide
KPVLLFCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 319 - protein, 140 aa, Artificial Sequence
  Synthetic polypeptide
FNLPPGNYKKPKLLFCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 320 - protein, 140 aa, Artificial Sequence
  Synthetic polypeptide
FNLPPGNYKKPKLLFCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 321 - protein, 140 aa, Artificial Sequence
  Synthetic polypeptide
FNLPPGNYKKPKLLFCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 322 - protein, 140 aa, Artificial Sequence
  Synthetic polypeptide
FNLPPGNYKKPVLLFCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 323 - protein, 140 aa, Artificial Sequence
  Synthetic polypeptide
FNLPPGNYKKPKLLACSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 324 - protein, 131 aa, Artificial Sequence
  Synthetic polypeptide
KPKLLACSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 325 - protein, 140 aa, Artificial Sequence
  Synthetic polypeptide
FNLPPGNYKKPVLLACSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 326 - protein, 131 aa, Artificial Sequence
  Synthetic polypeptide
KPVLLACSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 327 - protein, 140 aa, Artificial Sequence
  Synthetic polypeptide
FNLPPGNYKKPKLLACSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 328 - protein, 140 aa, Artificial Sequence
  Synthetic polypeptide
FNLPPGNYKKPKLLACSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 329 - protein, 140 aa, Artificial Sequence
  Synthetic polypeptide
FNLPPGNYKKPKLLACSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 330 - protein, 140 aa, Artificial Sequence
  Synthetic polypeptide
FNLPPGNYKKPVLLACSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 331 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFVPLPVSSD

SEQ ID NO: 332 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFVPLPVSSD

SEQ ID NO: 333 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFVPLPVSSD

SEQ ID NO: 334 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFVPLPVSSD

SEQ ID NO: 335 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFVPLPVSSD

SEQ ID NO: 336 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFVPLPVSSD

SEQ ID NO: 337 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFVPLPVSSD

SEQ ID NO: 338 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFVPLPVSSD

SEQ ID NO: 339 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFAPLPVSSD

SEQ ID NO: 340 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFAPLPVSSD

SEQ ID NO: 341 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFAPLPVSSD

SEQ ID NO: 342 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFAPLPVSSD

SEQ ID NO: 343 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFAPLPVSSD

SEQ ID NO: 344 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFAPLPVSSD

SEQ ID NO: 345 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFAPLPVSSD

SEQ ID NO: 346 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFAPLPVSSD

SEQ ID NO: 347 - protein, 140 aa, Artificial Sequence
  Synthetic polypeptide
FNLPPGNYKKPKLLYCSNGGHFLRILPDGTVDGTKDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 348 - protein, 131 aa, Artificial Sequence
  Synthetic polypeptide
KPKLLYCSNGGHFLRILPDGTVDGTKDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 349 - protein, 140 aa, Artificial Sequence
  Synthetic polypeptide
FNLPPGNYKKPVLLYCSNGGHFLRILPDGTVDGTKDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 350 - protein, 131 aa, Artificial Sequence
  Synthetic polypeptide
KPVLLYCSNGGHFLRILPDGTVDGTKDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 351 - protein, 140 aa, Artificial Sequence
  Synthetic polypeptide
FNLPPGNYKKPKLLYCSNGGHFLRILPDGTVDGTKDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 352 - protein, 140 aa, Artificial Sequence
  Synthetic polypeptide
FNLPPGNYKKPKLLYCSNGGHFLRILPDGTVDGTKDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 353 - protein, 140 aa, Artificial Sequence; identical to SEQ ID NO: 351
  Synthetic polypeptide

SEQ ID NO: 354 - protein, 140 aa, Artificial Sequence
  Synthetic polypeptide
FNLPPGNYKKPVLLYCSNGGHFLRILPDGTVDGTKDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 355 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLQRLEENHYNTYISKKHAEKNWFVGLKKNGSCKRGPRTHYGQKAILFLPLPVSSD

SEQ ID NO: 356 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLQRLEENHYNTYISKKHAEKNWFVGLKKNGSCKRGPRTHYGQKAILFLPLPVSSD

SEQ ID NO: 357 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLQRLEENHYVTYISKKHAEKNWFVGLKKNGSCKRGPRTHYGQKAILFLPLPVSSD

SEQ ID NO: 358 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLQRLEENHYVTYISKKHAEKNWFVGLKKNGSVKRGPRTHYGQKAILFLPLPVSSD

SEQ ID NO: 359 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLQRLEENHYNTYISKKHAEKNWFVGLKKNGSVKRGPRTHYGQKAILFLPLPVSSD

SEQ ID NO: 360 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLQRLEENHYNTYISKKHAEKNWFVGLKKNGSVKRGPRTHYGQKAILFLPLPVSSD

SEQ ID NO: 361 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLQRLEENHYNTYISKKHAEKNWFVGLKKNGSVKRGPRTHYGQKAILFLPLPVSSD

SEQ ID NO: 362 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLQRLEENHVNTYISKKHAEKNWFVGLKKNGSVKRGPRTHYGQKAILFLPLPVSSD

SEQ ID NO: 363 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLDRLEENHYNTYISKKHAEKNWFVGLKKNGSCKRGPRTHYGQKAILFLPLPVSSD

SEQ ID NO: 364 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLDRLEENHYNTYISKKHAEKNWFVGLKKNGSCKRGPRTHYGQKAILFLPLPVSSD

SEQ ID NO: 365 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLDRLEENHYVTYISKKHAEKNWFVGLKKNGSCKRGPRTHYGQKAILFLPLPVSSD

SEQ ID NO: 366 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLDRLEENHYVTYISKKHAEKNWFVGLKKNGSVKRGPRTHYGQKAILFLPLPVSSD

SEQ ID NO: 367 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLDRLEENHYNTYISKKHAEKNWFVGLKKNGSVKRGPRTHYGQKAILFLPLPVSSD

SEQ ID NO: 368 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLCMDTDGLLYGSQTPNEECLFLDRLEENHYNTYISKKHAEKNWFVGLKKNGSVKRGPRTHYGQKAILFLPLPVSSD

SEQ ID NO: 369 - protein, 140 aa, Artificial Sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLDRLEENHYNTYISKKHAEKNWFVGLKKNGSVKRGPRTHYGQKAILFLPLPVSSD

SEQ ID NO: 370 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLDRLEENHVNTYISKKHAEKNWFVGLKKNGSVKRGPRTHYGQKAILFLPLPVSSD

SEQ ID NO: 371 - protein, 140 aa, Artificial Sequence
  Synthetic polypeptide
FNLPPGNFKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 372 - protein, 140 aa, Artificial Sequence
  Synthetic polypeptide
FNLPPGNFKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 373 - protein, 140 aa, Artificial Sequence
  Synthetic polypeptide
FNLPPGNFKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 374 - protein, 140 aa, Artificial Sequence
  Synthetic polypeptide
FNLPPGNFKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 375 - protein, 140 aa, Artificial Sequence
  Synthetic polypeptide
FNLPPGNFKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 376 - protein, 140 aa, Artificial Sequence
  Synthetic polypeptide
FNLPPGNFKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 377 - protein, 140 aa, Artificial Sequence
  Synthetic polypeptide
FNLPPGNVKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 378 - protein, 140 aa, Artificial Sequence
  Synthetic polypeptide
FNLPPGNVKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 379 - protein, 140 aa, Artificial Sequence
  Synthetic polypeptide
FNLPPGNVKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 380 - protein, 140 aa, Artificial Sequence
  Synthetic polypeptide
FNLPPGNVKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 381 - protein, 140 aa, Artificial Sequence
  Synthetic polypeptide
FNLPPGNVKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 382 - protein, 140 aa, Artificial Sequence
  Synthetic polypeptide
FNLPPGNVKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 383 - protein, 140 aa, Artificial Sequence
  Synthetic polypeptide
FNLPPGNAKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 384 - protein, 140 aa, Artificial Sequence
  Synthetic polypeptide
FNLPPGNAKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 385 - protein, 140 aa, Artificial Sequence
  Synthetic polypeptide
FNLPPGNAKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 386 - protein, 140 aa, Artificial Sequence
  Synthetic polypeptide
FNLPPGNAKKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 387 - protein, 140 aa, Artificial Sequence
  Synthetic polypeptide
FNLPPGNAKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 388 - protein, 140 aa, Artificial Sequence
  Synthetic polypeptide
FNLPPGNAKKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 389 - protein, 140 aa, Artificial Sequence
  Synthetic polypeptide
FNLPPGNYRKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 390 - protein, 140 aa, Artificial Sequence
  Synthetic polypeptide
FNLPPGNYRKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 391 - protein, 140 aa, Artificial Sequence
  Synthetic polypeptide
FNLPPGNYRKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 392 - protein, 140 aa, Artificial Sequence
  Synthetic polypeptide
FNLPPGNYRKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 393 - protein, 140 aa, Artificial Sequence
  Synthetic polypeptide
FNLPPGNYRKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 394 - protein, 140 aa, Artificial Sequence
  Synthetic polypeptide
FNLPPGNYRKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 395 - protein, 140 aa, Artificial Sequence
  Synthetic polypeptide
FNLPPGNYAKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 396 - protein, 140 aa, Artificial Sequence
  Synthetic polypeptide
FNLPPGNYAKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 397 - protein, 140 aa, Artificial Sequence
  Synthetic polypeptide
FNLPPGNYAKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 398 - protein, 140 aa, Artificial Sequence
  Synthetic polypeptide
FNLPPGNYAKPKLLYCSNGGHFLRILPDGTVDGTRDRSDQHIQLQLSAESVGEVYIKSTETGQYLCMDTDGLLYGSQTPNEECLFLERLEENHYNTYISKKHAEKNWFVGLKKNGSVKRGPRTHYGQKAILFLPLPVSSD

SEQ ID NO: 399 - protein, 140 aa, Artificial Sequence
  Synthetic polypeptide
FNLPPGNYAKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 400 - protein, 140 aa, Artificial Sequence
  Synthetic polypeptide
FNLPPGNYAKPVLLYCSNGGHFLRILPDGTVDGTRDRSDQHIQLQLSAESVGEVYIKSTETGQYLAMDTDGLLYGSQTPNEECLFLERLEENHVNTYISKKHAEKNWFVGLKKNGSVKRGPRTHYGQKAILFLPLPVSSD

SEQ ID NO: 401 - protein, 131 aa, Artificial Sequence
  Synthetic polypeptide
KPKLLYCSNGGHFLRILPDGTVDGTRDRSDQHIQLQLSAESVGEVYIKSTETGQYLCMDTDGLLYGSQTPNEECLFLERLEENHYVTYISKKHAEKNWFVGLKKNGRVKRGPRTHYGQKAILFLPLPVSSD

SEQ ID NO: 402 - protein, 131 aa, Artificial Sequence
  Synthetic polypeptide
KPVLLYCSNGGYFLRILPDGTVDGTRDRSDQHIQFQLSAESVGEVYIKSTETGQYLCMDTDGLLYGSQTPNEECLFLERLEENHYVTYISKKYAEKNWYVGLKKNGSVKRGPRTHYGQKAILFLPLPVSSD

SEQ ID NO: 403 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGRCKRGPRTHYGQKAILFLPLPVSSD

SEQ ID NO: 404 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 405 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 406 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 407 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYTTYISKKHAEKNWFVGLKKNGRVKRGPRTHYGQKAILFLPLPVSSD

SEQ ID NO: 408 - protein, 140 aa, Artificial Sequence
  Synthetic polypeptide
FNLPPGNYKKPKLLYCSNGGHFLRILPDGTVDGTRDRSDQHIQLQLSAESVGEVAIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 409 - protein, 140 aa, Artificial Sequence
  Synthetic polypeptide
FNLPPGNYKKPKLLYCSNGGHFLRILPDGTVDGTRDRSDQHIQLQLSAESVGEVWIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 410 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLHRLEENHYNTYISKKHAEKNWFVGLKKNGRVKRGPRTHYGQKAILFLPLPVSSD

SEQ ID NO: 411 - protein, 131 aa, Artificial Sequence
  Synthetic polypeptide
KPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 412 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 413 - protein, 140 aa, Artificial Sequence
  Synthetic polypeptide
FNLPPGNYKKPKLLYCSNGGHFLRILPDGTVDGTRDRSDQHIQLQLSAASVGEVYIKSTETGQYLAMDTDGLLYGSQTPNEECLFLERLEENHYNTYISKKHAEKNWFVGLKKNGRVKRGPRTHYGQKAILFLPLPVSSD

SEQ ID NO: 414 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHVVTYISKKHAEKNWFVGLKKNGRVKRGPRTHYGQKAILFLPLPVSSD

SEQ ID NO: 415 - protein, 131 aa, Artificial Sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGRVKRGPRTHYGQKAILFLPLPVSSD

SEQ ID NO: 416 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 417 - protein, 140 aa, Artificial Sequence
  Synthetic polypeptide
FNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 418 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 419 - protein, 140 aa, Artificial Sequence; identical to SEQ ID NO: 30
  Synthetic polypeptide

SEQ ID NO: 420 - protein, 140 aa, Artificial Sequence
  Synthetic polypeptide
SYNHLQGDVRPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 421 - protein, 140 aa, Artificial Sequence
  Synthetic polypeptide
SYNHLQGDVRVVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 422 - protein, 141 aa, Artificial Sequence
  Synthetic polypeptide
SYDYMEGGDIRVVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 423 - protein, 39 aa, Artificial Sequence
  Synthetic polypeptide
HGEGTFTSDLSKQMEEEAVRLFIEWLKNGGPSSGAPPPS

SEQ ID NO: 424 - protein, 185 aa, Artificial Sequence
  Synthetic polypeptide
HGEGTFTSDLSKQMEEEAVRLFIEWLKNGGPSSGAPPPSGSGSGSFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 425 - protein, 185 aa, Artificial Sequence; identical to SEQ ID NO: 424
  Synthetic polypeptide

SEQ ID NO: 426 - protein, 177 aa, Artificial Sequence
  Synthetic polypeptide
DLSKQMEEEAVRLFIEWLKNGGPSSGAPPPSGSGSGSFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 427 - protein, 185 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSDGSGSGSHGEGTFTSDLSKQMEEEAVRLFIEWLKNGGPSSGAPPPS

SEQ ID NO: 428 - protein, 185 aa, Artificial Sequence
  Synthetic polypeptide
HGEGTFTSDLSKQMEEEAVRLFIEWLKNGGPSSGAPPPSGSGSGSFNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 429 - protein, 185 aa, Artificial Sequence
  Synthetic polypeptide
HGEGTFTSDLSKQMEEEAVRLFIEWLKNGGPSSGAPPPSGSGSGSFNLPPGNYKKPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 430 - protein, 182 aa, Artificial Sequence
  Synthetic polypeptide
HGEGTFTSDLSKQMEEEAVRLFIEWLKNGGPSSGAPPPSGSGSGSMRDSSPLPVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 431 - protein, 185 aa, Artificial Sequence
  Synthetic polypeptide
HGEGTFTSDLSKQMEEEAVRLFIEWLKNGGPSSGAPPPSGSGSGSSYNHLQGDVRVVLLYCSNGGYFLRILPDGTVDGTRDRSDQHIQFQLSAESVGEVYIKSTETGQYLAMDTDGLLYGSQTPNEECLFLERLEENHYVTYISKKYAEKNWYVGLKKNGSVKRGPRTHYGQKAILFLPLPVSSD

SEQ ID NO: 432 - protein, 183 aa, Artificial Sequence
  Synthetic polypeptide
HSEGTFTSDYSKYLDSRRAQDFVQWLMNTKRNRNNIAGSGSGSFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 433 - protein, 180 aa, Artificial Sequence
  Synthetic polypeptide
IKPEAPGEDASPEELNRYYASLRHYLNLVTRQRYGSGSGSFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLPLPVSSD

SEQ ID NO: 434 - protein, 31 aa, Artificial Sequence
  Synthetic polypeptide
DLSKQMEEEAVRLFIEWLKNGGPSSGAPPPS

SEQ ID NO: 435 - protein, 37 aa, Artificial Sequence
  Synthetic polypeptide
HSEGTFTSDYSKYLDSRRAQDFVQWLMNTKRNRNNIA

SEQ ID NO: 436 - protein, 34 aa, Artificial Sequence
  Synthetic polypeptide
IKPEAPGEDASPEELNRYYASLRHYLNLVTRQRY

SEQ ID NO: 437 - protein, 31 aa, Artificial Sequence
  Synthetic polypeptide
HAEGTFTSDVSSYLEGQAAKEFIAWLVKGRG

SEQ ID NO: 438 - protein, 30 aa, Artificial Sequence
  Synthetic polypeptide
HAEGTFTSDVSSYLEGQAAKEFIAWLVKGR

SEQ ID NO: 439 - protein, 6 aa, Artificial Sequence
  Synthetic peptide
GSGSGS

SEQ ID NO: 440 - protein, 10 aa, Artificial Sequence
  Synthetic peptide
GGSGGGGSGG

(19) United States
(12) Patent Application Publication
(10) Pub. No.: US 2019/0276510 A1
(43) Pub. Date: Sep. 12, 2019

(54) USE OF FIBROBLAST GROWTH FACTOR 1 (FGF1)-VAGUS NERVE TARGETING CHIMERIC PROTEINS TO TREAT HYPERGLYCEMIA

(71) Applicant: Salk Institute for Biological Studies, La Jolla, CA (US)

(72) Inventors: Ronald M. Evans, La Jolla, CA (US); Michael Downes, San Diego, CA (US); Annette Atkins, San Diego, CA (US); Ruth T. Yu, La Jolla, CA (US); Sihao Liu, San Diego, CA (US)

(73) Assignee: Salk Institute for Biological Studies, La Jolla, CA (US)

(21) Appl. No.: 16/419,929

(22) Filed: May 22, 2019

Related U.S. Application Data
(63) Continuation of application No. PCT/US2017/066417, filed on Dec. 14, 2017.
(60) Provisional application No. 62/434,512, filed on Dec. 15, 2016.

(51) Int. Cl. C07K 14/50; A61K 38/26; A61K 47/64; A61P 3/10; A61P 3/04

(52) U.S. Cl. CPC C07K 14/501; A61K 38/26; A61P 3/04; A61P 3/10; A61K 47/642

(57) ABSTRACT

The present disclosure provides FGF1 mutant proteins, which include an N-terminal deletion, point mutation(s), or combinations thereof, as well as FGF1-vagus targeting chimeric proteins which include an FGF1 portion (e.g., native FGF1 or mutant FGF1) and a portion that targets the chimera to the vagus nerve (e.g., GLP or exendin-4). Also provided are nucleic acid molecules that encode such proteins, and vectors and cells that include such nucleic acids. The disclosed FGF1 mutants and FGF1-vagus targeting chimeric proteins can reduce blood glucose in a mammal, and in some examples are used to treat a metabolic disorder.

### CROSS-REFERENCE TO RELATED APPLICATION

This application is a continuation of International Application No. PCT/US2017/066417, filed Dec. 14, 2017, which claims priority to U.S. Provisional Application No. 62/434,512 filed Dec. 15, 2016, both herein incorporated by reference.

### FIELD

This application provides FGF1 mutant proteins and FGF1-vagus targeting chimeric proteins, nucleic acids encoding such proteins, and their use for reducing blood glucose and/or treating a metabolic disease, for example in a diabetic patient.

### BACKGROUND

Type 2 diabetes and obesity are leading causes of mortality and are associated with the Western lifestyle, which is characterized by excessive nutritional intake and lack of exercise. A central player in the pathophysiology of these diseases is the nuclear hormone receptor (NHR) PPARγ, a lipid sensor and master regulator of adipogenesis. PPARγ is also the molecular target for the thiazolidinedione (TZD)-class of insulin sensitizers, which command a large share of the current oral anti-diabetic drug market. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia. Thus, the identification of new insulin sensitizers is needed.

Glucagon-like peptide 1 (GLP-1) is secreted postprandially from intestinal L cells to stimulate the secretion of insulin from pancreatic β cells. In addition, GLP-1 improves the function of β cells. In vivo, GLP-1 is rapidly degraded by DPP-IV, limiting its half-life to minutes. Analogs of GLP-1, including those resistant to DPP-IV degradation such as exendin-4, are currently used to treat hyperglycemia in type 2 diabetic patients.

### SUMMARY

It was previously observed that administration of FGF1, as well as FGF1 mutant proteins, lowers blood glucose levels in diabetic mammals in an insulin-dependent manner. It is shown herein that fusions of GLP-1 analogs (e.g., peptides that target the vagus nerve) with FGF1 analogs provide superior glucose control in diabetic mammals. These chimeric proteins are referred to herein as FGF1-vagus targeting chimeric proteins. FGF1-vagus targeting chimeric proteins are effective glucose lowering agents for the treatment of diabetes. Therapeutic dosing with GLP-1 analogs is normally twice per day, while FGF1 rapidly lowers glucose for several days. Fusing the vagus—targeting peptide exendin-4 via a flexible linker to FGF1 resulted in a protein able to reduce blood glucose levels for up to two weeks from a single injection. In addition, the FGF1-vagus targeting chimeric protein did not induce hypoglycemia, offering a safety advantage over existing diabetic treatments.

Based on these observations, mutant FGF1 proteins and FGF1-vagus targeting chimeric proteins (as well as nucleic acid molecules encoding such) are provided. Mutant FGF1 proteins can include an N-terminal truncation, one or more point mutation(s) (such as those in Table 1), or combinations thereof. In some examples, the FGF1 mutants are mutated to reduce the mitogenic activity, alter heparan sulfate and/or heparin binding, and/or increase the thermostability of the FGF1 mutant protein (e.g., relative to a native FGF1 protein). Specific FGF1 mutant proteins are provided in SEQ ID NOS: 10-422, such as SEQ ID NO: 420, 421 and 422. FGF1-vagus targeting chimeric proteins include an FGF1 portion (such as a native FGF1 protein or a mutant

FGF1 protein provided herein), and a portion that targets the chimera to the vagus nerve. In some examples, the chimera binds to the vagus nerve. FGF1-vagus targeting chimeric proteins can be generated for example, using a vagus nerve targeting peptide from the first column of Table 2 linked or attached to an FGF protein from the second column of Table 2. Specific examples of FGF1-vagus targeting chimeric proteins are provided in SEQ ID NOS: 424, 425, 426, 427, 428, 429, 430, 431, 432, and 433.

Methods of using the mutant FGF1 proteins and the FGF1-vagus targeting chimeric proteins, or nucleic acid molecules encoding such, for reducing blood glucose in a mammal, for example to treat a metabolic disease, are disclosed. Such FGF1 mutants and FGF1-vagus targeting chimeric proteins can be used alone, in combination, or in combination with other agents, such as other glucose reducing agents, such as thiazolidinedione. In some examples, use of the disclosed mutant FGF1 proteins or FGF1-vagus targeting chimeric proteins result in one or more of: reduction in triglycerides, decrease in insulin resistance, reduction of hyperinsulinemia, increase in glucose tolerance, reduction of food intake, or reduction of hyperglycemia in a mammal. In some examples, 1, 2, 3, 4 or 5 different FGF1-vagus targeting chimeric proteins are used. In some examples, 1, 2, 3, 4 or 5 different FGF1 mutant proteins are used.

Provided herein are mutated FGF1 proteins containing an N-terminal truncation, one or more point mutation(s) (such as amino acid substitutions, deletions, additions, or combinations thereof), or combinations of N-terminal deletions and point mutation(s). In some examples, such mutated FGF1 proteins have reduced mitogenicity relative to mature FGF1 (e.g., SEQ ID NO: 5), such as a reduction of at least 20%, at least 50%, at least 75% or at least 90%. In some examples, mutated FGF1 proteins have increased thermostability relative to mature FGF1 (e.g., SEQ ID NO: 5), such as an increase of at least 20%, at least 50%, at least 75%, at least 90%, at least 100%, or at least 200%. In some examples, the mutant FGF1 protein can include for example deletion of at least 5, at least 6, at least 10, at least 11, at least 12, at least 13, at least 14, at least 15, at least 16, at least 17, at least 18, at least 19, or at least 20 consecutive N-terminal amino acids. In some examples, the mutant FGF1 protein includes point mutations, such as one containing at least 1, at least 2, at least 3, at least 4, at least 5, at least 6, at least 7, at least 8, at least 9, or at least 10 additional amino acid substitutions (such as 1, 2, 3, 4, 5, 6, 7, 8, 9, 10, 11, 12, 13, 14, 15, 16, 17, 18, or 19 substitutions), such as one or more of those shown in Table 1. In some examples, the mutant FGF1 protein includes both an N-terminal truncation and one or more additional point mutations. In some examples, the mutant FGF1 protein includes at least 90, at least 100, or at least 110 consecutive amino acids from amino acids 5-141 of FGF1 (e.g., of SEQ ID NO: 2, 4 or 5), (which in some examples can include 1-20 point mutations, such as substitutions, deletions, and/or additions). In some examples, the mutated FGF1 protein has at least 80%, at least 85%, at least 90%, at least 95%, at least 96%, at least 97%, at least 99%, or 100% sequence identity to any one of SEQ ID NOS: 420, 421 or 422.

FGF1-vagus-targeting chimeric proteins are also provided herein. Such proteins include an FGF1 portion, and a portion that allows the chimera to target the vagus nerve. The two portions can be joined directly, or indirectly, for example via a spacer/linker. In some examples, the FGF1 portion is a native mature FGF1 protein (e.g., SEQ ID NO: 5). In some examples, the FGF1 portion is a mutated mature FGF1 protein, such as one having at least 80%, at least 85%, at least 90%, at least 95%, at least 96%, at least 97%, at least 99%, or 100% sequence identity to any one of SEQ ID NOS: 10-422 (and in some examples where the variant retains the point mutation(s) recited herein for that sequence). In some examples, the vagus nerve targeting portion has at least 80%, at least 85%, at least 90%, at least 95%, at least 96%, at least 97%, at least 99%, or 100% sequence identity to SEQ ID NO: 423, 434, 435, 436, 437, or 438 wherein the variant retains the ability to target the chimeric protein to the vagus nerve. In some examples, the FGF1-vagus targeting chimeric protein has at least 80%, at least 85%, at least 90%, at least 95%, at least 96%, at least 97%, at least 99%, or 100% sequence identity to any one of SEQ ID NOS: 424, 425, 426, 427, 428, 429, 430, 431, 432, and 433.

Also provided are isolated nucleic acid molecules encoding the disclosed mutant FGF1 proteins and FGF1-vagus targeting chimeric proteins. Vectors and cells that include such nucleic acid molecules are also provided.

Methods of using the disclosed mutant FGF1 proteins and FGF1-vagus targeting chimeric proteins (or nucleic acid molecules encoding such) are provided. In some examples the methods include administering a therapeutically effective amount of one or more disclosed mutant FGF1 proteins and/or FGF1-vagus targeting chimeric proteins (or nucleic acid molecules encoding such) to reduce blood glucose in a mammal, such as a decrease of at least 5%, at least 10%, at least 25%, at least 50%, or at least 75%. In some examples, the glucose lowering effect lasts at least 5 days, at least 7 days, at least 14 days, at least 21 days, or even at least 30 days. In some examples the methods include administering a therapeutically effective amount of a disclosed mutant FGF1 protein and/or FGF1-vagus targeting chimeric protein (or nucleic acid molecules encoding such) to treat a metabolic disease in a mammal. Exemplary metabolic diseases that can be treated with the disclosed methods include, but are not limited to: diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension). In some examples, one or more of these diseases are treated simultaneously with the disclosed FGF1 mutant proteins and/or FGF1-vagus targeting chimeric proteins. Also provided are methods of reducing fed and fasting blood glucose, improving insulin sensitivity and glucose tolerance, reducing systemic chronic inflammation, ameliorating hepatic steatosis in a mammal, reducing food intake, or combinations thereof, by administering a therapeutically effective amount of a disclosed mutant FGF1 protein and/or FGF1-vagus targeting chimeric protein (or nucleic acid molecules encoding such).

The foregoing and other objects and features of the disclosure will become more apparent from the following detailed description, which proceeds with reference to the accompanying figures.

### BRIEF DESCRIPTION OF THE DRAWINGS

FIG. 1 shows an alignment between different mammalian wild-type FGF1 sequences (human (SEQ ID NO: 2), gorilla (SEQ ID NO: 6), chimpanzee (SEQ ID NO: 7), canine (SEQ ID NO: 8), feline (SEQ ID NO: 8), and mouse (SEQ ID NO: 4)). Similar alignments can be generated and used to make the mutations provided herein to any FGF1 sequence of interest.

FIGS. 2A-2C show exemplary FGF1 mutant proteins that have the N-terminal sequence replaced with a peptide designed to target the b splice variant of FGFR1, and point mutations K12V, H21Y, L44F, N95V, H102Y, F108Y, and C117V. (A) SEQ ID NO: 420, (B) SEQ ID NO: 421, and (C) SEQ ID NO: 422.

FIG. 3A shows the protein sequence of a dipeptidyl peptidase IV (DPP4) resistant GLP1 analog exendin-4 (SEQ ID NO: 423), derived from the glia monster, that has prolonged in vivo efficacy compared to GLP-1.

FIG. 3B shows an FGF1-vagus targeting chimeric protein sequence (SEQ ID NO: 424) comprising an N-terminal vagus nerve targeting sequence (SEQ ID NO: 423), a linker (underlined), and a mature FGF1 sequence (SEQ ID NO: 5).

FIGS. 4A-4H show exemplary FGF1-vagus chimeric proteins that include a vagus nerve targeting sequence, a six amino acid flexible linker (GSGSGS), and a mutant FGF1 sequence. (A) The vagus nerve targeting sequence is an N-terminally truncated version of exendin 4 (amino acids 9-39) that restricts receptor internalization, and the mutant FGF1 is a mature FGF1 sequence with a C117V mutation (SEQ ID NO: 426). (B) FGF1-vagus targeting chimeric protein sequence comprising a C-terminal vagus nerve targeting sequence (SEQ ID NO: 423), a linker (underlined), and a mutant FGF mature sequence with a C117V mutation (SEQ ID NO: 427). (C) The vagus nerve targeting sequence is exendin 4, and the mutant FGF1 is a mature FGF1 sequence with mutations K12V, N95V, and C117V (SEQ ID NO: 428). (D) The vagus nerve targeting sequence is exendin 4, and the mutant FGF1 is a mature FGF1 sequence with mutations K12V, H21Y, L44F, N95V, H102Y, F108Y, and C117V (SEQ ID NO: 429). (E) The vagus nerve targeting sequence is exendin 4, and the mutant FGF1 is an N-terminally truncated FGF1 sequence (with the deleted amino acids replaced with MRDSSPL) with mutations K12V, H21Y, L44F, N95V, H102Y, F108Y, and C117V (SEQ ID NO: 430). (F) The vagus nerve targeting sequence is exendin 4, and the mutant FGF1 is an N-terminally truncated FGF1 sequence (with the deleted amino acids replaced with SYNHLQGDVRV, an FGF10 sequence that targets FGFR1b) with mutations K12V, H21Y, L44F, N95V, H102Y, F108Y, and C117V (SEQ ID NO: 431). (G) The vagus nerve targeting sequence is oxyntomodulin (SEQ ID NO: 435), and the mutant FGF1 is a mature FGF1 with mutation C117V (SEQ ID NO: 432). (H) The vagus nerve targeting sequence is PYY (SEQ ID NO: 436), and the mutant FGF is a mature FGF1 with mutation C117V (SEQ ID NO: 433).

FIGS. 5A-5D are graphs showing the in vivo blood glucose lowering effects of human FGF1 (SEQ ID NO: 5) and GLP1-FGF1 chimera (SEQ ID NO: 424). (A) % of initial blood glucose over 4 hours, (B) % of initial blood glucose over 35 days, or (C) % of initial blood glucose over 5 days. (D) Dose response of GLP1-FGF1 chimera (SEQ ID NO: 424) on blood glucose levels at baseline and 34 days following administration.

FIGS. 6A-6L are graphs showing the effect of FGF1 (SEQ ID NO: 5; 0.5 mg/kg) and a GLP1-FGF1 chimera (SEQ ID NO: 424; 0.1, 0.25, 0.63, or 1 mg/kg) on blood glucose acutely (over 48 hours) or chronically (up to 400 hours).

FIGS. 7A-7D are graphs showing the effect of FGF1 (SEQ ID NO: 5; 0.5 mg/kg) and a GLP1-FGF1 chimera (SEQ ID NO: 424; 0.1, 0.25, 0.63, or 1 mg/kg) on (A) insulin levels and (B-D) body weight.

FIGS. 8A-8F are graphs showing the effect of FGF1 (SEQ ID NO: 5; 0.5 mg/kg) and a GLP1-FGF1 chimera (SEQ ID NO: 424; 0.1, 0.25, 0.63, or 1 mg/kg) on glucose tolerance measured 15 days post injection and following 10 hours of fasting.

FIG. 8G is a bar graph showing the glucose tolerance, as measured by the average area under the curve (AUC) for FGF1 (SEQ ID NO: 5; 0.5 mg/kg) and a GLP1-FGF1 chimera (SEQ ID NO: 424; 0.1, 0.25, 0.63, or 1 mg/kg) measured 15 days post injection and following 10 hours of fasting.

FIGS. 9A-9F are graphs showing the effect of FGF1 (SEQ ID NO: 5; 0.5 mg/kg) and a GLP1-FGF1 chimera (SEQ ID NO: 424; 0.1, 0.25, 0.63, or 1 mg/kg) on pyruvate tolerance test (PTT) measured 20 days post injection and 16 hours of fasting.

FIG. 9G is a bar graph showing PTT AUC averages for FGF1 (SEQ ID NO: 5; 0.5 mg/kg) and a GLP1-FGF1 chimera (SEQ ID NO: 424; 0.1, 0.25, 0.63, or 1 mg/kg) measured 20 days post injection and 16 hours of fasting.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing filed herewith (generated on May 8, 2019, 551 KB), is incorporated by reference in its entirety.

SEQ ID NOS: 1 and 2 provide an exemplary human FGF1 nucleic acid and protein sequences, respectively. Source: GenBank Accession Nos: BC032697.1 and AAH32697.1. Heparan binding residues are amino acids 127-129 and 133-134.

SEQ ID NOS: 3 and 4 provide an exemplary mouse FGF1 nucleic acid and protein sequences, respectively. Source: GenBank Accession Nos: BC037601.1 and AAH37601.1.

SEQ ID NO: 5 provides an exemplary mature form of human FGF1 (140 aa, sometimes referred to in the art as FGF1 15-154)

SEQ ID NO: 6 provides an exemplary gorilla FGF1 protein sequence.

SEQ ID NO: 7 provides an exemplary chimpanzee FGF1 protein sequence.

SEQ ID NO: 8 provides an exemplary dog FGF1 protein sequence.

SEQ ID NO: 9 provides an exemplary cat FGF1 protein sequence.

SEQ ID NO: 10 (Salk_075) provides an exemplary mature form of FGF1 with point mutations (K12V, A66C, N95V, C117V) wherein numbering refers to SEQ ID NO: 5.

SEQ ID NO: 11 (Salk_076) provides an exemplary mature form of FGF1 with point mutations Y55W, E87H, S116R, and C117V, wherein numbering refers to SEQ ID NO: 5.

SEQ ID NO: 12 (Salk_077) provides an exemplary mature form of FGF1 with point mutations K12V, Y55W, N95V, S116R, and C117V, wherein numbering refers to SEQ ID NO: 5.

SEQ ID NO: 13 (Salk_079) provides an exemplary N-terminally truncated form of FGF1 with point mutations K12V, L44F, C83T, N95V, C117V, and F132W, wherein numbering refers to SEQ ID NO: 5, wherein some of the N-terminus is replaced with an engineered N-terminal sequence (MRDSSPL, referred to as NF21).

SEQ ID NO: 14 (Salk_080) provides an exemplary N-terminally truncated form of FGF1 with point mutations K12V, H21Y, L44F, N95V, H102Y, F108Y, and C117V, wherein numbering refers to SEQ ID NO: 5, wherein some of the N-terminus is replaced with NF21.

SEQ ID NO: 15 (Salk_081) provides an exemplary N-terminally truncated form of FGF1 with point mutations K12V, E87V, and C117V, wherein numbering refers to SEQ ID NO: 5, wherein some of the N-terminus is replaced with NF21.

SEQ ID NO: 16 (Salk_102_1) provides an exemplary N-terminally truncated form of FGF1 with point mutations Q40P, S47I, H93G, and N95V, wherein numbering refers to SEQ ID NO: 5.

SEQ ID NO: 17 (Salk_102_2) provides an exemplary N-terminally truncated form of FGF1 with point mutations (H21Y, L44F, N95V, H102Y, and F108Y, wherein numbering refers to SEQ ID NO: 5.

SEQ ID NO: 18 (Salk_102_3) provides an exemplary N-terminally truncated form of FGF1 with point mutations (H21Y, L44F, N95V, H102Y, F108Y, and C117V, wherein numbering refers to SEQ ID NO: 5).

SEQ ID NO: 19 (Salk_102_4) provides an exemplary N-terminally truncated form of FGF1 with point mutations L44F, C83T, N95V, F132W, and C117V, wherein numbering refers to SEQ ID NO: 5.

SEQ ID NO: 20 (Salk_102_5) provides an exemplary N-terminally truncated form of FGF1 with point mutations H21Y, L44F, N95V, H102Y, F108Y, and C117V wherein numbering refers to SEQ ID NO: 5, wherein some of the N-terminus is replaced with NF21.

SEQ ID NO: 21 (Salk_102_6) provides an exemplary N-terminally truncated form of FGF1 with point mutations H21Y, L44F, N95V, H102Y, and F108Y, wherein numbering refers to SEQ ID NO: 5, wherein some of the N-terminus is replaced with NF21.

SEQ ID NO: 22 (Salk_103_1) provides an exemplary N-terminally truncated form of FGF1 with point mutations K12V, Q40P, S47I, H93G, and N95V, wherein numbering refers to SEQ ID NO: 5.

SEQ ID NO: 23 (Salk_103_2) provides an exemplary N-terminally truncated form of FGF1 with point mutations K12V, H21Y, L44F, N95V, H102Y, and F108Y, wherein numbering refers to SEQ ID NO: 5.

SEQ ID NO: 24 (Salk_103_3) provides an exemplary N-terminally truncated form of FGF1 with point mutations K12 and N95V, wherein numbering refers to SEQ ID NO: 5.

SEQ ID NO: 25 provides an exemplary mature form of FGF1 with point mutations S99A, K101E, H102A, and W107A, wherein numbering refers to SEQ ID NO: 5. One, two, three of all four of these point mutations can be made to an FGF1 sequence (such as a mutant FGF1 protein provided herein) for example to reduce its mitogenicity.

SEQ ID NO: 26 provides an exemplary mature form of FGF1 with an N-terminal deletion.

SEQ ID NO: 27 provides an exemplary mature form of FGF1 with an N-terminal deletion (FGF1^{ΔNT}(10-140αα)).

SEQ ID NO: 28 provides an exemplary mature form of FGF1 with an N-terminal deletion (FGF1^{ΔNT2}(14-140αα)).

SEQ ID NO: 29 provides an exemplary mature form of FGF1 with an N-terminal deletion (FGF1^{ΔNT3}(12-140αα)).

SEQ ID NO: 30 provides an exemplary mature form of FGF1 with point mutations K12V and N95V (wherein numbering refers to SEQ ID NO: 5) for example to reduce mitogenic activity.

SEQ ID NO: 31 provides an exemplary mature form of FGF1 with point mutations K12V, L46V, E87V, N95V, P134V (wherein numbering refers to SEQ ID NO: 5) for example to reduce mitogenic activity.

SEQ ID NOS: 32 and 33 provide exemplary mature forms of FGF1 with mutations in the heparan binding domain (K118N or K118E, respectively, wherein numbering refers to SEQ ID NO: 5). In some examples these sequences further include MFNLPPG at their N-terminus. Such proteins can have reduced mitogenicity as compared to wild-type FGF1.

SEQ ID NO: 34 provides an exemplary N-terminally truncated form of FGF1, wherein the four N-terminal amino acids are from FGF21.

SEQ ID NO: 35 provides an exemplary mature form of FGF1 with point mutations K12V, C117V and P134V (wherein numbering refers to SEQ ID NO: 5) for example to reduce mitogenic activity and increase thermostability. From Xia et al., PLoS One. 7(11):e48210, 2012.

SEQ ID NO: 36 (FGF1(1-140αα)M1a) provides an exemplary mature form of FGF1 with point mutations K12V, N95V, C117V, and P134V (wherein numbering refers to SEQ ID NO: 5) for example to reduce mitogenic activity and increase thermostability.

SEQ ID NO: 37 (FGF1^{ΔNT1} (1-140αα)M1) provides an exemplary N-terminally truncated form of FGF1 with point mutations (K12V, C117V, and P134V wherein numbering refers to SEQ ID NO: 5) for example for example to reduce mitogenic activity and increase thermostability.

SEQ ID NO: 38 (FGF1^{ΔNT3} (1-140αα)M1a) provides an exemplary N-terminally truncated form of FGF1 with point mutations (K12V, C117V, and P134V wherein numbering refers to SEQ ID NO: 5) for example to reduce mitogenic activity and increase thermostability.

SEQ ID NO: 39 (FGF1^{ΔNT1} (1-140αα)M1a) provides an exemplary N-terminally truncated form of FGF1 with point mutations (K12V, N95V, C117V, and P134V wherein numbering refers to SEQ ID NO: 5) for example to reduce mitogenic activity, and increase thermostability.

SEQ ID NO: 40 (FGF1^{ΔNT3} (1-140αα)M1a) provides an exemplary N-terminally truncated form of FGF1 with point mutations K12V, N95V, C117V, and P134V (wherein numbering refers to SEQ ID NO: 5) for example to reduce mitogenic activity, and increase thermostability.

SEQ ID NO: 41 (FGF1(1-140αα)M2) provides an exemplary mature form of FGF1 with point mutations L44F, C83T, C117V, and F132W (wherein numbering refers to SEQ ID NO: 5) for example to reduce mitogenic activity and increase thermostability. From Xia et al., PLoS One. 7(11):e48210, 2012.

SEQ ID NO: 42 (FGF1(1-140αα)M2a) provides an exemplary mature form of FGF1 with point mutations (L44F, C83T, N95V, C117V, and F132W wherein numbering refers to SEQ ID NO: 5) for example to reduce mitogenic activity and increase thermostability.

SEQ ID NO: 43 (FGF1(1-140αα)M2b) provides an exemplary mature form of FGF1 with point mutations K12V, L44F, C83T, C117V, and F132W, wherein numbering refers to SEQ ID NO: 5, for example to reduce mitogenic activity and increase thermostability.

SEQ ID NO: 44 (FGF1(1-140αα)M2c) provides an exemplary mature form of FGF1 with point mutations (K12V, L44F, C83T, N95V, C117V, and F132W wherein numbering refers to SEQ ID NO: 5) for example to reduce mitogenic activity and increase thermostability.

SEQ ID NO: 45 (FGF1^{ΔNT1}(10-140αα)M2) provides an exemplary N-terminally truncated form of FGF1 with point mutations (L44F, C83T, C117V, and F132W wherein numbering refers to SEQ ID NO: 5) for example to reduce mitogenic activity and increase thermostability.

SEQ ID NO: 46 (FGF1^{ΔNT3}(12-140αα)M2) provides an exemplary N-terminally truncated form of FGF1 with point mutations (L44F, C83T, C117V, and F132W wherein numbering refers to SEQ ID NO: 5) for example to reduce mitogenic activity and increase thermostability.

SEQ ID NO: 47 (FGF1^{ΔNT1}(10-140αα)M2a) provides an exemplary N-terminally truncated form of FGF1 with point mutations (L44F, C83T, N95V, C117V, and F132W wherein numbering refers to SEQ ID NO: 5) for example to reduce mitogenic activity and increase thermostability.

SEQ ID NO: 48 (FGF1^{ΔNT3}(12-140αα)M2a) provides an exemplary N-terminally truncated form of FGF1 with point mutations (L44F, C83T, N95V, C117V, and F132W wherein numbering refers to SEQ ID NO: 5) for example to reduce mitogenic activity and increase thermostability.

SEQ ID NO: 49 (FGF1^{ΔNT1}(10-140αα)M2b) provides an exemplary N-terminally truncated form of FGF1 with point mutations (K12V, L44F, C83T, C117V, and F132W wherein numbering refers to SEQ ID NO: 5) for example to reduce mitogenic activity and increase thermostability.

SEQ ID NO: 50 (FGF1^{ΔNT3}(12-140αα)M2b) provides an exemplary N-terminally truncated form of FGF1 with point mutations (K12V, L44F, C83T, C117V, and F132W wherein numbering refers to SEQ ID NO: 5) for example to reduce mitogenic activity and increase thermostability.

SEQ ID NO: 51 (FGF1^{ΔNT1}(10-140αα)M2c) provides an exemplary N-terminally truncated form of FGF1 with point mutations (K12V, L44F, C83T, N95V, and C117V, F132W wherein numbering refers to SEQ ID NO: 5) for example to reduce mitogenic activity and increase thermostability.

SEQ ID NO: 52 (FGF1^{ΔNT3}(12-140αα)M2c) provides an exemplary N-terminally truncated form of FGF1 with point mutations (K12V, L44F, C83T, N95V, and C117V, F132W wherein numbering refers to SEQ ID NO: 5) for example to reduce mitogenic activity and increase thermostability.

SEQ ID NO: 53 (FGF1(1-140αα)M3) provides an exemplary mature form of FGF1 with mutations (L44F, M67I, L73V, V109L, L111I, C117V, A103G, R119G A104-106, and Δ120-122, wherein numbering refers to SEQ ID NO: 5) for example to reduce mitogenic activity and increase thermostability. From Xia et al., PLoS One. 7(11):e48210, 2012.

SEQ ID NO: 54 (FGF1(1-140αα)M3a) provides an exemplary mature form of FGF1 with mutations (K12V, L44F, M67I, L73V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 55 (FGF1(1-140αα)M3b) provides an exemplary mature form of FGF1 with mutations (K12V, L44F, M67I, L73V, N95V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 56 (FGF1(1-140αα)M3c) provides an exemplary mature form of FGF1 with mutations (K12V, L44F, M67I, L73V, N95V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 57 (FGF1^{ΔNT1} (1-140αα)M3) provides an exemplary N-terminally truncated form of FGF1 with mutations (L44F, M67I, L73V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 58 (FGF1^{ΔNT3} (1-140αα)M3) provides an exemplary N-terminally truncated form of FGF1 with mutations (L44F, M67I, L73V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 59 (FGF1^{ΔNT1} (1-140αα)M3a) provides an exemplary N-terminally truncated form of FGF1 with mutations (K12V, L44F, M67I, L73V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 60 (FGF1^{ΔNT3} (1-140αα)M3a) provides an exemplary N-terminally truncated form of FGF1 with mutations (K12V, L44F, M67I, L73V, A103G, V109L, L111I, C117V, R119G, Δ104-106, and Δ120-122 wherein numbering refers to SEQ ID NO: 5) for example to reduce mitogenic activity and increase thermostability.

SEQ ID NO: 61 (FGF1^{ΔNT1} (1-140αα)M3b) provides an exemplary N-terminally truncated form of FGF1 with mutations (L44F, M67I, L73V, N95V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 62 (FGF1^{ΔNT3} (1-140αα)M3b) provides an exemplary N-terminally truncated form of FGF1 with mutations (L44F, M67I, L73V, N95V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 63 (FGF1^{ΔNT1} (1-140αα)M3c) provides an exemplary N-terminally truncated form of FGF1 with mutations (K12V, L44F, M67I, L73V, N95V, V109L, L111I, C117V, A103G, R119G, Δ104-106, and Δ120-122 wherein numbering refers to SEQ ID NO: 5) for example to reduce mitogenic activity and increase thermostability.

SEQ ID NO: 64 (FGF1^{ΔNT3} (1-140αα)M3c) provides an exemplary N-terminally truncated form of FGF1 with point mutations K12V, L44F, M67I, L73V, N95V, V109L, L111I, C117V, A103G, R119G, Δ104-106, and Δ120-122, wherein numbering refers to SEQ ID NO: 5, for example to reduce mitogenic activity and increase thermostability.

SEQ ID NO: 65 (FGF1 (1-140αα) provides an exemplary mature form of FGF1 with point mutations K12V, N95V, and K118N, wherein numbering refers to SEQ ID NO: 5).

SEQ ID NO: 66 (FGF1 (1-140αα) provides an exemplary mature form of FGF1 with point mutations K12V, N95, and K118E, wherein numbering refers to SEQ ID NO: 5.

SEQ ID NO: 67 FGF1 (1-140αα) K12V, N95V, C117V provides an exemplary mature form of FGF1 with point mutations K12V, N95V, and C117V, wherein numbering refers to SEQ ID NO: 5.

SEQ ID NO: 68 (FGF1 (1-140αα) provides an exemplary mature form of FGF1 with point mutations K12V, N95V, C117V, and K118N, wherein numbering refers to SEQ ID NO: 5.

SEQ ID NO: 69 (FGF1 (1-140αα) provides an exemplary mature form of FGF1 with point mutations K12V, N95V, C117V, and K118E, wherein numbering refers to SEQ ID NO: 5.

SEQ ID NO: 70 (FGF1^{ΔNT} (10-140αα) provides an exemplary N-terminally truncated FGF1 with point mutations K12V and N95V, wherein numbering refers to SEQ ID NO: 5.

SEQ ID NO: 71 (FGF1^{ΔNT2} (12-140αα) provides an exemplary N-terminally truncated FGF1 with point mutations K12V and N95V, wherein numbering refers to SEQ ID NO: 5.

SEQ ID NO: 72 (FGF1^{ΔNT} (10-140αα) provides an exemplary N-terminally truncated FGF1 with a point mutation K12V, wherein numbering refers to SEQ ID NO: 5.

SEQ ID NO: 73 (FGF1^{ΔNT2} (12-140αα) provides an exemplary N-terminally truncated FGF1 with a point mutation K12V, wherein numbering refers to SEQ ID NO: 5.

SEQ ID NO: 74 (FGF1^{ΔNT} (10-140αα) provides an exemplary N-terminally truncated FGF1 with a point mutation N95V, wherein numbering refers to SEQ ID NO: 5.

SEQ ID NO: 75 (FGF1^{ΔNT2} (12-140αα) provides an exemplary N-terminally truncated FGF1 with a point mutation N95V, wherein numbering refers to SEQ ID NO: 5.

SEQ ID NO: 76 (FGF1^{ΔNT} (10-140αα) provides an exemplary N-terminally truncated FGF1 with point mutations K12V, N95V, and K118N, wherein numbering refers to SEQ ID NO: 5.

SEQ ID NO: 77 (FGF1^{ΔNT2} (12-140αα) provides an exemplary N-terminally truncated FGF1 with point mutations K12V, N95V, and K118E, wherein numbering refers to SEQ ID NO: 5.

SEQ ID NO: 78 (FGF1^{ΔNT} (10-140αα) provides an exemplary N-terminally truncated FGF1 with a point mutation K118N, wherein numbering refers to SEQ ID NO: 5.

SEQ ID NO: 79 (FGF1^{ΔNT2} (12-140αα) provides an exemplary N-terminally truncated FGF1 with a point mutation K118E, wherein numbering refers to SEQ ID NO: 5.

SEQ ID NO: 80 (FGF1 (1-140αα) provides an exemplary mature form of FGF1 with point mutations K9T and N10T, wherein numbering refers to SEQ ID NO: 5.

SEQ ID NO: 81 (FGF1 (1-140αα) provides an exemplary mature form of FGF1 with point mutations K9T, N10T, and N95V, wherein numbering refers to SEQ ID NO: 5.

SEQ ID NO: 82 (FGF1 (1-140αα) provides an exemplary mature form of FGF1 with point mutations K9T, N10T, and K118N, wherein numbering refers to SEQ ID NO: 5.

SEQ ID NO: 83 (FGF1 (1-140αα) provides an exemplary mature form of FGF1 with a mutant NLS sequence.

SEQ ID NO: 84 (FGF1^{ΔNT} (1-140αα) provides an exemplary N-terminally truncated form of FGF1 with point mutations Q40P and S47I, wherein numbering refers to SEQ ID NO: 5.

SEQ ID NO: 85 (FGF1^{ΔNT3} (1-140αα) provides an exemplary N-terminally truncated form of FGF1 with point mutations Q40P and S47I, wherein numbering refers to SEQ NO: 5.

SEQ ID NO: 86 (FGF1 (1-140αα) provides an exemplary mature form of FGF1 with point mutations K12V, Q40P, S47I, and N95V, wherein numbering refers to SEQ ID NO: 5.

SEQ ID NO: 87 FGF1^{ΔNT} (1-140αα) provides an exemplary N-terminally truncated form of FGF1 with point mutations K12V, Q40P, S47I, and N95V, wherein numbering refers to SEQ ID NO: 5.

SEQ ID NO: 88 (FGF1^{ΔNT3} (1-140αα) provides an exemplary N-terminally truncated form of FGF1 with point mutations K12V, Q40P, S47I, and N95V (wherein numbering refers to SEQ ID NO: 5).

SEQ ID NO: 89 (FGF1^{ΔNT} (1-140αα) provides an exemplary N-terminally truncated form of FGF1 with point mutations, Q40P, S47I, and H93G, wherein numbering refers to SEQ ID NO: 5.

SEQ ID NO: 90 (FGF1^{ΔNT3} (1-140αα) provides an exemplary N-terminally truncated form of FGF1 with point mutations Q40P, S47I, and H93G, wherein numbering refers to SEQ ID NO: 5.

SEQ ID NO: 91 (FGF1 (1-140αα) provides an exemplary mature form of FGF1 with point mutations K12V, Q40P, S47I, H93G, and N95V, wherein numbering refers to SEQ NO: 5.

SEQ ID NO: 92 (FGF1^{ΔNT} (1-140αα) provides an exemplary N-terminally truncated form of FGF1 with point mutations K12V, Q40P, S47I, H93G, and N95V, wherein numbering refers to SEQ ID NO: 5.

SEQ ID NO: 93 (FGF1^{ΔNT3} (1-140αα) provides an exemplary N-terminally truncated form of FGF1 with point mutations K12V, Q40P, S47I, H93G, and N95V, wherein numbering refers to SEQ ID NO: 5.

SEQ ID NO: 94 (FGF1^{ΔNT} (1-140αα) provides an exemplary N-terminally truncated form of FGF1 with point mutations C117P and K118V, wherein numbering refers to SEQ ID NO:).

SEQ ID NO: 95 (FGF1^{ΔNT3} (1-140αα) provides an exemplary N-terminally truncated form of FGF1 with point mutations C117P and K118V, wherein numbering refers to SEQ ID NO: 5.

SEQ ID NO: 96 (FGF1 (1-140αα) provides an exemplary mature form of FGF1 with point mutations K12V, N95V, C117P, and K118V, wherein numbering refers to SEQ ID NO: 5.

SEQ ID NO: 97 (FGF1 (1-140αα) provides an exemplary mature form of FGF1 with a point mutation R35E, wherein numbering refers to SEQ ID NO: 5.

SEQ ID NO: 98 provides an exemplary FGF1 heparan binding KKK mutant analog K112D, K113Q, K118V (wherein numbering refers to SEQ ID NO: 5).

SEQ ID NO: 99 provides an exemplary FGF1 heparan binding KKK mutant analog with mutations K112D, K113Q, C117V, K118V (wherein numbering refers to SEQ ID NO: 5).

SEQ ID NO: 100 provides an exemplary FGF1 heparan binding KKK mutant analog with an N-terminal truncation and mutations K112D, K113Q, K118V (wherein numbering refers to SEQ ID NO: 5).

SEQ ID NO: 101 provides an exemplary FGF1 heparan binding KKK mutant analog with an N-terminal truncation and mutations K112D, K113Q, K118V (wherein numbering refers to SEQ ID NO: 5).

SEQ ID NO: 102 provides an exemplary FGF1 heparan binding KKK mutant analog with an N-terminal truncation and mutations K112D, K113Q, C117V, K118V (wherein numbering refers to SEQ ID NO: 5).

SEQ ID NO: 103 provides an exemplary FGF1 heparan binding KKK mutant analog with an N-terminal truncation and mutations K112D, K113Q, C117V, K118V (wherein numbering refers to SEQ ID NO: 5).

SEQ ID NO: 104 provides an exemplary FGF1 heparan binding KKK mutant analog with mutations K12V, N95V, K112D, K113Q, K118V, wherein numbering refers to SEQ ID NO: 5.

SEQ ID NO: 105 provides an exemplary FGF1 heparan binding KKK mutant analog with mutations K12V, N95V, K112D, K113Q, C117V, K118V (wherein numbering refers to SEQ ID NO: 5).

SEQ ID NO: 106 (FGF1(1-140αα) R35E, C117V, KKK) provides an exemplary mature form of FGF1 with mutations (R35E, K112D, K113Q, C117V, and K118V, wherein numbering refers to SEQ ID NO: 5, for example to reduce mitogenic activity and increase thermostability.

SEQ ID NO: 107 (FGF1(1-140αα) R35E, C117V, K12V, N95V) provides an exemplary mature form of FGF1 with mutations K12V, R35E, N95V, and C117V, wherein numbering refers to SEQ ID NO: 5, for example to reduce mitogenic activity and increase thermostability.

SEQ ID NO: 108 (FGF1^{ΔNT1}(10-140αα) R35E, C117V) provides an exemplary N-terminally truncated form of FGF1 with mutations R35E and C117V, wherein numbering refers to SEQ ID NO: 5, for example to reduce mitogenic activity and increase thermostability.

SEQ ID NO: 109 (FGF1^{ΔNTKN} KKK (10-140αα)) provides an exemplary N-terminally truncated form of FGF1 with mutations (K112D, K113Q, K118V, K12V, N95V, C117V, and R35E, wherein numbering refers to SEQ ID NO: 5) to reduce mitogenic activity and increase thermostability.

SEQ ID NO: 110 (FGF1 KKK (KN) (1-140αα)) provides an exemplary mature form of FGF1 with mutations K112D, K113Q, K118V, K12V, N95V, C117V, and R35E, wherein numbering refers to SEQ ID NO: 5, for example to reduce mitogenic activity and increase thermostability.

SEQ ID NO: 111 (FGF1^{ΔNT1} (10-140αα) M2KN) provides an exemplary N-terminally truncated form of FGF1 with mutations K12V, L44F, R35E, C83T, N95V, C117V, and F132W, wherein numbering refers to SEQ ID NO: 5, for example to reduce mitogenic activity and increase thermostability.

SEQ ID NO: 112 (FGF1^{ΔNT1} (10-140αα) M2KNKKK) provides an exemplary N-terminally truncated form of FGF1 with mutations K12V, L44F, R35E, C83T, N95V, C117V, K112D, K113Q, K118V, and F132W, wherein numbering refers to SEQ ID NO: 5, for example to reduce mitogenic activity and increase thermostability.

SEQ ID NO: 113 (FGF1(1-140αα) R35V, C117V) provides an exemplary mature form of FGF1 with mutations R35V and C117V, wherein numbering refers to SEQ ID NO: 5, for example to reduce mitogenic activity and increase thermostability.

SEQ ID NO: 114 (FGF1(1-140αα) R35V, C117V, KKK) provides an exemplary mature form of FGF1 with mutations R35V, K112D, K113Q, C117V, and K118V, wherein numbering refers to SEQ ID NO: 5, for example to reduce mitogenic activity and increase thermostability.

SEQ ID NO: 115 (FGF1(1-140αα) K12V, R35V, N95V, C117V) provides an exemplary mature form of FGF1 with mutations K12V, R35V, N95V, and C117V (wherein numbering refers to SEQ ID NO: 5) for example to reduce mitogenic activity and increase thermostability.

SEQ ID NO: 116 (FGF1^{ΔNT1} (10-140αα) R35V, C117V) provides an exemplary N-terminally truncated form of FGF1 with mutations R35V and C117V (wherein numbering refers to SEQ ID NO: 5) for example to reduce mitogenic activity and increase thermostability.

SEQ ID NO: 117 (FGF1^{ΔNTKN} KKK (10-140αα)) provides an exemplary N-terminally truncated form of FGF1 with mutations K112D, K113Q, K118V K12V, N95V, C117V, and R35V (wherein numbering refers to SEQ ID NO: 5) for example to reduce mitogenic activity and increase thermostability.

SEQ ID NO: 118 (FGF1 KKK (KN) (1-140αα)) provides an exemplary mature form of FGF1 with mutations (K112D, K113Q, K118V, K12V, N95V, C117V, and R35V, wherein numbering refers to SEQ ID NO: 5) for example to reduce mitogenic activity and increase thermostability.

SEQ ID NO: 119 (FGF1^{ΔNT1} (10-140αα) M2KN) provides an exemplary N-terminally truncated form of FGF1 with mutations K12V, L44F, R35V, C83T, N95V, C117V, and F132W (wherein numbering refers to SEQ ID NO: 5) for example to reduce mitogenic activity and increase thermostability.

SEQ ID NO: 120 (FGF1^{ΔNT1} (10-140αα) M2KNKKK) provides an exemplary N-terminally truncated form of FGF1 with mutations K12V, L44F, R35V, C83T, N95V, C117V, K112D, K113Q, K118V, and F132W (wherein numbering refers to SEQ ID NO: 5) for example to reduce mitogenic activity and increase thermostability.

SEQ ID NO: 121 (FGF1-140αα) C117V, KKKR provides an exemplary mature form of FGF1 with mutations K112D, K113Q, C117V, K118V, and R119V (wherein numbering refers to SEQ ID NO: 5) for example to reduce mitogenic activity and increase thermostability.

SEQ ID NO: 122 (FGF1-140αα) C117V, KY provides an exemplary mature form of FGF1 with mutations K12V, Y94V, and C117V (wherein numbering refers to SEQ ID NO: 5) for example to reduce mitogenic activity and increase thermostability.

SEQ ID NO: 123 (FGF1-140αα) C117V, KE provides an exemplary mature form of FGF1 with mutations (K12V, E87V, C117V, wherein numbering refers to SEQ ID NO: 5) for example to reduce mitogenic activity and increase thermostability.

SEQ ID NO: 124 (FGF1-140αα) C117V, KEY provides an exemplary mature form of FGF1 with mutations K12V, E87V, Y94V, and C117V (wherein numbering refers to SEQ ID NO: 5) for example to reduce mitogenic activity and increase thermostability.

SEQ ID NO: 125 (FGF1-140αα) C117V, KNY provides an exemplary mature form of FGF1 with mutations K12V, Y94V, N95V, and C117V (wherein numbering refers to SEQ ID NO: 5) for example to reduce mitogenic activity and increase thermostability.

SEQ ID NO: 126 (FGF1-140αα) K12V, L46V, E87V, N95V, C117V, P134V provides an exemplary mature form of FGF1 with point mutations K12V, L46V, E87V, N95V, C117V, and P134V (wherein numbering refers to SEQ ID NO: 5) for example to reduce mitogenic activity and increase thermostability.

SEQ ID NO: 127 (FGF1-140αα) C117V, K118V provides an exemplary mature form of FGF1 with mutations C117V and K118V (wherein numbering refers to SEQ ID NO: 5) for example to reduce mitogenic activity and increase thermostability.

SEQ ID NO: 128 (FGF^{ΔNT1C} 10-140αα) K12V, N95V, C83T, C117V provides an exemplary N-terminally truncated form of FGF1 with mutations K12V, N95V, C83T, and C117V (wherein numbering refers to SEQ ID NO: 5) for example to reduce mitogenic activity and increase thermostability.

SEQ ID NO: 129 (FGF^{ΔNT1C} 10-140αα) K12V, N95V, C16T, C83S, C117A, provides an exemplary N-terminally truncated form of FGF1 with mutations K12V, N95V, C16T, C83S, and C117A (wherein numbering refers to SEQ ID NO: 5) for example for example to reduce mitogenic activity and increase thermostability.

SEQ ID NO: 130 (FGF^{ΔNT1} 10-140αα) H21Y, L44F, H102Y, F108Y, C117V, provides an exemplary N-terminally truncated form of FGF1 with mutations H21Y, L44F, H102Y, F108Y, and C117V (wherein numbering refers to SEQ ID NO: 5) for example to reduce mitogenic activity and increase thermostability.

SEQ ID NO: 131 (FGF^{ΔNT1} 10-140αα) K12V, H21Y, L44F, N95V, H102Y, F108Y, C117V, provides an exemplary N-terminally truncated form of FGF1 with mutations K12V, H21Y, L44F, N95V, H102Y, F108Y, and C117V (wherein numbering refers to SEQ ID NO: 5) for example to reduce mitogenic activity and increase thermostability.

SEQ ID NO: 132 (FGF1 1-140αα) K12V, H21Y, L44F, N95V, H102Y, F108Y, C117V, provides an exemplary mature form of FGF1 with mutations K12V, H21Y, L44F, N95V, H102Y, F108Y, and C117V (wherein numbering refers to SEQ ID NO: 5) for example to reduce mitogenic activity and increase thermostability.

SEQ ID NO: 133 (FGF^{ΔNT1C} 10-140αα) K12V, N95V, C117V, provides an exemplary N-terminally truncated form of FGF1 with mutations K12V, N95V, and C117V (wherein numbering refers to SEQ ID NO: 5) for example to reduce the mitogenicity and increase the stability of FGF1.

SEQ ID NO: 134 (FGF1 KKK 1-140αα) K112D, K113Q, K118V, provides an exemplary mature form of FGF1 with mutations K112D, K113Q, and K118V (wherein numbering refers to SEQ ID NO: 5) for example to reduce the heparan binding affinity of FGF1.

SEQ ID NO: 135 (FGF1 1-140αα) K12V, Q40P, S47I, H93G, N95V, provides an exemplary mature form of FGF1 with mutations K12V, Q40P, S47I, H93G, and N95V (wherein numbering refers to SEQ ID NO: 5) for example to reduce the mitogenicity and increase the thermal stability of FGF1.

SEQ ID NO: 136 (FGF^{ΔNT} 10-140αα) K12V, Q40P, S47I, H93G, N95V provides an exemplary N-terminally truncated form of FGF1 with mutations K12V, Q40P, S47I, H93G, and N95V (wherein numbering refers to SEQ ID NO: 5) for example to reduce the mitogenicity and increase the thermal stability of FGF1.

SEQ ID NO: 137 (FGF1 1-140αα) M2KN K12V, L44F, C83T, N95V, C117V, F132W provides an exemplary mature form of FGF1 with mutations K12V, L44F, C83T, N95V, C117V, and F132W (wherein numbering refers to SEQ ID NO: 5) for example to reduce the mitogenicity without increasing the thermal stability of FGF1.

SEQ ID NO: 138 (FGF1 1-140αα) C117V provides an exemplary mature form of FGF1 with mutation C117V, wherein numbering refers to SEQ ID NO: 5, for example to improve the stability of FGF1 by eliminating a free cysteine the can form disulfide brigded aggregated protein.

SEQ ID NO: 139 (FGF1 1-140αα) KKK(KN) K112D, K113Q, K118V, K12V, N95V, C117V provides an exemplary mature form of FGF1 with mutations K112D, K113Q, K118V, K12V, N95V, and C117V (wherein numbering refers to SEQ ID NO: 5) for example to reduce mitogenicity and heparan binding, and decrease the potential for protein aggregation of FGF1.

SEQ ID NO: 140 (FGF1 10-140αα) M2KN K12V, L44F, C83T, N95V, C117V, F132W, provides an exemplary N-terminally truncated form of FGF1 with mutations (K12V, L44F, C83T, N95V, C117V, and F132W (wherein numbering refers to SEQ ID NO: 5) for example to reduce mitogenicity and decrease the potential for protein aggregation of FGF1, without affecting the thermal stability.

SEQ ID NO: 141 (FGF1 1-140αα) R35E, C117V, provides an exemplary mature form of FGF1 with mutations R35E and C117V (wherein numbering refers to SEQ ID NO: 5) for example to manipulate the receptor binding affinity/specificity and decrease the potential for protein aggregation of FGF1.

SEQ ID NO: 142 (FGF1 1-140αα) KY K12V, Y94V, C117V, provides an exemplary mature form of FGF1 with mutations K12V, Y94V, and C117V (wherein numbering refers to SEQ ID NO: 5) for example to manipulate the receptor binding affinity/specificity and decrease the potential for protein aggregation of FGF1.

SEQ ID NO: 143 (FGF1 1-140αα) KE K12V, E87V, C117V, provides an exemplary mature form of FGF1 with mutations K12V, E87V, and C117V (wherein numbering refers to SEQ ID NO: 5) for example to manipulate the receptor binding affinity/specificity and decrease the potential for protein aggregation of FGF1

SEQ ID NO: 144 (FGF1 1-140αα) KKKR K112D, K113Q, C117V, K118V, R119V provides an exemplary mature form of FGF1 with mutations K112D, K113Q, C117V, K118V, and R119V (wherein numbering refers to SEQ ID NO: 5) for example to reduce the heparan binding affinity/specificity and decrease the potential for protein aggregation of FGF1.

SEQ ID NO: 145 (FGF1 1-140αα) KN R35E, K12V, N95V, C117V provides an exemplary mature form of FGF1 with mutations R35E, K12V, N95V, and C117V (wherein numbering refers to SEQ ID NO: 5) for example to manipulate the receptor binding affinity/specificity and decrease the potential for protein aggregation of FGF1.

SEQ ID NO: 146 (FGF1 10-140αα) KN R35E, C117V provides an exemplary N-terminally truncated form of FGF1 with mutations R35E and C117V (wherein numbering refers to SEQ ID NO: 5) for example to manipulate the receptor binding affinity/specificity and decrease the potential for protein aggregation of FGF1.

SEQ ID NO: 147 provides an exemplary mature form of FGF1 with point mutations H21Y, L44F, H102Y, and F108Y, wherein numbering refers to SEQ ID NO: 5.

SEQ ID NO: 148 provides an exemplary N-terminally truncated form of FGF1 with point mutations H21Y, L44F, H102Y, and F108Y (wherein numbering refers to SEQ ID NO: 5).

SEQ ID NO: 149 provides an exemplary mature form of FGF1 with point mutations K12V, H21Y, L44F, N95V, H102Y, and F108Y (wherein numbering refers to SEQ ID NO: 5).

SEQ ID NO: 150 provides an exemplary N-terminally truncated form of FGF1 with point mutations K12V, H21Y, L44F, N95V, H102Y, F108Y, and C117V (wherein numbering refers to SEQ ID NO: 5).

SEQ ID NO: 151 provides an exemplary mature form of FGF1 with point mutations H21Y, L44F, H102Y, F108Y, and C117V (wherein numbering refers to SEQ ID NO: 5).

SEQ ID NO: 152 provides an exemplary mature form of FGF1 with point mutations H21Y, L44F, A66C, H102Y, and F108Y (wherein numbering refers to SEQ ID NO: 5).

SEQ ID NO: 153 provides an exemplary mature form of FGF1 with six point mutations (H21Y, R35E, L44F, H102Y, F108Y, and C117V, wherein numbering refers to SEQ ID NO: 5).

SEQ ID NO: 154 provides an exemplary mature form of FGF1 with seven point mutations (K12V, H21Y, L44F, Y94V, H102Y, F108Y, and C117V, wherein numbering refers to SEQ ID NO: 5).

SEQ ID NO: 155 provides an exemplary mature form of FGF1 with point mutation N18R, wherein numbering refers to SEQ ID NO: 5.

SEQ ID NO: 156 provides an exemplary N-terminally truncated form of FGF1 with point mutation N18R, wherein numbering refers to SEQ ID NO: 5.

SEQ ID NO: 157 provides an exemplary mature form of FGF1 with point mutations K12V, N18R, and N95V, wherein numbering refers to SEQ ID NO: 5.

SEQ ID NO: 158 provides an exemplary N-terminally truncated form of FGF1 with point mutations K12V, N18R, N95V, and C117V, wherein numbering refers to SEQ ID NO: 5.

SEQ ID NO: 159 provides an exemplary mature form of FGF1 with point mutations N18R, and C117V, wherein numbering refers to SEQ ID NO: 5.

SEQ ID NO: 160 provides an exemplary mature form of FGF1 with point mutations N18R, and A66C, wherein numbering refers to SEQ ID NO: 5.

SEQ ID NO: 161 provides an exemplary mature form of FGF1 with point mutations (N18R, R35E, and C117V, wherein numbering refers to SEQ ID NO: 5.

SEQ ID NO: 162 provides an exemplary mature form of FGF1 with point mutations K12V, N18R, Y94V, and C117V, wherein numbering refers to SEQ ID NO: 5.

SEQ ID NO: 163 provides an exemplary mature form of FGF1 with point mutation N18K, wherein numbering refers to SEQ ID NO: 5.

SEQ ID NO: 164 provides an exemplary N-terminally truncated form of FGF1 with point mutation N18K, wherein numbering refers to SEQ ID NO: 5.

SEQ ID NO: 165 provides an exemplary mature form of FGF1 with point mutations K12V, N18K, and N95V, wherein numbering refers to SEQ ID NO: 5).

SEQ ID NO: 166 provides an exemplary N-terminally truncated form of FGF1 with point mutations K12V, N18K, N95V, and C117V, wherein numbering refers to SEQ ID NO: 5.

SEQ ID NO: 167 provides an exemplary mature form of FGF1 with point mutations N18K, and C117V, wherein numbering refers to SEQ ID NO: 5.

SEQ ID NO: 168 provides an exemplary mature form of FGF1 with point mutations N18K, and A66C, wherein numbering refers to SEQ ID NO: 5.

SEQ ID NO: 169 provides an exemplary mature form of FGF1 with point mutations N18K, R35E, and C117V, wherein numbering refers to SEQ ID NO: 5.

SEQ ID NO: 170 provides an exemplary mature form of FGF1 with point mutations K12V, N18K, Y94V, and C117V, wherein numbering refers to SEQ ID NO: 5.

SEQ ID NO: 171 provides an exemplary mature form of FGF1 with point mutation N114R, wherein numbering refers to SEQ ID NO: 5.

SEQ ID NO: 172 provides an exemplary N-terminally truncated form of FGF1 with point mutation N114R, wherein numbering refers to SEQ ID NO: 5.

SEQ ID NO: 173 provides an exemplary mature form of FGF1 with point mutations K12V, N114R and N95V, wherein numbering refers to SEQ ID NO: 5.

SEQ ID NO: 174 provides an exemplary N-terminally truncated form of FGF1 with point mutations K12V, N95V, N114R and C117V, wherein numbering refers to SEQ ID NO: 5.

SEQ ID NO: 175 provides an exemplary mature form of FGF1 with point mutations (N114R and C117V, wherein numbering refers to SEQ ID NO: 5.

SEQ ID NO: 176 provides an exemplary mature form of FGF1 with point mutations N114R and A66C, wherein numbering refers to SEQ ID NO: 5.

SEQ ID NO: 177 provides an exemplary mature form of FGF1 with point mutations R35E, N114R and C117V, wherein numbering refers to SEQ ID NO: 5.

SEQ ID NO: 178 provides an exemplary mature form of FGF1 with point mutations K12V, Y94V, N114R and C117V, wherein numbering refers to SEQ ID NO: 5.

SEQ ID NO: 179 provides an exemplary mature form of FGF1 with point mutation (N114K, wherein numbering refers to SEQ ID NO: 5).

SEQ ID NO: 180 provides an exemplary N-terminally truncated form of FGF1 with point mutation (N114K, wherein numbering refers to SEQ ID NO: 5).

SEQ ID NO: 181 provides an exemplary mature form of FGF1 with point mutations (K12V, N114K and N95V, wherein numbering refers to SEQ ID NO: 5).

SEQ ID NO: 182 provides an exemplary N-terminally truncated form of FGF1 with point mutations K12V, N95V, N114K and C117V, wherein numbering refers to SEQ ID NO: 5.

SEQ ID NO: 183 provides an exemplary mature form of FGF1 with point mutations N114K and C117V, wherein numbering refers to SEQ ID NO: 5.

SEQ ID NO: 184 provides an exemplary mature form of FGF1 with point mutations N114K and A66C, wherein numbering refers to SEQ ID NO: 5.

SEQ ID NO: 185 provides an exemplary mature form of FGF1 with point mutations R35E, N114K and C117V, wherein numbering refers to SEQ ID NO: 5.

SEQ ID NO: 186 provides an exemplary mature form of FGF1 with point mutations K12V, Y94V, N114K and C117V, wherein numbering refers to SEQ ID NO: 5.

SEQ ID NO: 187 provides an exemplary mature form of FGF1 with point mutation S17R, wherein numbering refers to SEQ ID NO: 5.

SEQ ID NO: 188 provides an exemplary N-terminally truncated form of FGF1 with point mutation S 17R, wherein numbering refers to SEQ ID NO: 5.

SEQ ID NO: 189 provides an exemplary mature form of FGF1 with point mutations K12V, S17R and N95V, wherein numbering refers to SEQ ID NO: 5.

SEQ ID NO: 190 provides an exemplary N-terminally truncated form of FGF1 with point mutations K12V, N95V, S17R and C117V, wherein numbering refers to SEQ ID NO: 5.

SEQ ID NO: 191 provides an exemplary mature form of FGF1 with point mutations S17R and C117V, wherein numbering refers to SEQ ID NO: 5.

SEQ ID NO: 192 provides an exemplary mature form of FGF1 with point mutations S17R and A66C, wherein numbering refers to SEQ ID NO: 5.

SEQ ID NO: 193 provides an exemplary mature form of FGF1 with point mutations R35E, S17R and C117V, wherein numbering refers to SEQ ID NO: 5.

SEQ ID NO: 194 provides an exemplary mature form of FGF1 with point mutations K12V, Y94V, S17R and C117V, wherein numbering refers to SEQ ID NO: 5.

SEQ ID NO: 195 provides an exemplary mature form of FGF1 with point mutation S 17K, wherein numbering refers to SEQ ID NO: 5.

SEQ ID NO: 196 provides an exemplary N-terminally truncated form of FGF1 with point mutation S 17K, wherein numbering refers to SEQ ID NO: 5.

SEQ ID NO: 197 provides an exemplary mature form of FGF1 with point mutations K12V, S17K and N95V, wherein numbering refers to SEQ ID NO: 5.

SEQ ID NO: 198 provides an exemplary N-terminally truncated form of FGF1 with point mutations K12V, N95V, S17K and C117V, wherein numbering refers to SEQ ID NO: 5.

SEQ ID NO: 199 provides an exemplary mature form of FGF1 with point mutations S17K and C117V, wherein numbering refers to SEQ ID NO: 5.

SEQ ID NO: 200 provides an exemplary mature form of FGF1 with point mutations S17K and A66C, wherein numbering refers to SEQ ID NO: 5.

SEQ ID NO: 201 provides an exemplary mature form of FGF1 with point mutations R35E, S17K and C117V, wherein numbering refers to SEQ ID NO: 5.

SEQ ID NO: 202 provides an exemplary mature form of FGF1 with point mutations K12V, Y94V, S17K and C117V, wherein numbering refers to SEQ ID NO: 5.

SEQ ID NO: 203 provides an exemplary mature form of FGF1 with point mutation Q127R, wherein numbering refers to SEQ ID NO: 5.

SEQ ID NO: 204 provides an exemplary N-terminally truncated form of FGF1 with point mutation Q127R, wherein numbering refers to SEQ ID NO: 5.

SEQ ID NO: 205 provides an exemplary mature form of FGF1 with point mutations K12V, Q127R and N95V, wherein numbering refers to SEQ ID NO: 5.

SEQ ID NO: 206 provides an exemplary N-terminally truncated form of FGF1 with point mutations K12V, N95V, Q127R and C117V, wherein numbering refers to SEQ ID NO: 5.

SEQ ID NO: 207 provides an exemplary mature form of FGF1 with point mutations Q127R and C117V, wherein numbering refers to SEQ ID NO: 5.

SEQ ID NO: 208 provides an exemplary mature form of FGF1 with point mutations Q127R and A66C, wherein numbering refers to SEQ ID NO: 5.

SEQ ID NO: 209 provides an exemplary mature form of FGF1 with point mutations R35E, Q127R and C117V, wherein numbering refers to SEQ ID NO: 5.

SEQ ID NO: 210 provides an exemplary mature form of FGF1 with point mutations K12V, Y94V, Q127R and C117V, wherein numbering refers to SEQ ID NO: 5.

SEQ ID NO: 211 provides an exemplary mature form of FGF1 with point mutation Q127K, wherein numbering refers to SEQ ID NO: 5.

SEQ ID NO: 212 provides an exemplary N-terminally truncated form of FGF1 with point mutation Q127K, wherein numbering refers to SEQ ID NO: 5.

SEQ ID NO: 213 provides an exemplary mature form of FGF1 with point mutations K12V, Q127K and N95V, wherein numbering refers to SEQ ID NO: 5.

SEQ ID NO: 214 provides an exemplary N-terminally truncated form of FGF1 with point mutations K12V, N95V, Q127K and C117V, wherein numbering refers to SEQ ID NO: 5.

SEQ ID NO: 215 provides an exemplary mature form of FGF1 with point mutations Q127K and C117V, wherein numbering refers to SEQ ID NO: 5.

SEQ ID NO: 216 provides an exemplary mature form of FGF1 with point mutations Q127K and A66C, wherein numbering refers to SEQ ID NO: 5.

SEQ ID NO: 217 provides an exemplary mature form of FGF1 with point mutations R35E, Q127K and C117V, wherein numbering refers to SEQ ID NO: 5.

SEQ ID NO: 218 provides an exemplary mature form of FGF1 with point mutations K12V, Y94V, Q127K and C117V, wherein numbering refers to SEQ ID NO: 5.

SEQ ID NO: 219 provides an exemplary mature form of FGF1 with point mutation E49D, wherein numbering refers to SEQ ID NO: 5.

SEQ ID NO: 220 provides an exemplary N-terminally truncated form of FGF1 with point mutation E49D, wherein numbering refers to SEQ ID NO: 5.

SEQ ID NO: 221 provides an exemplary mature form of FGF1 with point mutations K12V, E49D and N95V, wherein numbering refers to SEQ ID NO: 5.

SEQ ID NO: 222 provides an exemplary N-terminally truncated form of FGF1 with point mutations K12V, N95V, E49D and C117V, wherein numbering refers to SEQ ID NO: 5.

SEQ ID NO: 223 provides an exemplary mature form of FGF1 with point mutations E49D and C117V, wherein numbering refers to SEQ ID NO: 5.

SEQ ID NO: 224 provides an exemplary mature form of FGF1 with point mutations E49D and A66C, wherein numbering refers to SEQ ID NO: 5.

SEQ ID NO: 225 provides an exemplary mature form of FGF1 with point mutations R35E, E49D and C117V, wherein numbering refers to SEQ ID NO: 5.

SEQ ID NO: 226 provides an exemplary mature form of FGF1 with point mutations K12V, Y94V, E49Dand C117V, wherein numbering refers to SEQ ID NO: 5.

SEQ ID NO: 227 provides an exemplary mature form of FGF1 with point mutation E49K, wherein numbering refers to SEQ ID NO: 5.

SEQ ID NO: 228 provides an exemplary N-terminally truncated form of FGF1 with point mutation E49K, wherein numbering refers to SEQ ID NO: 5.

SEQ ID NO: 229 provides an exemplary mature form of FGF1 with point mutations K12V, E49K and N95V, wherein numbering refers to SEQ ID NO: 5.

SEQ ID NO: 230 provides an exemplary N-terminally truncated form of FGF1 with point mutations K12V, N95V, E49K and C117V, wherein numbering refers to SEQ ID NO: 5.

SEQ ID NO: 231 provides an exemplary mature form of FGF1 with point mutations E49K and C117V, wherein numbering refers to SEQ ID NO: 5.

SEQ ID NO: 232 provides an exemplary mature form of FGF1 with point mutations E49K and A66C, wherein numbering refers to SEQ ID NO: 5.

SEQ ID NO: 233 provides an exemplary mature form of FGF1 with point mutations R35E, E49K and C117V, wherein numbering refers to SEQ ID NO: 5.

SEQ ID NO: 234 provides an exemplary mature form of FGF1 with point mutations K12V, Y94V, E49K and C117V, wherein numbering refers to SEQ ID NO: 5.

SEQ ID NO: 235 provides an exemplary mature form of FGF1 with point mutation Y55F, wherein numbering refers to SEQ ID NO: 5.

SEQ ID NO: 236 provides an exemplary N-terminally truncated form of FGF1 with point mutation Y55F, wherein numbering refers to SEQ ID NO: 5.

SEQ ID NO: 237 provides an exemplary mature form of FGF1 with point mutations K12V, Y55F and N95V, wherein numbering refers to SEQ ID NO: 5.

SEQ ID NO: 238 provides an exemplary N-terminally truncated form of FGF1 with point mutations K12V, N95V, Y55F and C117V, wherein numbering refers to SEQ ID NO: 5.

SEQ ID NO: 239 provides an exemplary mature form of FGF1 with point mutations Y55F and C117V, wherein numbering refers to SEQ ID NO: 5.

SEQ ID NO: 240 provides an exemplary mature form of FGF1 with point mutations Y55F and A66C, wherein numbering refers to SEQ ID NO: 5.

SEQ ID NO: 241 provides an exemplary mature form of FGF1 with point mutations R35E, Y55F and C117V, wherein numbering refers to SEQ ID NO: 5.

SEQ ID NO: 242 provides an exemplary mature form of FGF1 with point mutations K12V, Y94V, Y55F and C117V, wherein numbering refers to SEQ ID NO: 5.

SEQ ID NO: 243 provides an exemplary mature form of FGF1 with point mutation Y55V, wherein numbering refers to SEQ ID NO: 5.

SEQ ID NO: 244 provides an exemplary N-terminally truncated form of FGF1 with point mutation Y55V, wherein numbering refers to SEQ ID NO: 5.

SEQ ID NO: 245 provides an exemplary mature form of FGF1 with point mutations K12V, Y55V and N95V, wherein numbering refers to SEQ ID NO: 5.

SEQ ID NO: 246 provides an exemplary N-terminally truncated form of FGF1 with point mutations K12V, N95V, Y55V and C117V, wherein numbering refers to SEQ ID NO: 5.

SEQ ID NO: 247 provides an exemplary mature form of FGF1 with point mutations Y55V and C117V, wherein numbering refers to SEQ ID NO: 5.

SEQ ID NO: 248 provides an exemplary mature form of FGF1 with point mutations Y55V and A66C, wherein numbering refers to SEQ ID NO: 5.

SEQ ID NO: 249 provides an exemplary mature form of FGF1 with point mutations R35E, Y55V and C117V, wherein numbering refers to SEQ ID NO: 5.

SEQ ID NO: 250 provides an exemplary mature form of FGF1 with point mutations K12V, Y94V, Y55V and C117V, wherein numbering refers to SEQ ID NO: 5.

SEQ ID NO: 251 provides an exemplary mature form of FGF1 with point mutation R88L, wherein numbering refers to SEQ ID NO: 5.

SEQ ID NO: 252 provides an exemplary N-terminally truncated form of FGF1 with point mutation R88L, wherein numbering refers to SEQ ID NO: 5.

SEQ ID NO: 253 provides an exemplary mature form of FGF1 with point mutations K12V, R88L and N95V, wherein numbering refers to SEQ ID NO: 5.

SEQ ID NO: 254 provides an exemplary N-terminally truncated form of FGF1 with point mutations K12V, N95V, R88L and C117V, wherein numbering refers to SEQ ID NO: 5.

SEQ ID NO: 255 provides an exemplary mature form of FGF1 with point mutations R88L and C117V, wherein numbering refers to SEQ ID NO: 5.

SEQ ID NO: 256 provides an exemplary mature form of FGF1 with point mutations R88L and A66C, wherein numbering refers to SEQ ID NO: 5.

SEQ ID NO: 257 provides an exemplary mature form of FGF1 with point mutations R35E, R88L and C117V, wherein numbering refers to SEQ ID NO: 5.

SEQ ID NO: 258 provides an exemplary mature form of FGF1 with point mutations K12V, Y94V, R88L and C117V, wherein numbering refers to SEQ ID NO: 5.

SEQ ID NO: 259 provides an exemplary mature form of FGF1 with point mutation R88Y, wherein numbering refers to SEQ ID NO: 5.

SEQ ID NO: 260 provides an exemplary N-terminally truncated form of FGF1 with point mutation R88Y, wherein numbering refers to SEQ ID NO: 5.

SEQ ID NO: 261 provides an exemplary mature form of FGF1 with point mutations K12V, R88Y and N95V, wherein numbering refers to SEQ ID NO: 5.

SEQ ID NO: 262 provides an exemplary N-terminally truncated form of FGF1 with point mutations K12V, N95V, R88Y and C117V, wherein numbering refers to SEQ ID NO: 5.

SEQ ID NO: 263 provides an exemplary mature form of FGF1 with point mutations R88Y and C117V, wherein numbering refers to SEQ ID NO: 5.

SEQ ID NO: 264 provides an exemplary mature form of FGF1 with point mutations R88Y and A66C, wherein numbering refers to SEQ ID NO: 5.

SEQ ID NO: 265 provides an exemplary mature form of FGF1 with point mutations R35E, R88Y and C117V, wherein numbering refers to SEQ ID NO: 5.

SEQ ID NO: 266 provides an exemplary mature form of FGF1 with point mutations K12V, Y94V, R88Y and C117V, wherein numbering refers to SEQ ID NO: 5.

SEQ ID NO: 267 provides an exemplary mature form of FGF1 with point mutation R88D, wherein numbering refers to SEQ ID NO: 5.

SEQ ID NO: 268 provides an exemplary N-terminally truncated form of FGF1 with point mutation R88D, wherein numbering refers to SEQ ID NO: 5.

SEQ ID NO: 269 provides an exemplary mature form of FGF1 with point mutations K12V, R88D and N95V, wherein numbering refers to SEQ ID NO: 5.

SEQ ID NO: 270 provides an exemplary N-terminally truncated form of FGF1 with point mutations K12V, N95V, R88D and C117V, wherein numbering refers to SEQ ID NO: 5.

SEQ ID NO: 271 provides an exemplary mature form of FGF1 with point mutations R88D and C117V, wherein numbering refers to SEQ ID NO: 5.

SEQ ID NO: 272 provides an exemplary mature form of FGF1 with point mutations R88D and A66C, wherein numbering refers to SEQ ID NO: 5.

SEQ ID NO: 273 provides an exemplary mature form of FGF1 with point mutations R35E, R88D and C117V, wherein numbering refers to SEQ ID NO: 5.

SEQ ID NO: 274 provides an exemplary mature form of FGF1 with point mutations K12V, Y94V, R88D and C117V, wherein numbering refers to SEQ ID NO: 5.

SEQ ID NO: 275 provides an exemplary mature form of FGF1 with point mutation Q43K, wherein numbering refers to SEQ ID NO: 5.

SEQ ID NO: 276 provides an exemplary N-terminally truncated form of FGF1 with point mutation Q43K, wherein numbering refers to SEQ ID NO: 5.

SEQ ID NO: 277 provides an exemplary mature form of FGF1 with point mutations K12V, Q43K and N95V, wherein numbering refers to SEQ ID NO: 5.

SEQ ID NO: 278 provides an exemplary N-terminally truncated form of FGF1 with point mutations K12V, N95V, Q43K and C117V, wherein numbering refers to SEQ ID NO: 5.

SEQ ID NO: 279 provides an exemplary mature form of FGF1 with point mutations Q43K and C117V, wherein numbering refers to SEQ ID NO: 5.

SEQ ID NO: 280 provides an exemplary mature form of FGF1 with point mutations Q43K and A66C, wherein numbering refers to SEQ ID NO: 5.

SEQ ID NO: 281 provides an exemplary mature form of FGF1 with point mutations R35E, Q43K and C117V, wherein numbering refers to SEQ ID NO: 5.

SEQ ID NO: 282 provides an exemplary mature form of FGF1 with point mutations K12V, Y94V, Q43K and C117V, wherein numbering refers to SEQ ID NO: 5).

SEQ ID NO: 283 provides an exemplary mature form of FGF1 with point mutation Q43A, wherein numbering refers to SEQ ID NO: 5.

SEQ ID NO: 284 provides an exemplary N-terminally truncated form of FGF1 with point mutation Q43A, wherein numbering refers to SEQ ID NO: 5.

SEQ ID NO: 285 provides an exemplary mature form of FGF1 with point mutations K12V, Q43A and N95V, wherein numbering refers to SEQ ID NO: 5.

SEQ ID NO: 286 provides an exemplary N-terminally truncated form of FGF1 with point mutations K12V, N95V, Q43A and C117V, wherein numbering refers to SEQ ID NO: 5.

SEQ ID NO: 287 provides an exemplary mature form of FGF1 with point mutations Q43A and C117V, wherein numbering refers to SEQ ID NO: 5.

SEQ ID NO: 288 provides an exemplary mature form of FGF1 with point mutations Q43A and A66C, wherein numbering refers to SEQ ID NO: 5.

SEQ ID NO: 289 provides an exemplary mature form of FGF1 with point mutations R35E, Q43A and C117V, wherein numbering refers to SEQ ID NO: 5.

SEQ ID NO: 290 provides an exemplary mature form of FGF1 with point mutations K12V, Y94V, Q43A and C117V, wherein numbering refers to SEQ ID NO: 5.

SEQ ID NO: 291 provides an exemplary mature form of FGF1 with point mutation Q43E, wherein numbering refers to SEQ ID NO: 5.

SEQ ID NO: 292 provides an exemplary N-terminally truncated form of FGF1 with point mutation Q43E, wherein numbering refers to SEQ ID NO: 5.

SEQ ID NO: 293 provides an exemplary mature form of FGF1 with point mutations K12V, Q43E and N95V, wherein numbering refers to SEQ ID NO: 5.

SEQ ID NO: 294 provides an exemplary N-terminally truncated form of FGF1 with point mutations K12V, N95V, Q43E and C117V, wherein numbering refers to SEQ ID NO: 5.

SEQ ID NO: 295 provides an exemplary mature form of FGF1 with point mutations Q43E and C117V, wherein numbering refers to SEQ ID NO: 5.

SEQ ID NO: 296 provides an exemplary mature form of FGF1 with point mutations Q43E and A66C, wherein numbering refers to SEQ ID NO: 5.

SEQ ID NO: 297 provides an exemplary mature form of FGF1 with point mutations R35E, Q43E and C117V, wherein numbering refers to SEQ ID NO: 5.

SEQ ID NO: 298 provides an exemplary mature form of FGF1 with point mutations K12V, Y94V, Q43E and C117V, wherein numbering refers to SEQ ID NO: 5.

SEQ ID NO: 299 provides an exemplary mature form of FGF1 with point mutation S47A, wherein numbering refers to SEQ ID NO: 5.

SEQ ID NO: 300 provides an exemplary N-terminally truncated form of FGF1 with point mutation S47A, wherein numbering refers to SEQ ID NO: 5.

SEQ ID NO: 301 provides an exemplary mature form of FGF1 with point mutations K12V, S47A and N95V, wherein numbering refers to SEQ ID NO: 5.

SEQ ID NO: 302 provides an exemplary N-terminally truncated form of FGF1 with point mutations K12V, N95V, S47A and C117V, wherein numbering refers to SEQ ID NO: 5.

SEQ ID NO: 303 provides an exemplary mature form of FGF1 with point mutations S47A and C117V, wherein numbering refers to SEQ ID NO: 5.

SEQ ID NO: 304 provides an exemplary mature form of FGF1 with point mutations S47A and A66C, wherein numbering refers to SEQ ID NO: 5.

SEQ ID NO: 305 provides an exemplary mature form of FGF1 with point mutations R35E, S47A and C117V, wherein numbering refers to SEQ ID NO: 5.

SEQ ID NO: 306 provides an exemplary mature form of FGF1 with point mutations K12V, Y94V, S47A and C117V, wherein numbering refers to SEQ ID NO: 5.

SEQ ID NO: 307 provides an exemplary mature form of FGF1 with point mutation S47V, wherein numbering refers to SEQ ID NO: 5.

SEQ ID NO: 308 provides an exemplary N-terminally truncated form of FGF1 with point mutation S47V, wherein numbering refers to SEQ ID NO: 5.

SEQ ID NO: 309 provides an exemplary mature form of FGF1 with point mutations K12V, S47V and N95V, wherein numbering refers to SEQ ID NO: 5.

SEQ ID NO: 310 provides an exemplary N-terminally truncated form of FGF1 with point mutations K12V, N95V, S47V and C117V, wherein numbering refers to SEQ ID NO: 5.

SEQ ID NO: 311 provides an exemplary mature form of FGF1 with point mutations S47V and C117V, wherein numbering refers to SEQ ID NO: 5.

SEQ ID NO: 312 provides an exemplary mature form of FGF1 with point mutations S47V and A66C, wherein numbering refers to SEQ ID NO: 5.

SEQ ID NO: 313 provides an exemplary mature form of FGF1 with point mutations R35E, S47V and C117V, wherein numbering refers to SEQ ID NO: 5.

SEQ ID NO: 314 provides an exemplary mature form of FGF1 with point mutations K12V, Y94V, S47V and C117V, wherein numbering refers to SEQ ID NO: 5.

SEQ ID NO: 315 provides an exemplary mature form of FGF1 with point mutation Y15F, wherein numbering refers to SEQ ID NO: 5.

SEQ ID NO: 316 provides an exemplary N-terminally truncated form of FGF1 with point mutation Y15F, wherein numbering refers to SEQ ID NO: 5.

SEQ ID NO: 317 provides an exemplary mature form of FGF1 with point mutations K12V, Y15F and N95V, wherein numbering refers to SEQ ID NO: 5.

SEQ ID NO: 318 provides an exemplary N-terminally truncated form of FGF1 with point mutations K12V, N95V, Y15F and C117V, wherein numbering refers to SEQ ID NO: 5.

SEQ ID NO: 319 provides an exemplary mature form of FGF1 with point mutations Y15F and C117V, wherein numbering refers to SEQ ID NO: 5.

SEQ ID NO: 320 provides an exemplary mature form of FGF1 with point mutations Y15F and A66C, wherein numbering refers to SEQ ID NO: 5.

SEQ ID NO: 321 provides an exemplary mature form of FGF1 with point mutations R35E, Y15F and C117V, wherein numbering refers to SEQ ID NO: 5.

SEQ ID NO: 322 provides an exemplary mature form of FGF1 with point mutations K12V, Y94V, Y15F and C117V, wherein numbering refers to SEQ ID NO: 5.

SEQ ID NO: 323 provides an exemplary mature form of FGF1 with point mutation Y15A, wherein numbering refers to SEQ ID NO: 5.

SEQ ID NO: 324 provides an exemplary N-terminally truncated form of FGF1 with point mutation Y15A, wherein numbering refers to SEQ ID NO: 5.

SEQ ID NO: 325 provides an exemplary mature form of FGF1 with point mutations K12V, Y15A and N95V, wherein numbering refers to SEQ ID NO: 5.

SEQ ID NO: 326 provides an exemplary N-terminally truncated form of FGF1 with point mutations K12V, N95V, Y15A and C117V, wherein numbering refers to SEQ ID NO: 5.

SEQ ID NO: 327 provides an exemplary mature form of FGF1 with point mutations Y15A and C117V, wherein numbering refers to SEQ ID NO: 5.

SEQ ID NO: 328 provides an exemplary mature form of FGF1 with point mutations Y15A and A66C, wherein numbering refers to SEQ ID NO: 5.

SEQ ID NO: 329 provides an exemplary mature form of FGF1 with point mutations R35E, Y15A and C117V, wherein numbering refers to SEQ ID NO: 5.

SEQ ID NO: 330 provides an exemplary mature form of FGF1 with point mutations K12V, Y94V, Y15A and C117V, wherein numbering refers to SEQ ID NO: 5.

SEQ ID NO: 331 provides an exemplary mature form of FGF1 with point mutation L133V, wherein numbering refers to SEQ ID NO: 5.

SEQ ID NO: 332 provides an exemplary N-terminally truncated form of FGF1 with point mutation L133V, wherein numbering refers to SEQ ID NO: 5.

SEQ ID NO: 333 provides an exemplary mature form of FGF1 with point mutations K12V, L133V and N95V, wherein numbering refers to SEQ ID NO: 5.

SEQ ID NO: 334 provides an exemplary N-terminally truncated form of FGF1 with point mutations K12V, N95V, L133V and C117V, wherein numbering refers to SEQ ID NO: 5.

SEQ ID NO: 335 provides an exemplary mature form of FGF1 with point mutations L133V and C117V, wherein numbering refers to SEQ ID NO: 5.

SEQ ID NO: 336 provides an exemplary mature form of FGF1 with point mutations L133V and A66C, wherein numbering refers to SEQ ID NO: 5.

SEQ ID NO: 337 provides an exemplary mature form of FGF1 with point mutations R35E, L133V and C117V, wherein numbering refers to SEQ ID NO: 5.

SEQ ID NO: 338 provides an exemplary mature form of FGF1 with point mutations K12V, Y94V, L133V and C117V, wherein numbering refers to SEQ ID NO: 5.

SEQ ID NO: 339 provides an exemplary mature form of FGF1 with point mutation L133A, wherein numbering refers to SEQ ID NO: 5.

SEQ ID NO: 340 provides an exemplary N-terminally truncated form of FGF1 with point mutation L133A, wherein numbering refers to SEQ ID NO: 5.

SEQ ID NO: 341 provides an exemplary mature form of FGF1 with point mutations K12V, L133A and N95V, wherein numbering refers to SEQ ID NO: 5.

SEQ ID NO: 342 provides an exemplary N-terminally truncated form of FGF1 with point mutations K12V, N95V, L133A and C117V, wherein numbering refers to SEQ ID NO: 5.

SEQ ID NO: 343 provides an exemplary mature form of FGF1 with point mutations L133A and C117V, wherein numbering refers to SEQ ID NO: 5.

SEQ ID NO: 344 provides an exemplary mature form of FGF1 with point mutations L133V and A66C, wherein numbering refers to SEQ ID NO: 5.

SEQ ID NO: 345 provides an exemplary mature form of FGF1 with point mutations R35E, L133A and C117V, wherein numbering refers to SEQ ID NO: 5.

SEQ ID NO: 346 provides an exemplary mature form of FGF1 with point mutations K12V, Y94V, L133A and C117V, wherein numbering refers to SEQ ID NO: 5.

SEQ ID NO: 347 provides an exemplary mature form of FGF1 with point mutation R35K, wherein numbering refers to SEQ ID NO: 5.

SEQ ID NO: 348 provides an exemplary N-terminally truncated form of FGF1 with point mutation R35K, wherein numbering refers to SEQ ID NO: 5.

SEQ ID NO: 349 provides an exemplary mature form of FGF1 with point mutations K12V, R35K and N95V, wherein numbering refers to SEQ ID NO: 5.

SEQ ID NO: 350 provides an exemplary N-terminally truncated form of FGF1 with point mutations K12V, N95V, R35K and C117V, wherein numbering refers to SEQ ID NO: 5.

SEQ ID NO: 351 provides an exemplary mature form of FGF1 with point mutations R35K and C117V, wherein numbering refers to SEQ ID NO: 5.

SEQ ID NO: 352 provides an exemplary mature form of FGF1 with point mutations R35K and A66C, wherein numbering refers to SEQ ID NO: 5.

SEQ ID NO: 353 provides an exemplary mature form of FGF1 with point mutations R35K and C117V, wherein numbering refers to SEQ ID NO: 5.

SEQ ID NO: 354 provides an exemplary mature form of FGF1 with point mutations K12V, Y94V, R35K and C117V, wherein numbering refers to SEQ ID NO: 5.

SEQ ID NO: 355 provides an exemplary mature form of FGF1 with point mutation E87Q, wherein numbering refers to SEQ ID NO: 5.

SEQ ID NO: 356 provides an exemplary N-terminally truncated form of FGF1 with point mutation E87Q, wherein numbering refers to SEQ ID NO: 5.

SEQ ID NO: 357 provides an exemplary mature form of FGF1 with point mutations K12V, E87Q and N95V, wherein numbering refers to SEQ ID NO: 5.

SEQ ID NO: 358 provides an exemplary N-terminally truncated form of FGF1 with point mutations K12V, N95V, E87Q and C117V, wherein numbering refers to SEQ ID NO: 5.

SEQ ID NO: 359 provides an exemplary mature form of FGF1 with point mutations E87Q and C117V, wherein numbering refers to SEQ ID NO: 5.

SEQ ID NO: 360 provides an exemplary mature form of FGF1 with point mutations E87Q and A66C, wherein numbering refers to SEQ ID NO: 5.

SEQ ID NO: 361 provides an exemplary mature form of FGF1 with point mutations R35E, E87Q and C117V, wherein numbering refers to SEQ ID NO: 5.

SEQ ID NO: 362 provides an exemplary mature form of FGF1 with point mutations K12V, Y94V, E87Q and C117V, wherein numbering refers to SEQ ID NO: 5.

SEQ ID NO: 363 provides an exemplary mature form of FGF1 with point mutation E87D, wherein numbering refers to SEQ ID NO: 5.

SEQ ID NO: 364 provides an exemplary N-terminally truncated form of FGF1 with point mutation E87D, wherein numbering refers to SEQ ID NO: 5.

SEQ ID NO: 365 provides an exemplary mature form of FGF1 with point mutations K12V, E87D and N95V, wherein numbering refers to SEQ ID NO: 5.

SEQ ID NO: 366 provides an exemplary N-terminally truncated form of FGF1 with point mutations K12V, N95V, E87D and C117V, wherein numbering refers to SEQ ID NO: 5.

SEQ ID NO: 367 provides an exemplary mature form of FGF1 with point mutations E87D and C117V, wherein numbering refers to SEQ ID NO: 5.

SEQ ID NO: 368 provides an exemplary mature form of FGF1 with point mutations E87D and A66C, wherein numbering refers to SEQ ID NO: 5.

SEQ ID NO: 369 provides an exemplary mature form of FGF1 with point mutations R35E, E87D and C117V, wherein numbering refers to SEQ ID NO: 5.

SEQ ID NO: 370 provides an exemplary mature form of FGF1 with point mutations K12V, Y94V, E87D and C117V, wherein numbering refers to SEQ ID NO: 5.

SEQ ID NO: 371 provides an exemplary mature form of FGF1 with point mutation Y8F, wherein numbering refers to SEQ ID NO: 5.

SEQ ID NO: 372 provides an exemplary mature form of FGF1 with point mutations K12V, Y8F and N95V, wherein numbering refers to SEQ ID NO: 5.

SEQ ID NO: 373 provides an exemplary mature form of FGF1 with point mutations Y8F and C117V, wherein numbering refers to SEQ ID NO: 5.

SEQ ID NO: 374 provides an exemplary mature form of FGF1 with point mutations Y8F and A66C, wherein numbering refers to SEQ ID NO: 5.

SEQ ID NO: 375 provides an exemplary mature form of FGF1 with point mutations R35E, Y8F and C117V, wherein numbering refers to SEQ ID NO: 5.

SEQ ID NO: 376 provides an exemplary mature form of FGF1 with point mutations K12V, Y94V, Y8F and C117V, wherein numbering refers to SEQ ID NO: 5.

SEQ ID NO: 377 provides an exemplary mature form of FGF1 with point mutation Y8V, wherein numbering refers to SEQ ID NO: 5.

SEQ ID NO: 378 provides an exemplary mature form of FGF1 with point mutations K12V, Y8V and N95V, wherein numbering refers to SEQ ID NO: 5.

SEQ ID NO: 379 provides an exemplary mature form of FGF1 with point mutations Y8V and C117V, wherein numbering refers to SEQ ID NO: 5.

SEQ ID NO: 380 provides an exemplary mature form of FGF1 with point mutations Y8V and A66C, wherein numbering refers to SEQ ID NO: 5.

SEQ ID NO: 381 provides an exemplary mature form of FGF1 with point mutations R35E, Y8V and C117V, wherein numbering refers to SEQ ID NO: 5.

SEQ ID NO: 382 provides an exemplary mature form of FGF1 with point mutations K12V, Y94V, Y8V and C117V, wherein numbering refers to SEQ ID NO: 5.

SEQ ID NO: 383 provides an exemplary mature form of FGF1 with point mutation Y8A, wherein numbering refers to SEQ ID NO: 5.

SEQ ID NO: 384 provides an exemplary mature form of FGF1 with point mutations K12V, Y8A and N95V, wherein numbering refers to SEQ ID NO: 5.

SEQ ID NO: 385 provides an exemplary mature form of FGF1 with point mutations Y8A and C117V, wherein numbering refers to SEQ ID NO: 5.

SEQ ID NO: 386 provides an exemplary mature form of FGF1 with point mutations Y8A and A66C, wherein numbering refers to SEQ ID NO: 5.

SEQ ID NO: 387 provides an exemplary mature form of FGF1 with point mutations R35E, Y8A and C117V, wherein numbering refers to SEQ ID NO: 5.

SEQ ID NO: 388 provides an exemplary mature form of FGF1 with point mutations K12V, Y94V, Y8A and C117V, wherein numbering refers to SEQ ID NO: 5.

SEQ ID NO: 389 provides an exemplary mature form of FGF1 with point mutation K9R, wherein numbering refers to SEQ ID NO: 5.

SEQ ID NO: 390 provides an exemplary mature form of FGF1 with point mutations K12V, K9R and N95V, wherein numbering refers to SEQ ID NO: 5.

SEQ ID NO: 391 provides an exemplary mature form of FGF1 with point mutations K9R and C117V, wherein numbering refers to SEQ ID NO: 5.

SEQ ID NO: 392 provides an exemplary mature form of FGF1 with point mutations K9R and A66C, wherein numbering refers to SEQ ID NO: 5.

SEQ ID NO: 393 provides an exemplary mature form of FGF1 with point mutations R35E, K9R and C117V, wherein numbering refers to SEQ ID NO: 5.

SEQ ID NO: 394 provides an exemplary mature form of FGF1 with point mutations K12V, Y94V, K9R and C117V, wherein numbering refers to SEQ ID NO: 5.

SEQ ID NO: 395 provides an exemplary mature form of FGF1 with point mutation K9A, wherein numbering refers to SEQ ID NO: 5.

SEQ ID NO: 396 provides an exemplary mature form of FGF1 with point mutations K12V, K9A and N95V, wherein numbering refers to SEQ ID NO: 5.

SEQ ID NO: 397 provides an exemplary mature form of FGF1 with point mutations K9A and C117V, wherein numbering refers to SEQ ID NO: 5.

SEQ ID NO: 398 provides an exemplary mature form of FGF1 with point mutations K9A and A66C, wherein numbering refers to SEQ ID NO: 5.

SEQ ID NO: 399 provides an exemplary mature form of FGF1 with point mutations R35E, K9A and C117V, wherein numbering refers to SEQ ID NO: 5.

SEQ ID NO: 400 provides an exemplary mature form of FGF1 with point mutations K12V, Y94V, K9A and C117V, wherein numbering refers to SEQ ID NO: 5.

SEQ ID NO: 401 (Salk_073) provides an exemplary N-terminally truncated form of FGF1 with point mutations K12V, A66C, N95V, S116R, and C117V, wherein numbering refers to SEQ ID NO: 5.

SEQ ID NO: 402 (Salk_074) provides an exemplary N-terminally truncated form of FGF1 with point mutations K12V, H21Y, L44F, A66C, N95V, H102Y, F108Y, and C117V, wherein numbering refers to SEQ ID NO: 5.

SEQ ID NO: 403 provides an exemplary mature form of FGF1 with a point mutation S116R, wherein numbering refers to SEQ ID NO: 5, designed to increase affinity for heparan sulfate.

SEQ ID NO: 404 provides an exemplary mature form of FGF1 with point mutations K12V, N95V, S116R, C117V, wherein numbering refers to SEQ ID NO: 5.

SEQ ID NO: 405 provides an exemplary N-terminally truncated form of FGF1 with point mutations S116R and C117V, wherein numbering refers to SEQ ID NO: 5.

SEQ ID NO: 406 provides an exemplary N-terminally truncated form of FGF1 with point mutations N95V, S116R and C117V, wherein numbering refers to SEQ ID NO: 5.

SEQ ID NO: 407 provides an exemplary mature form of FGF1 with point mutations K12V, N95T, S116R, and C117V, wherein numbering refers to SEQ ID NO: 5.

SEQ ID NO: 408 provides an exemplary mature form of FGF1 with point mutations Y55A, S116R, and C117V, wherein numbering refers to SEQ ID NO: 5.

SEQ ID NO: 409 provides an exemplary mature form of FGF1 with point mutations Y55W, S116R, and C117Vwherein numbering refers to SEQ ID NO: 5.

SEQ ID NO: 410 provides an exemplary mature form of FGF1 with point mutations E87H, S116R, and C117V, wherein numbering refers to SEQ ID NO: 5.

SEQ ID NO: 411 provides an exemplary N-terminally truncated form of FGF1 with point mutations R35E, S116R, and C117V, wherein numbering refers to SEQ ID NO: 5.

SEQ ID NO: 412 provides an exemplary N-terminally truncated form of FGF1 with point mutations C117V, wherein numbering refers to SEQ ID NO: 5.

SEQ ID NO: 413 provides an exemplary mature form of FGF1 with point mutations E49A, S116R, and C117V, wherein numbering refers to SEQ ID NO: 5.

SEQ ID NO: 414 provides an exemplary N-terminally truncated form of FGF1 with point mutations K12V, Y94V, N95V, S116R, and C117V, wherein numbering refers to SEQ ID NO: 5.

SEQ ID NO: 415 provides an exemplary N-terminally truncated form of FGF1 with point mutations K12V, N95V, S116R, C117V, underlined, wherein numbering refers to SEQ ID NO: 5.

SEQ ID NO: 416 provides an exemplary mature form of FGF1 with point mutation H93G, wherein numbering refers to SEQ ID NO: 5.

SEQ ID NO: 417 provides an exemplary mature form of FGF1 with point mutations Q40P, S47I, and H93G, wherein numbering refers to SEQ ID NO: 5.

SEQ ID NO: 418 provides an exemplary mature form of FGF1 with point mutation K12V, wherein numbering refers to SEQ ID NO: 5.

SEQ ID NO: 419 provides an exemplary mature form of FGF1 with point mutations K12V and N95V, wherein numbering refers to SEQ ID NO: 5.

SEQ ID NO: 420 provides an exemplary N-terminally truncated form of FGF1 with point mutations K12V, H21Y, L44F, N95V, H102Y, F108Y, and C117V, wherein numbering refers to SEQ ID NO: 5,and wherein the removed amino acids from the N-terminus are replaced with a peptide to target FGF1Rβ (SYNHLQGDVR; amino acids 1 to 10 of SEQ ID NO: 420).

SEQ ID NO: 421 provides an exemplary N-terminally truncated form of FGF1 with point mutations K12V, H21Y, L44F, N95V, H102Y, F108Y, and C117V, wherein numbering refers to SEQ ID NO: 5,and wherein the removed amino acids from the N-terminus are replaced with a peptide to target FGF1Rβ (SYNHLQGDVRV; amino acids 1 to 11 of SEQ ID NO: 421).

SEQ ID NO: 422 provides an exemplary N-terminally truncated form of FGF1 with point mutations K12V, H21Y, L44F, N95V, H102Y, F108Y, and C117V, wherein numbering refers to SEQ ID NO: 5,and wherein the removed amino acids from the N-terminus are replaced with a peptide to target FGF1β (SYDYMEGGDIRV; amino acids 1 to 11 of SEQ ID NO: 422).

SEQ ID NO: 423 provides an exemplary sequence for targeting to the vagus nerve, referred to as exendin-4.

SEQ ID NO: 424 provides an exemplary chimeric sequence comprising an N-terminal vagus nerve targeting sequence (SEQ ID NO: 423), a linker (amino acids 40 to 45), and a mature FGF1 sequence (SEQ ID NO: 5).

SEQ ID NO: 425 (Salk-082) provides an exemplary chimeric sequence comprising an N-terminal vagus nerve targeting sequence (SEQ ID NO: 423), a linker (amino acids 40 to 45), and a mutant FGF1 sequence comprising a C117V mutation (SEQ ID NO: 138).

SEQ ID NO: 426 (Salk-087) provides an exemplary chimeric sequence comprising an N-terminal vagus nerve targeting sequence that is an N-terminally truncated version of exendin 4 (SEQ ID NO: 434), a linker (amino acids 32 to 37), and a mutant FGF1 sequence comprising a C117V mutation (SEQ ID NO: 138).

SEQ ID NO: 427 (Salk-088) provides an exemplary chimeric sequence comprising a C-terminal vagus nerve targeting sequence (exendin 4, SEQ ID NO: 423), a linker (amino acids 141 to 146), and a mutant FGF1 sequence comprising a C117V mutation (SEQ ID NO: 138).

SEQ ID NO: 428 (Salk-089) provides an exemplary chimeric sequence comprising an N-terminal vagus nerve targeting sequence (exendin 4, SEQ ID NO: 423), a linker (amino acids 40 to 45), and a mutant FGF1 sequence comprising mutations K12V, N95V, and C117V (SEQ ID NO: 67).

SEQ ID NO: 429 (Salk-090) provides an exemplary chimeric sequence comprising an N-terminal vagus nerve targeting sequence (exendin 4, SEQ ID NO: 423), a linker (amino acids 40 to 45), and a mutant FGF1 sequence comprising mutations K12V, H21Y, L44F, N95V, H102Y, F108Y, and C117V (SEQ ID NO: 67).

SEQ ID NO: 430 (Salk-091) provides an exemplary chimeric sequence comprising an N-terminal vagus nerve targeting sequence (exendin 4, SEQ ID NO: 423), a linker (amino acids 40 to 45), and a mutant N-terminally truncated FGF1 sequence comprising mutations K12V, H21Y, L44F, N95V, H102Y, F108Y, and C117V (SEQ ID NO: 14). This mutant FGF1 has reduced mitogenicity.

SEQ ID NO: 431 (Salk-092) provides an exemplary chimeric sequence comprising an N-terminal vagus nerve targeting sequence (exendin 4, SEQ ID NO: 423), a linker (amino acids 40 to 45), and a mutant N-terminally truncated FGF1 sequence comprising mutations K12V, H21Y, L44F, N95V, H102Y, F108Y, and C117V (SEQ ID NO: 421).

SEQ ID NO: 432 (Salk-093) provides an exemplary chimeric sequence comprising an N-terminal vagus nerve targeting sequence (oxyntomodulin, SEQ ID NO: 435), a linker (amino acids 38 to 43), and a mutant FGF1 sequence comprising a C117V mutation (SEQ ID NO: 138).

SEQ ID NO: 433 (Salk-094) provides an exemplary chimeric sequence comprising an N-terminal vagus nerve targeting sequence (PYY, SEQ ID NO: 436), a linker (amino acids 35 to 40), and a mutant FGF1 sequence comprising a C117V mutation (SEQ ID NO: 138).

SEQ ID NO: 434 is an N-terminally truncated version of exendin 4 (amino acids 9-39 of SEQ ID NO: 423) that can be used to target a chimeric protein to the vagus nerve.

SEQ ID NO: 435 is an exemplary peptide, oxyntomodulin, which can be used to target a chimeric protein to the vagus nerve. This peptide contains the 29 amino acid sequence of glucagon followed by an 8 amino acid C-terminal extension. This peptide mimics the effects of GLP1.

SEQ ID NO: 436 is an exemplary peptide, PYY, which can be used to target a chimeric protein to the vagus nerve.

SEQ ID NOS: 437-438 are exemplary vagus nerve targeting peptides.

SEQ ID NOS: 439 and 440 are exemplary linker sequences.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Dec. 15, 2016. All references, including patents and patent applications, and GenBank® Accession numbers cited herein are incorporated by reference in their entireties.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as a mutated FGF1 protein, FGF1-vagus targeting chimeric protein, or nucleic acid molecule encoding such, by any effective route. Exemplary routes of administration include, but are not limited to, oral, injection (such as subcutaneous, intraosseous, intramuscular, intradermal, intraperitoneal, intravenous, intrathecal, and intratumoral), sublingual, rectal, transdermal, intranasal, vaginal and inhalation routes. C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Chimeric protein: A protein that includes at least a portion of the sequence of a first protein (e.g., FGF1, such as a mutant FGF1) and at least a portion of the sequence of a full-length second protein (e.g., a protein that targets the vagus nerve), where the first and second proteins are different. A chimeric polypeptide also encompasses polypeptides that include two or more non-contiguous portions derived from the same polypeptide. The two different peptides can be joined directly or indirectly, for example using a linker.

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes results from insulin resistance, a condition in which cells fail to use insulin properly, sometimes combined with an absolute insulin deficiency. This form is also called “non-insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level≥7.0 mmol/l (126 mg/dl);
b. Plasma glucose≥11.1 mmol/l (200 mg/dL) two hours after a 75 g oral glucose load as in a glucose tolerance test;
c. Symptoms of hyperglycemia and casual plasma glucose≥11.1 mmol/l (200 mg/dl);
d. Glycated hemoglobin (Hb A1C)≥6.5%

Effective amount or therapeutically effective amount: The amount of agent, such as a mutated FGF1 protein and/or FGF1-vagus targeting chimeric protein (or nucleic acid molecules encoding such) disclosed herein, that is an amount sufficient to prevent, treat (including prophylaxis), reduce, and/or ameliorate the symptoms and/or underlying causes of any of a disorder or disease. In one embodiment, an “effective amount” is sufficient to reduce or eliminate a symptom of a disease, such as a diabetes (such as type II diabetes), for example by lowering blood glucose.

Fibroblast Growth Factor 1 (FGF1): e.g., OMIM 13220. Includes FGF1 nucleic acid molecules and proteins. FGF1 is a protein that binds to the FGF receptor and is also known as the acidic FGF. FGF1 sequences are publically available, for example from GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). One of ordinary skill in the art can identify additional FGF1 nucleic acid and protein sequences, including FGF1 variants.

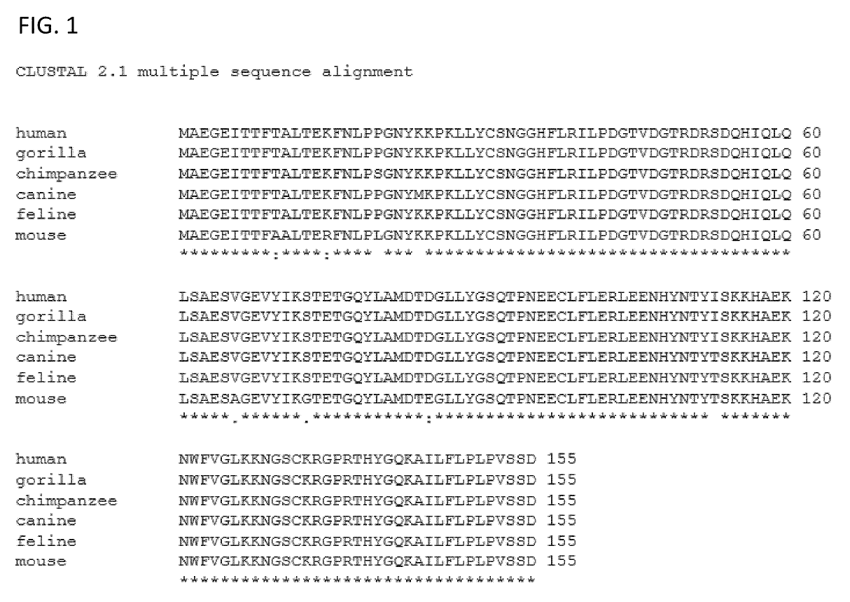

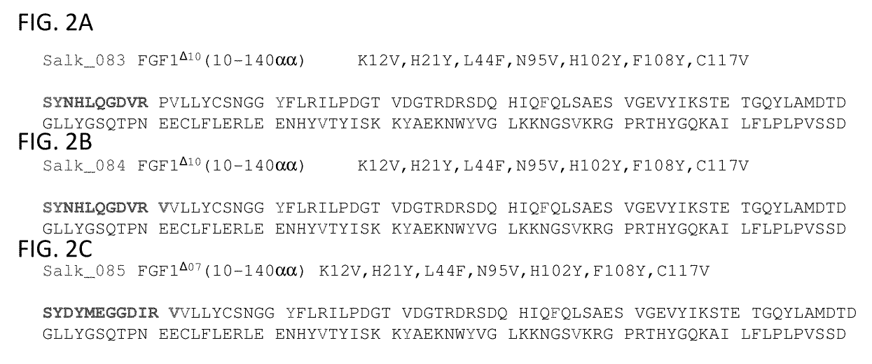

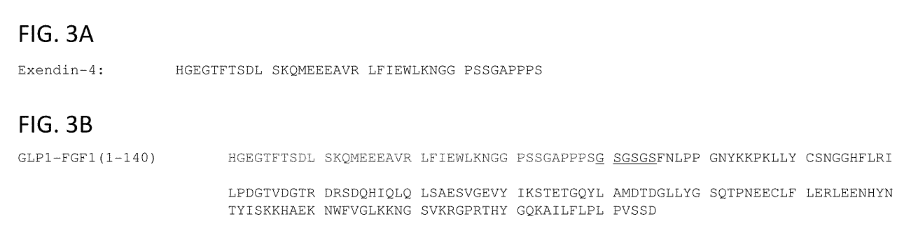

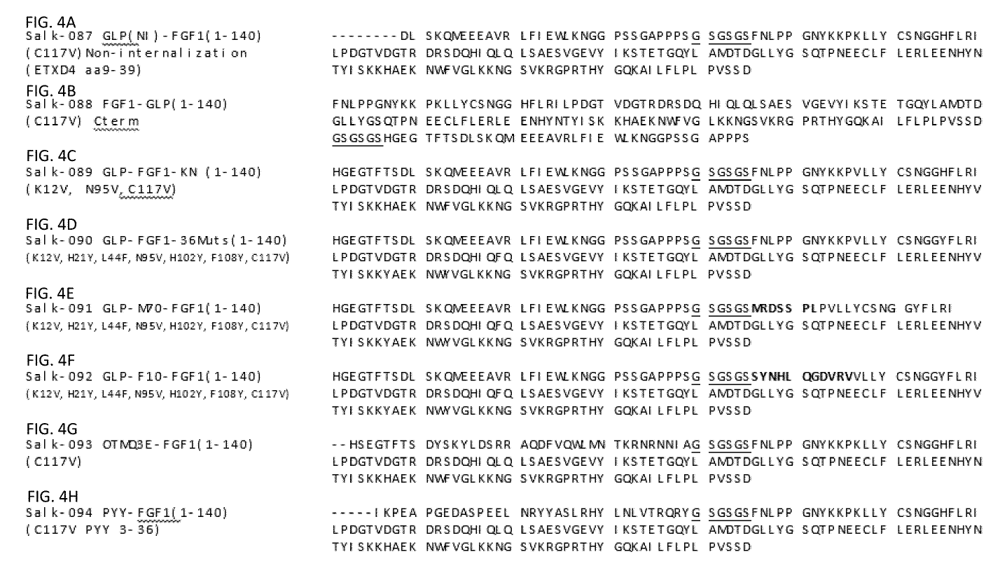

Specific examples of native FGF1 sequences are provided in SEQ ID NOS: 1-9 and shown in FIG. 1. A native FGF1 sequence is one that does not include a mutation that alters the normal activity of the protein (e.g., activity of SEQ ID NOS: 2, 4 or 5-9). A mature FGF1 refers to an FGF1 peptide or protein product and/or sequence following any post-translational modifications. A mutated FGF1 is a variant of FGF1 with different or altered biological activity, such as reduced mitogenicity (e.g., a variant of any of SEQ ID NOS: 1-9, such as one having at least 90%, at least 95%, at least 96%, at least 97%, at least 98% or at least 99% sequence identity to any of SEQ ID NOS: 10-422, but is not a native/wild-type sequence, and in some examples retains the point mutation(s) noted herein for that sequence). In one example, such a variant includes an N-terminal truncation and/or one or more additional point muatations (such as one or more of those shown in Table 1), such as changes that decrease mitogenicity of FGF1, alter the heparin binding affinity of FGF1, and/or the thermostability of FGF1. Specific exemplary FGF1 mutant proteins are shown in SEQ ID NOS: 10-422.

Glucagon-like peptide 1 (GLP1): e.g., OMIM 138030. A hormone that is involved in the normalization of glucose levels in blood. The biologically active forms of GLP1 are: GLP-1-(7-37) (HAEGTFTSDVSSYLEGQAAKEFIAWLVKGRG; SEQ

ID NO: 437) and GLP-1-(7-36) (HAEGTFTSDVSSYLEGQAAKEFIAWLVKGR-NH2; SEQ ID NO: 438), which result from selective cleavage of the proglucagon molecule. Includes GLP1 nucleic acid molecules and proteins. GLP1 sequences are publically available, for example from GenBank® sequence database. One of ordinary skill in the art can identify additional GLP1 nucleic acid and protein sequences, including GLP1 variants of SEQ ID NO: 437 and 438, such as variants that retain the ability to bind GLP1R and regulate blood glucose. GLP1, as well as GLP1 agonists or mimetics (such as those that bind and activate the GLP1R) can be conjugated directly or indirectly (e.g., used to target a protein to the vagus nerve), such as dulaglutide, liraglutide, lixisenatide, albiglutide, or combinations thereof.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a mutant FGF1 protein or a FGF1-vagus targeting chimeric protein disclosed herein.

Isolated: An “isolated” biological component (such as a mutated FGF1 protein, FGF1-vagus targeting chimeric protein, or nucleic acid molecule encoding such) has been substantially separated, produced apart from, or purified away from other biological components in the cell of the organism in which the component naturally occurs, such as other chromosomal and extrachromosomal DNA and RNA, and proteins. Nucleic acid molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, protein, or nucleic acid molecule can be at least 70%, at least 80%, at least 90%, at least 95%, at least 96%, at least 97%, at least 98%, or at least 99% pure.

Linker: A moiety or group of moieties that joins or connects two or more discrete separate peptide or proteins, such as monomer domains, for example to generate a chimeric protein. In one example a linker is a substantially linear moiety. Exemplary linkers that can be used to generate the chimeric proteins provided herein include but are not limited to: peptides, nucleic acid molecules, peptide nucleic acids, and optionally substituted alkylene moieties that have one or more oxygen atoms incorporated in the carbon backbone. A linker can be a portion of a native sequence, a variant thereof, or a synthetic sequence. Linkers can include naturally occurring amino acids, non-naturally occurring amino acids, or a combination of both. In one example a linker is composed of at least 5, at least 10, at least 15 or at least 20 amino acids, such as 5 to 10, 5 to 20, or 5 to 50 amino acids. In one example the linker is a polyalanine. In one example the linker is a flexible linker, such as one that includes Gly and Ser residues (e.g., GSGSGS (SEQ ID NO: 439) or GGSGGGGSGG, SEQ ID NO: 440).

Mammal: This term includes both human and non-human mammals. Similarly, the term “subject” includes both human and veterinary subjects (such as cats, dogs, cows, and pigs) and rodents (such as mice and rats).

Metabolic disorder/disease: A disease or disorder that results from the disruption of the normal mammalian process of metabolism. For example, a metabolic disorder/disease includes metabolic syndrome.

Other examples include, but are not limited to, (1) glucose utilization disorders and the sequelae associated therewith, including diabetes mellitus (Type 1 and Type 2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thrombosis, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinflammatory processes and/or other peripheral neuropathies, including Alzheimer's disease, multiple sclerosis, Parkinson's disease, progressive multifocal leukoencephalopathy, and Guillain-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

N-terminal portion: A region of a protein sequence that includes a contiguous stretch of amino acids that begins at or near the N-terminal residue of the protein. An N-terminal portion of the protein can be defined by a contiguous stretch of amino acids (e.g., a number of amino acid residues).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a mutated FGF1 coding sequence or a FGF1-vagus targeting chimeric protein coding sequence). In one example, an FGF1 coding sequence is operably linked to a vagus targeting protein coding sequence, to generate an FGF1-vagus targeting chimeric protein. Generally, operably linked DNA sequences are contiguous and, where necessary, join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15^{th} Edition (1975), describes compositions and formulations suitable for pharmaceutical delivery of the disclosed mutated FGF1 proteins and/or FGF1-vagus targeting chimeric proteins (or nucleic acid molecules encoding such) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid molecule is one that has a sequence that is not naturally occurring (e.g., a mutated FGF1 protein or an FGF1-vagus targeting chimeric protein) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant or transgenic cell is one that contains a recombinant nucleic acid molecule and expresses a recombinant protein.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn, and tblastx. A description of how to determine sequence identity using this program is available on the NCBI website on the internet.

Variants of the mutated FGF1 proteins and coding sequences disclosed herein, as well as the FGF1-vagus targeting chimeric proteins and coding sequences disclosed herein, are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a mutant FGF1 protein provided herein, can share at least 80%, at least 85%, at least 90%, at least 91%, at least 92%, at least 95%, at least 96%, at least 97%, at least 98% or at least 99% sequence identity to any one of SEQ ID NOS: 10-422 (such as to SEQ ID NO: 420, 421 or 422), but is not SEQ ID NOS: 2, 4, or 5 (which, in some examples, has the point mutation(s) recited herein for that sequence, such as one or more, such as 1, 2, 3, 4, 5, 6, 7, 8, 9, or 10 of the mutations shown in Table 1). In addition, exemplary mutated FGF1 proteins have at least 80%, at least 85%, at least 90%, at least 92%, at least 95%, at least 96%, at least 97%, at least 98% or at least 99% sequence identity to any one of SEQ ID NOS: 10-422 (such as to SEQ ID NO: 420, 421 or 422), and retain the ability to reduce blood glucose levels in vivo.

Similarly, an FGF1-vagus targeting chimeric protein provided herein, can share at least 80%, at least 85%, at least 90%, at least 91%, at least 92%, at least 95%, at least 96%, at least 97%, at least 98% or at least 99% sequence identity to any one of SEQ ID NOS: 424, 425, 426, 427, 428, 429, 430, 431, 432, and 433 (which, in some examples, the FGF1 portion has the point mutation(s) recited herein for that sequence, such as one or more, such as 1, 2, 3, 4, 5, 6, 7, 8, 9, or 10 of the mutations shown in Table 1). In addition, exemplary FGF1-vagus targeting chimeric proteins have at least 80%, at least 85%, at least 90%, at least 92%, at least 95%, at least 96%, at least 97%, at least 98% or at least 99% sequence identity to any one of SEQ ID NOS: 424, 425, 426, 427, 428, 429, 430, 431, 432, and 433, and retain the ability to reduce blood glucose levels in vivo.

Subject: Any mammal, such as humans, non-human primates, pigs, sheep, cows, dogs, cats, rodents and the like which is to be the recipient of the particular treatment, such as treatment with a mutated FGF1 protein and/or an FGF1-vagus targeting chimeric protein (or corresponding nucleic acid molecule) provided herein. In two non-limiting examples, a subject is a human subject or a murine subject. In some examples, the subject has one or more metabolic diseases, such as diabetes (e.g., type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular disease (e.g., hypertension), or combinations thereof. In some examples, the subject has elevated blood glucose.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed., Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Transgene: An exogenous gene supplied by a vector. In one example, a transgene includes a mutated FGF1 coding sequence. In one example, a transgene includes a FGF1-vagus targeting chimeric protein coding sequence.

Vector: A nucleic acid molecule as introduced into a host cell, thereby producing a transformed host cell. A vector may include nucleic acid sequences that permit it to replicate in the host cell, such as an origin of replication. A vector may also include one or more mutated FGF1 coding sequences, one or more FGF1-vagus targeting chimera coding sequences, and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like.

### Overview

Provided herein are mutated FGF1 proteins, which can include an N-terminal deletion, one or more additional point mutations (such as amino acid substitutions, deletions, additions, or combinations thereof), or combinations of an N-terminal deletion and an additional one or more point mutations. Also provided are chimeric proteins, which include an FGF1 protein (such as a native or mutated FGF1 protein), and a peptide that targets the chimeric protein to the vagus nerve. Such chimeric proteins are referred to herein as FGF1-vagus targeting chimeric proteins.

Also provided are methods of using the disclosed FGF1 mutant proteins and FGF1-vagus targeting chimeric proteins (or their nucleic acid coding sequences) to lower glucose, for example to treat one or more metabolic diseases, or combinations thereof. Exemplary metabolic diseases that can be treated with the disclosed methods include, but are not limited to: type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular diseases (e.g., hypertension), latent autoimmune diabetes (LAD), or maturity onset diabetes of the young (MODY).

In some examples, an FGF1 mutant protein has at least 80%, at least 85%, at least 90%, at least 92%, at least 95%, at least 96%, at least 97%, at least 98%, at least 99%, or 100% sequence identity to SEQ ID NO: 420, 421, or 422. In some examples, an FGF1 mutant protein having at least 80%, at least 85%, at least 90%, at least 92%, at least 95%, at least 96%, at least 97%, at least 98%, at least 99% sequence identity to

SEQ ID NO: 420, 421, or 422 retains the point mutation(s) described herein for that sequence. For example, an FGF1 mutant protein having at least 80%, at least 85%, at least 90%, at least 92%, at least 95%, at least 96%, at least 97%, at least 98%, at least 99% sequence identity to SEQ ID NO: 420, 421, or 422 can retain the K12V, H21Y, L44F, N95V, H102Y, F108Y, and C117V mutations. In some examples, the FGF1 mutant protein includes or consists of any of one SEQ ID NOS: 420, 421, or 422. The disclosure encompasses variants of the disclosed FGF1 mutant proteins, such as any of one SEQ ID NOS: 420, 421, or 422 having 1 to 8, 2 to 10, 1 to 5, 1 to 6, or 5 to 10 additional mutations, such as conservative amino acid substitutions.

FGF1-vagus targeting chimeric proteins include at least two portions, an FGF1 protein and a vagus nerve targeting protein. The FGF1 protein portion of the FGF1-vagus targeting chimeric protein can be a native FGF1 protein (such as SEQ ID NO: 2 or 5), or a mutated FGF1 protein (such a protein having at least 80%, at least 85%, at least 90%, at least 95%, at least 96%, at least 97%, at least 99%, or 100% sequence identity to any one of SEQ ID NOS: 10-422). In some examples, the FGF1 mutant protein (of the FGF1-vagus targeting chimeric protein) having at least 80%, at least 85%, at least 90%, at least 92%, at least 95%, at least 96%, at least 97%, at least 98%, at least 99% sequence identity to any of SEQ ID NOS: 10-422 retains the point mutation(s) described herein for that sequence. The vagus nerve targeting protein portion of the FGF1-vagus targeting chimeric protein includes a protein that permits the chimera to target the vagus nerve. Examples of such proteins include GLP1 (e.g., SEQ ID NO: 437 or 438), exendin 4 (e.g., SEQ ID NO: 423) or a truncated version thereof (e.g., SEQ ID NO: 434), oxyntomodulin (e.g., SEQ ID NO: 425), peptide YY (e.g., SEQ ID NO: 426), or variant thereof, such as one having at least 80%, at least 85%, at least 90%, at least 95%, at least 96%, at least 97%, at least 99%, or 100% sequence identity to SEQ ID NO: 423, 434, 435, 436, 437, or 438, and still retain the ability to target the chimeric protein to the vagus nerve. In some examples, the FGF1 protein of the FGF1-vagus targeting chimeric protein is directly attached to the vagus nerve targeting protein, such as at either the N-terminus or the C-terminus. In some examples, the FGF1-vagus targeting chimeric protein comprises a linker between the FGF1 protein and the vagus nerve targeting protein. In specific examples, the FGF1-vagus targeting chimeric protein comprises at least 85%, at least 90%, at least 95%, at least 96%, at least 97%, at least 99%, or 100% sequence identity to SEQ ID NO: 424, 425, 426, 427, 428, 429, 430, 431, 432, or 433.

In some examples, the mutant FGF1 protein, or the FGF1 portion of an FGF1-vagus targeting chimera protein, includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 amino acid substitutions, such as 1-20, 1-10, 4-8, 5-25, 1-5, 1-6, 1-7, 1-8, 2-5, 2-7, 1, 2, 3, 4, 5, 6, 7, 8, 9, 10, 11, 12, 13, 14, 15, 16, 17, 18, 19, 20, 21, 22, 23, 24, or 25 amino acid substitutions (such as those shown in Table 1). In some examples, the mutant FGF1 protein (or the FGF1 portion of an FGF1-vagus targeting chimera protein) further includes deletion of one or more amino acids, such as deletion of 1-10, 4-8, 5-10, 1, 2, 3, 4, 5, 6, 7, 8, 9, 10, 11, 12, 13, 14, 15, 16, 17, 18, 19, or 20 amino acid deletions. In some examples, the mutant

FGF1 protein (or the FGF1 portion of an FGF1-vagus targeting chimera protein) includes a combination of amino acid substitutions and deletions, such as at least 1 substitution and at least 1 deletion, such as 1 to 10 substitutions with 1 to 10 deletions.

TABLE 1
Exemplary FGF1 mutations

| Location of Point | | Location of Point |
|---|---|---|
| Mutation Position | | Mutation Position |
| in SEQ ID NO: 2 | Mutation Citation | in SEQ ID NO: 5 |
| Y23 | Y8F, Y8V, Y8A | Y8 |
| K24 | K9T, K9R, K9A, | K9 |
| K25 | K10T | K10 |
| K27 | K12V | K12 |
| L29 | L14A | L14 |
| Y30 | Y15F, Y15A, Y15V | Y15 |
| C31 | C16V, C16A, C16T, C16S | C16 |
| S32 | S17R, S17K | S17 |
| N33 | N18R, N18K | N18 |
| H36 | H21Y | H21 |
| R50 | R35E, R35V, R35K | R35 |
| Q55 | Q40P | Q40 |
| Q58 | Q43K, Q43E, Q43A | Q43 |
| L59 | L44F | L44 |
| L61 | L46V | L46 |
| S62 | S47I, S47A, S47V, | S47 |
| E64 | E49D, E49K, E49Q, E49A | E49 |
| Y70 | Y55F, Y55V, Y55S, Y55A, | Y55 |
| | Y55W | |
| A81 | A66C | A66 |
| M82 | M67I | M67 |
| L88 | L73V | L73 |
| C98 | C83T, C83S, C83A C83V | C83 |
| E102 | E87V, E87A, E87S, E87T, | E87 |
| | E87Q, E87D, E87H | |
| R103 | R88Y, R88L, R88D | R88 |
| H108 | H93G, H93A | H93 |
| Y109 | Y94V, Y94F, Y94A | Y94 |
| N110 | N95V, N95A, N95S, N95T | N95 |
| S114 | S99A | S99 |
| K116 | K101E | K101 |
| H117 | H102Y, H102A | H102 |
| A118 | A103G | A103 |
| EKN 119-121 | Δ104-106 | EKN |
| | | (104-106) |
| W122 | W107A | W107 |
| F123 | F108Y | F108 |
| V124 | V109L | V109 |
| L126 | L111I | L111 |
| K127 | K112D, K112E, K112Q | K112 |
| K128 | K113Q, K113E, K113D | K113 |
| N129 | N114K, N114R | N114 |
| S131 | S116R | S116 |
| C132 | C117V, C117P, C117T, | C117 |
| | C117S, C117A | |
| K133 | K118N, K118E, K118V | K118 |
| R134 | R119G, R119V, R119E | R119 |
| GPR 135-137 | Δ120-122 | GPR |
| | | (120-122) |
| Q142 | Q127R, Q127K | Q127 |
| F147 | F132W | F132 |
| L148 | L133A, L133V, L133S | L133 |
| P149 | P134V | P134 |
| L150 | L135A, L135S | L135 |

Exemplary mutations that can be made to a mutant FGF1 protein (or to the FGF1 portion of an FGF1-vagus targeting chimera protein) are shown in Table 1, with amino acids referenced to either SEQ ID NOS: 2 or 5. One skilled in the art will recognize that these mutations can be used singly, or in any combination (such as 1-54, 1-10, 1-5, 1-6, 1-7, 1-8, 2-5, 4-8, 2-7, 5-25, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or 54 of these amino acid substitutions and/or deletions).

In some examples, the mutant FGF1 protein (or to the FGF1 portion of an FGF1-vagus targeting chimera protein) includes mutations at one or more of the following positions: Y8, K9, K10, K12, L14, Y15, C16, S17, N18, H21, R35, Q40, Q43, L44, L46, S47, E49, Y55, A66, M67, L73, C83, E87, R88, H93, Y94, N95, S99, K101, H102, A103, E104, K105, N106, W107, F108, V109, L111, K112, K113, N114, S116, C117, K118, R119, G120, P121, R122, Q127, F132, L133, P134, or L135 such as 1 to 3, 1 to 5, 1 to 6, 1 to 7, 1 to 8, 2 to 5, 3 to 5, 3 to 6, 3 to 8, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or 54 of these positions (wherein the position refers to SEQ ID NO: 5).

In some examples, the mutant FGF1 protein (or to the FGF1 portion of an FGF1-vagus targeting chimera protein) includes at least 90 consecutive amino acids from amino acids 5-141 of FGF1 (e.g., of SEQ ID NOS: 2 or 4), (which in some examples can include further deletion of N-terminal amino acids 1-20 and/or point mutations, such as substitutions, deletions, and/or additions). In some examples, the mutant FGF1 protein (or to the FGF1 portion of an FGF1-vagus targeting chimera protein) includes at least 100 or at least 110 consecutive amino acids from amino acids 5-141 of FGF1, such as at least 100 consecutive amino acids from amino acids 5-141 of SEQ ID NO: 2 or 4 or at least 100 consecutive amino acids from SEQ ID NO: 5.

In some examples, the mutant FGF1 (or to the FGF1 portion of an FGF1-vagus targeting chimera protein) protein includes both an N-terminal truncation and additional point mutations. Specific exemplary FGF1 mutant proteins (which can be used as the FGF1 portion of an FGF1-vagus targeting chimera protein) are shown in SEQ ID NOS: 10-422. In some examples, the FGF1 mutant includes an N-terminal deletion, but retains a methionine at the N-terminal position. In some examples, the FGF1 mutant (or to the FGF1 portion of an FGF1-vagus targeting chimera protein) is 120-140 or 125-140 amino acids in length.

Also provided are isolated nucleic acid molecules encoding the disclosed mutated FGF1 proteins and FGF1-vagus targeting chimera proteins, such as a nucleic acid molecule encoding a protein having at least 80%, at least 85%, at least 90%, at least 95%, at least 98%, or at least 99% sequence identity to any of one SEQ ID NOS: 10-422, 424, 425, 426, 427, 428, 429, 430, 431, 432, or 433. Vectors and cells that include such nucleic acid molecules are also provided. For example, such nucleic acid molecules can be expressed in a host cell, such as a bacterium or yeast cell (e.g., E. coli), thereby permitting expression of the mutated FGF1 protein or FGF1-vagus targeting chimera protein. The resulting protein can be purified from the cell.

Methods of using the disclosed mutated FGF1 proteins and FGF1-vagus targeting chimera proteins are provided. Such methods include administering a therapeutically effective amount of at least one disclosed mutated FGF1 protein and/or at least one FGF1-vagus targeting chimera protein (such as at least 0.01 mg/kg, at least 0.05 mg/kg, at least 0.1 mg/kg, or at least 0.5 mg/kg) (or nucleic acid molecules encoding such) (such as 2, 3, 4 or 5 disclosed mutated FGF1 proteins and FGF1-vagus targeting chimera proteins) to reduce blood glucose in a mammal, such as a decrease of at least 5%, at least 10%, at least 25% or at least 50%, for example as compared to administration of no mutant FGF1 mutant protein or FGF1-vagus targeting chimera protein (e.g., administration of PBS).

In one example, the method is a method of reducing fed and fasting blood glucose, improving insulin sensitivity and glucose tolerance, reducing systemic chronic inflammation, ameliorating hepatic steatosis in a mammal, reducing triglycerides, decreasing insulin resistance, reducing hyperinsulinemia, increasing glucose tolerance, reducing hyperglycemia, reducing food intake, or combinations thereof. Such a method can include administering a therapeutically effective amount of one or more disclosed mutated FGF1 proteins (such as at least 0.01 mg/kg, at least 0.05 mg/kg, at least 0.1 mg/kg, or at least 0.5 mg/kg), one or more disclosed FGF1-vagus-targeting chimeric proteins (such as at least 0.01 mg/kg, at least 0.05 mg/kg, at least 0.1 mg/kg, at least 0.5 mg/kg, at least 0.63 mg/kg, or at least 1 mg/kg), or nucleic acid molecules encoding such proteins, to reduce fed and fasting blood glucose, improve insulin sensitivity and glucose tolerance, reduce systemic chronic inflammation, ameliorate hepatic steatosis in a mammal, reduce food intake, or combinations thereof.

In one example, the method is a method of treating a metabolic disease (such as metabolic syndrome, diabetes, or obesity) in a mammal. Such a method can include administering a therapeutically effective amount of one or more disclosed mutated FGF1 proteins (such as at least 0.01 mg/kg, at least 0.05 mg/kg, at least 0.1 mg/kg, or at least 0.5 mg/kg), one or more disclosed FGF1-vagus-targeting chimeric proteins (such as at least 0.01 mg/kg, at least 0.05 mg/kg, at least 0.1 mg/kg, at least 0.5 mg/kg, at least 0.63 mg/kg, or at least 1 mg/kg), or nucleic acid molecules encoding such proteins, to treat the metabolic disease.

In some examples, the mammal, such as a human, cat, or dog, has diabetes. Methods of administration are routine, and can include subcutaneous, intraperitoneal, intramuscular, or intravenous injection or infusion. In some examples, the mutated FGF1 protein is a mutated canine FGF1 protein, and is used to treat a dog. For example, a canine FGF1 (such as XP_849274.1) can be mutated to include one or more of the mutations disclosed herein, such as an N-terminal deletion or one or more point mutations shown in Table 1. Similarly, in some embodiments, the mutated FGF1 protein is a mutated cat FGF1 protein, and is used to treat a cat. Thus, for example, a feline FGF1 (such as XP_011281008.1) can be mutated to include one or more of the mutations disclosed herein, such as an N-terminal deletion or one or more point mutations shown in Table 1. Based on routine methods of sequence alignment (e.g., see FIG. 1), one skilled in the art can mutate any known FGF1 sequence to generate mutations that correspond to those provided herein (for example, the FGF1 sequence can be selected based on the subject to be treated, e.g., a dog can be treated with a mutated canine FGF1 protein or corresponding nucleic acid molecule). Such mutated FGF1 proteins can be part of an FGF1-vagus targeting chimera disclosed herein.

In some examples, use of the FGF1 mutants and/or FGF1-vagus targeting chimeras disclosed herein does not lead to (or significantly reduces, such as a reduction of at least 20%, at least 50%, at least 75%, or at least 90%) the adverse side effects observed with thiazolidinediones (TZDs) therapeutic insulin sensitizers, including weight gain, increased liver steatosis and bone fractures (e.g., reduced effects on bone mineral density, trabecular bone architecture and cortical bone thickness).

Provided are methods of reducing fed and fasting blood glucose, improving insulin sensitivity and glucose tolerance, reducing systemic chronic inflammation, ameliorating hepatic steatosis, reducing food intake, or combinations thereof, in a mammal, such as within 12 hours, within 24 hours, or within 48 hours of the treatment, such as within 12 to 24 hours, within 12 to 36 hours, or within 24 to 48 hours. In some examples, such treatment reduces blood glucose for at least 2 hours, at least 4 hours, at least 12 hours, at least 24 hours, at least 48 hours, at least 5 days, at least 7 days, at least 14 days, at least 21 days, at least 30 days, at least 34 days, or longer. Such methods can include administering a therapeutically effective amount of a FGF1 mutant and/or FGF1-vagus targeting chimera disclosed herein, to the mammal, or a nucleic acid molecule encoding the FGF1 mutant and/or FGF1-vagus targeting chimera or a vector comprising the nucleic acid molecule, thereby reducing fed and fasting blood glucose, improving insulin sensitivity and glucose tolerance, reducing systemic chronic inflammation, ameliorating hepatic steatosis, reducing one or more non-HDL lipid levels, reducing food intake, or combinations thereof, in a mammal. In some examples, the fed and fasting blood glucose is reduced in the treated subject by at least 10%, at least 20%, at least 30%, at least 50%, at least 75%, or at least 90% as compared to an absence of administration of the FGF1 mutant and/or FGF1-vagus targeting chimera. In some examples, insulin sensitivity and glucose tolerance is increased in the treated subject by at least 10%, at least 20%, at least 30%, at least 50%, at least 75%, or at least 90% as compared to an absence of administration of the FGF1 mutant and/or FGF1-vagus targeting chimera. In some examples, systemic chronic inflammation is reduced in the treated subject by at least 10%, at least 20%, at least 30%, at least 50%, at least 75%, or at least 90% as compared to an absence of administration of the FGF1 mutant and/or the FGF1-vagus targeting chimera. In some examples, hepatic steatosis is reduced in the treated subject by at least 10%, at least 20%, at least 30%, at least 50%, at least 75%, or at least 90% as compared to an absence of administration of the FGF1 mutant and/or the FGF1-vagus targeting chimera. In some examples, one or more lipids (such as a non-HDL, for example IDL, LDL and/or VLDL) are reduced in the treated subject by at least 10%, at least 20%, at least 30%, at least 50%, at least 75%, or at least 90% as compared to an absence of administration of the FGF1 mutant and/or the FGF1-vagus targeting chimera. In some examples, triglyceride and or cholesterol levels are reduced with the FGF1 mutant by at least 10%, at least 20%, at least 30%, at least 50%, at least 75%, or at least 90% as compared to an absence of administration of the FGF1 mutant and/or the FGF1-vagus targeting chimera. In some examples, the amount of food intake is reduced in the treated subject by at least 10%, at least 20%, at least 30%, at least 50%, at least 75%, or at least 90% as compared to an absence of administration of the FGF1 mutant (such as within 12 hours, within 24 hours, or within 48 hours of the treatment, such as within 12 to 24 hours, within 12 to 36 hours, or within 24 to 48 hours). In some examples, combinations of these reductions are achieved.

### Mutated FGF1 Proteins and FGF1-Vagus Targeting Chimeric Proteins

The present disclosure provides mutated FGF1 proteins, as well as FGF1-vagus targeting chimeric proteins that include such a mutated FGF1 protein. However, in some examples, such a chimera includes a native FGF1 protein, such as SEQ ID NO: 5. FGF1 mutants include an N-terminal deletion, one or more point mutations (such as amino acid substitutions, deletions, additions, or combinations thereof), or combinations of N-terminal deletions and one or more additional point mutations. The disclosed mutated FGF1 proteins and FGF1-vagus targeting chimeric proteins, and corresponding coding sequences, can be used in the methods provided herein.

### FGF1

FGF1 (such as SEQ ID NOS: 2, 4, 5, 6, 7, 8, or 9) can be mutated to include mutations to control (e.g., reduce) the mitogenicity of the protein and to provide glucose-lowering ability to the protein. Mutations can also be introduced to affect the stability and receptor binding selectivity of the protein. Such mutant FGF1 proteins (such as those provided herein, as well as those provided in PCT/US2014/061638, PCT/US2016/028368, and PCT/US2016/028365) can be used as part of an FGF1-vagus targeting chimeric protein.

FIG. 1 shows an alignment between different mammalian wild-type FGF1 sequences: human (SEQ ID NO: 2), gorilla (SEQ ID NO: 6), chimpanzee (SEQ ID NO: 7), canine (SEQ ID NO: 8), feline (SEQ ID NO: 8), and mouse (SEQ ID NO: 4). In some examples, FGF1 includes SEQ ID NO: 2, 4, 6, 7 or 8, but without the N-terminal methionine (resulting in a 154 aa FGF1 protein). In addition, the mature/active form of FGF1 is one where a portion of the N-terminus is removed, such as the N-terminal 15, 16, 20, or 21 amino acids from SEQ ID NO: 2, 4, 6, 7 or 8. Thus, in some examples the active form of FGF1 comprises or consists of amino acids 16-155 or 22-155 of SEQ ID NOS: 2 or 4 (e.g., see SEQ ID NO: 5). In some examples, the mature form of FGF1 that can be mutated includes SEQ ID NO: 5 with a methionine added to the N-terminus (wherein such a sequence can be mutated as discussed herein). Thus, a mutated mature FGF1 protein can include an N-terminal truncation.

In some examples, multiple types of mutations disclosed herein are made to an FGF1 protein. Although mutations below are noted by a particular amino acid for example in SEQ ID NOS: 2, 4, or 5, one skilled in the art will appreciate that the corresponding amino acid can be mutated in any FGF1 sequence (for example by using the alignment shown in FIG. 1, or by generating a similar alignment for the FGF1 of interest). For example, Q40 of SEQ ID NO: 5 corresponds to Q55 of SEQ ID NOS: 2 and 4.

In some examples, the mutant FGF1 is a truncated version of the mature protein (e.g., SEQ ID NO: 5), which can include for example deletion of at least 5, at least 6, at least 9, at least 10, at least 11, at least 12, at least 13, at least 14, at least 15, or at least 20 consecutive N-terminal amino acids. Thus, in some examples, the mutant FGF1 protein is a truncated version of the mature protein (e.g., SEQ ID NO: 5), such a deletion of the N-terminal 5, 6, 7, 8, 9, 10, 11, 12, 13, 14, 15, 16, 17, 18, 19 or 20 amino acids shown in SEQ ID NO: 5. For example, mutations can be made to the N-terminal region of FGF1 (such as SEQ ID NOS: 2, 4, or 5), such as deletion of the first 14, 15, 16, 17, 18, 19, 20, 21, 22, 23, 24, 25, 26, 27, 28, 29, or 30 amino acids of SEQ ID NOS: 2 or 4 (such as deletion of at least the first 14 amino acids of SEQ ID NO: 2 or 4, such as deletion of at least the first 15, at least 16, at least 20, at least 25, or at least 29 amino acids of SEQ ID NOS: 2 or 4), deletion of the first 8, 9, 10, 11, 12, 13, 14, 15, 16, 17, 18, 19, or 20 amino acids of SEQ ID NO: 5 (e.g., see SEQ ID NOS: 13-24). In some examples, the FGF1 mutant includes an N-terminal deletion, but retains a methionine at the N-terminal position. In some examples, an N-terminally deleted FGF1 protein has reduced mitogenic activity as compared to wild-type mature FGF1 protein. In some examples, an N-terminally deleted FGF1 protein has amino acids added back to the N-terminus, such as adding the sequence MRDSSPL (referred to herein as NF21), for example as shown in SEQ ID NOS: 13-15 and 20-21, or a sequence that binds FGF1Rb (e.g., see SEQ ID NOS: 420, 421 and 422). In some examples, such an N-terminally deleted FGF1 protein has reduced mitogenic activity as compared to wild-type mature FGF1 protein (e.g., see SEQ ID NO:5).

Thus, in some examples, the mutant FGF1 protein includes at least 90 consecutive amino acids from amino acids 5-141 or 5-155 of FGF1 (e.g., of SEQ ID NOS: 2 or 4), (which in some examples can include further deletion of N-terminal amino acids 1-20 and/or point mutations, such as substitutions, deletions, and/or additions). In some examples, the mutant FGF1 protein includes at least 90 consecutive amino acids from amino acids 1-140 of FGF1 (e.g., of SEQ ID NO: 5), (which in some examples can include further deletion of N-terminal amino acids 1-20 and/or point mutations, such as substitutions, deletions, and/or additions). Thus, in some examples, the mutant FGF1 protein includes at least 90 consecutive amino acids from amino acids 5-141 of FGF1, such as at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 115, at least 120, at least 125, or at least 130 consecutive amino acids from amino acids 5-141 of SEQ ID NOS: 2 or 4 (such as 90-115, 90-125, 90-100, or 90-95 consecutive amino acids from amino acids 5-141 of SEQ ID NOS: 2 or 4). In some examples, the mutant FGF1 protein includes least 90 consecutive amino acids from SEQ ID NO: 5. Thus, in some examples, the mutant FGF1 protein includes at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, or at least 110 consecutive amino acids from SEQ ID NO: 5 (such as 90-115, 90-100, or 90-95 consecutive amino acids from SEQ ID NO: 5).

In some examples, the mutant FGF1 protein includes at least 1, at least 4, at least 5, at least 6, at least 7, at least 8, at least 9, at least 10, at least 11, at least 12, at least 13, at least 14, at least 15, at least 16, at least 17, at least 18, at least 19, or at least 20 additional amino acid substitutions, such as 1-20, 1-10, 4-8, 5-12, 5-10, 5-25, 1, 2, 3, 4, 5, 6, 7, 8, 9, 10, 11, 12, 13, 14, or 15 additional amino acid substitutions. For example, point mutations can be introduced into an FGF1 sequence to decrease mitogenicity, increase stability, alter binding affinity for heparin and/or heparan sulfate (compared to the portion of a native FGF1 protein without the modification), or combinations thereof. Specific exemplary point mutations that can be used are shown above in Table 1.

In some examples, the mutant FGF1 protein includes one or more mutations (such as a substitution or deletion) at one or more of the following positions: Y8, K9, K10, K12, L14, Y15, C16, S17, N18, H21, R35, Q40, Q43, L44, L46, S47, E49, Y55, A66, M67, L73, C83, E87, R88, H93, Y94, N95, S99, K101, H102, A103, E104, K105, N106, W107, F108, V109, L111, K112, K113, N114, 5116, C117, K118, R119, G120, P121, R122, Q127, F132, L133, P134, and L135, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or 54 of these positions. In some examples the mutant FGF1 protein has as one or more of Y8F, Y8V, Y8A, K9T, K9R, K9A, K10T, K12V, L14A, Y15F, Y15A, Y15V, C16V, C16A, C16T, C16S, S17R, S17K, N18R, N18K, H21Y, R35E, R35V, R35K, Q40P, Q43K, Q43E, Q43A, L44F, L46V, S471, S47A, S47V, E49D, E49K, E49Q, E49A, Y55F, Y55V, Y55S, Y55A, Y55W, A66C, M67I, L73V, C83T, C83S, C83A C83V, E87V, E87A, E87S, E87T, E87Q, E87D, E87H, R88Y, R88L, R88D, H93G, H93A, Y94V, Y94F, Y94A, N95V, N95A, N95S, N95T, S99A, K101E, H102Y, H102A, A103G, Δ104-106, W107A, F108Y, V109L, L111I, K112D, K112E, K112Q, K113Q, K113E, K113D, N114K, N114R, S116R, C117V, C117P, C117T, C117S, C117A, K118N, K118E, K118V, R119G, R119V, R119E, Δ120-122, Q127R, Q127K, F132W, L133A, L133V, L133S, P134V, L135A, and L135S (wherein the numbering refers to SEQ ID NO: 5), such as 1 to 3, 1 to 4, 1 to 5, 1 to 6, 1 to 7, 1 to 8, 1 to 10, 2 to 5, 2 to 10, 3 to 6, or 2 to 8 of these mutations, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or 54 of these mutations.

Mutant FGF1 proteins can include both an N-terminal deletion and one or more point mutations, such as those shown in Table 1. Thus, any of SEQ ID NOS: 13, 14, 15, 20, 21, 420, 421, can 422 can be modified to include one or more of the point mutations shown in Table 1.

Specific exemplary FGF1 mutant proteins are shown in SEQ ID NOS: 10-422, such as SEQ ID NO: 420, 421, or 422. One skilled in the art will recognize that variations can be made to these sequences, without adversely affecting the function of the protein (such as its ability to reduce blood glucose). In some examples, an FGF1 mutant protein includes at least 80% sequence identity to any of SEQ ID NOS: 10-422, such as at least 80% sequence identity to SEQ ID NO: 420, 421, or 422. Thus, a FGF1 mutant protein can have at least 85%, at least 90%, at least 95%, at least 96%, at least 97%, at least 98% or at least 99% sequence identity to any of SEQ ID NOS: 10-422, such as at least such sequence identity to SEQ ID NO: 420, 421, or 422, and retains the ability to treat a metabolic disease and/or decrease blood glucose in a mammal (such as a mammal with type II diabetes), and in some example retains the N-terminal deletion and/or point mutation(s) noted herein for each particular SEQ ID NO:. However, such variants are not a native FGF1 sequence, e.g., SEQ ID NO: 5. In some examples, the FGF1 mutant protein includes or consists of any of SEQ ID NOS: 10-422, such as SEQ ID NO: 420, 421, or 422. The disclosure encompasses variants of the disclosed FGF1 mutant proteins, such as variants of any of SEQ ID NOS: 10-422, such as SEQ ID NO: 420, 421, or 422, having 1 to 20, 1 to 15, 1 to 10, 1 to 8, 2 to 10, 1 to 5, 1 to 6, 2 to 12, 3 to 12, 5 to 12, or 5 to 10 additional mutations, such as conservative amino acid substitutions.

In some examples, the mutant FGF1 protein has at its N-terminus a methionine. In some examples, the mutant FGF1 protein is at least 120 amino acids in length, such as at least 125, at least 130, at least 135, at least 140, at least 145, at least 150, at least 155, at least 160, or at least 175 amino acids in length, such as 120-160, 125-160, 130-160, 150-160, 130-200, 130-180, 130-170, or 120-160 amino acids in length.

In some examples, the disclosed FGF1 mutant proteins have reduced mitogenicity compared to mature native FGF1 (e.g., SEQ ID NO: 5), such as a reduction of at least 20%, at least 50%, at least 75% or at least 90%.

In one example, the disclosed FGF1 mutant proteins have improved thermostability compared to mature native FGF1 (e.g., SEQ ID NO: 5), such as an increase of at least 10%, at least 20%, at least 50%, or at least 75% (e.g., see Xia et al., PLoS One. 2012; 7(11):e48210 and Zakrzewska, J Biol Chem. 284:25388-25403, 2009). Methods of measuring FGF1 stability are known in the art, such as measuring denaturation of FGF1 or mutants by fluorescence and circular dichroism in the absence and presence of a 5-fold molar excess of heparin in the presence of 1.5 M urea or isothermal equilibrium denaturation by guanidine hydrochloride. In one example, the assay provided by Dubey et al., J. Mol. Biol. 371:256-268, 2007 is used to measure FGF1 stability.

In one example, the disclosed FGF1 mutant proteins have improved protease resistance compared to mature native FGF1 (e.g., SEQ ID NO: 5), such as an increase of at least 10%, at least 20%, at least 50%, or at least 75% (e.g., see Kobielak et al., Protein Pept Lett. 21(5):434-43, 2014).

In some examples, an FGF1 mutant protein includes mutations that increase its blood glucose lowering ability relative to the mature wild-type FGF1 (e.g., SEQ ID NO: 5), such as an increase of at least 10%, at least 20%, at least 50%, at least 75%, or at least 90%. In some examples, the FGF1 mutant protein has a similar glucose lowering to mature wild-type FGF1 (e.g., SEQ ID NO: 5). Methods of measuring blood glucose are known and are provided herein.

In some examples, a mutated FGF1 includes one or more mutations that increase the thermostability (e.g., relative to mature or truncated FGF1, e.g., SEQ ID NO: 5), such as an increase of at least 20%, at least 50%, at least 75% or at least 90% compared to native FGF1. Exemplary mutations that can be used to increase the thermostability include, but are not limited to, (a) one or more of C117V, A66C, K12V, and N95V, (b) one or more of C117V, Y55W, E87H, and S116R, (c) one or more of C117V, S116R, K12V, N95V, and Y55W, (d) one or more of K12V, L44F, C83T, N95V, C117V, and F132W, (e) one or more of K12V, H21Y, L44F, N95V, H102Y, F108Y, and C117V (f) one or more of K12V, E87V, and C117V, (g) one or more of Q40P, S47I, H93G, and N95V, (h) one or more of H21Y, L44F, H102Y, F108Y, and N95V, (i) one or more of H21Y, L44F, H102Y, F108Y, N95V and C117V, (j) one or more of L44F, C83T, N95V, C117V, and F132W, (k) one or more of Q40P, S47I, H93G, K12V, and N95V, (k) one or more of H21Y, L44F, H102Y, F108Y, K12V, and N95V, or (k) one or more of K12V and N95V, wherein the numbering refers to SEQ ID NO: 5. For example, a mutated FGF1 can be mutated to increase the thermostability of the protein relative to an FGF1 protein without the modification. Methods of measuring thermostability are known in the art. In one example, the method provided in Xia et al., PloS One. 7:e48210, 2012 is used.

Mutations can be made to a mutant FGF1 (such as to any of SEQ ID NOS: 10-422, such as SEQ ID NO: 420, 421, or 422) to reduce its mitogenic activity (e.g., relative to the mature wild-type FGF1, e.g., SEQ ID NO: 5). In some examples, such mutations reduce mitogenic activity by at least 20%, at least 50%, at least 60%, at least 70%, at least 75%, at least 80%, at least 90%, at least 92%, at least 95%, at least 98%, at least 99%, or even complete elimination of detectable mitogenic activity, as compared to a native FGF1 protein without the mutation. In some examples, the FGF1 mutant protein has an EC₅₀ for mitogenicity that is shifted by several orders of magnitude relative to the mature wild-type FGF1 (e.g., SEQ ID NO: 5) (such as an EC₅₀ increase of 1 log, 2 logs, or 3 logs), or even no detectable mitogenicity. Methods of measuring mitogenicity are known in the art and are provided herein. Examples include thymidine incorporation into DNA in serum-starved cells (e.g., NIH 3T3 cells) stimulated with the mutated FGF1, methylthiazoletetrazolium (MTT) assay (for example by stimulating serum-starved cells with mutated FGF1 for 24 hr then measuring viable cells), cell number quantification or BrdU incorporation. In some examples, the assay provided by Fu et al., World J. Gastroenterol. 10:3590-6, 2004; Klingenberg et al., J. Biol. Chem. 274:18081-6, 1999; Shen et al., Protein Expr Purif 81:119-25, 2011, or Zou et al., Chin. Med. J. 121:424-429, 2008 is used to measure mitogenic activity.

Mutations that reduce the heparan binding affinity (such as a reduction of at least 10%, at least 20%, at least 50%, or at least 75%, e.g., as compared to a native FGF1 protein without the mutation), can also be used to reduce mitogenic activity, for example by substituting heparan binding residues from a paracrine FGFs into a mutant FGF1.

In some examples, an FGF1 mutant includes mutations to the FGF1 nuclear export sequence, for example to decrease the amount of FGF1 in the nucleus and reduce its mitogenicity as measured by thymidine incorporation assays in cultured cells (e.g., see Nilsen et al., J. Biol. Chem. 282(36):26245-56, 2007). Mutations to the nuclear export sequence decrease FGF1-induced proliferation (e.g., see Nilsen et al., J. Biol. Chem. 282(36):26245-56, 2007). Methods of measuring FGF1 degradation are known in the art, such as measuring [³⁵S]methionine-labeled FGF1 or immunoblotting for steady-state levels of FGF1 in the presence or absence of proteasome inhibitors. In one example, the assay provided by Nilsen et al., J. Biol. Chem. 282(36):26245-56, 2007 or Zakrzewska et al., J. Biol. Chem. 284:25388-403, 2009 is used to measure FGF1 degradation.

In some examples, the mutant FGF1 protein includes mutations at 1, 2, 3, or 4, of the following positions: K12, A66, N95, and C117 (wherein the numbering refers to SEQ ID NO: 5), such as one or more of K12V, A66C, N95V, and C117V, (such as 1, 2, 3, or 4 of these mutations).

In some examples, the mutant FGF1 protein includes mutations at 1, 2, 3, or 4, of the following positions: S99, K101, H102, and W107 (wherein the numbering refers to SEQ ID NO: 5), such as one or more of S99A, K101E, H102A, and W107A, (such as 1, 2, 3, or 4 of these mutations).

In one example, the mutant FGF1 protein includes a mutation at E87 or N95, such as replacement with a non-charged amino acid.

In one example, the mutant FGF1 protein includes K12V, H21Y, L44F, N95V, H102Y, F108Y, and C117V mutations.

In some examples, the mutant FGF1 protein includes a mutation at K12 of FGF1, which is predicted to be at the receptor interface. Thus, K12 of SEQ ID NO: 5 can be mutated, for example to a V or C.

### Vagus Nerve Targeting Peptides

The vagus nerve is the tenth cranial nerve, and interfaces with parasympathetic control of the heart, lungs, and digestive tract. The inventors have found that peptides or proteins that target the vagus nerve (such as those that allow binding of the chimera to the vagus nerve) can be attached to an FGF1 protein, such as the mutant FGF1 sequences provided herein, to achieve a longer glucose lowering effect in vivo.

Examples of peptides that target the vagus nerve include glucagon-like peptide (GLP1) (e.g., SEQ ID NO: 437 or 438) (see for example Imeryuz et al., Am J. Physiol. 273:920-G927, 1997), GLP1 receptor agonists, and GLP1 analogs that are resistant to digestion by dipeptidyl peptidase IV, such as exendin 4 (SEQ ID NO: 423) or a truncated version thereof (e.g., SEQ ID NO: 434). In one example the vagus targeting peptide is oxyntomodulin (e.g., SEQ ID NO: 425) or peptide YY (e.g., SEQ ID NO: 426, see for example Abbott et al., Brain Res., 1044:127-31, 2005). In some examples, peptide YY (e.g., SEQ ID NO: 426) further includes amino acids YP at the N-terminus, or start with AK instead of IK at the N-terminus.

Thus, in some examples, the vagus nerve targeting protein includes at least 80% sequence identity to SEQ ID NO: 423, 434, 435, 436, 437 or 438, such as at least 85%, at least 90%, at least 95%, at least 96%, at least 97%, at least 98% or at least 99% sequence identity to any of SEQ ID NOS: 423, 434, 435, 436, 437 or 438, and retains the ability to target the chimeric peptide to the vagus nerve.

Thus, in some examples, the vagus nerve targeting portion includes dulaglutide, liraglutide, lixisenatide, and/or albiglutide.

### FGF1-Vagus Targeting Chimeric Proteins

FGF1-vagus targeting chimeric proteins are provided that include an FGF1 portion (such as a mutant FGF1) and a vagus nerve peptide portion. Such proteins can be used in the methods provided herein.

Thus, the FGF1 portion of the chimeric protein can be any mutant FGF1 protein provided herein, such as SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, or a sequence having at least 80%, at least 90%, at least 95%, at least 96%, at least 97%, at least 98% or at least 99% sequence identity to SEQ ID NOS: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, while retaining the ability to lower blood glucose. In some examples, such an FGF1 mutant protein retains the N-terminal deletion/modification and/or point mutation(s) provided herein for that sequence (e.g., for SEQ ID NO: 408, a mutant FGF1 variant having at least 95% sequence identity to SEQ ID NO: 408 would retain the Y55A, S116R, and C117V mutations).

In some examples, the peptide that targets the vagus nerve includes glucagon-like peptide (GLP1) (e.g., SEQ ID NO: 437 or 438), GLP1 receptor agonists, and GLP1 analogs that are resistant to digestion by dipeptidyl peptidase IV, such as exendin 4 (SEQ ID NO: 423) or a truncated version thereof (e.g., SEQ ID NO: 434). In one example the vagus targeting peptide is oxyntomodulin (e.g., SEQ ID NO: 425) or peptide YY (e.g., SEQ ID NO: 426). In some examples, peptide YY (e.g., SEQ ID NO: 426) further includes amino acids YP at the N-terminus, or start with AK instead of IK at the N-terminus.

Thus, in some examples, the vagus nerve targeting protein includes at least 80% sequence identity to SEQ ID NO: 423, 434, 435, 436, 437 or 438, such as at least 85%, at least 90%, at least 95%, at least 96%, at least 97%, at least 98% or at least 99% sequence identity to any of SEQ ID NOS: 423, 434, 435, 436, 437 or 438, and retains the ability to target the chimeric peptide to the vagus nerve.

Specific examples of FGF1 mutant proteins that can form the FGF1 portion of the chimera are shown in the right hand column of Table 2. Specific examples of vagus nerve targeting proteins that can form the vagus targeting portion of the chimera are shown in the left hand column of Table 2. Thus any FGF1 mutant protein in Table 2 and be combined with any vagus targeting protein in Table 2, to form an FGF1-vagus targeting chimeric protein. In some examples, the FGF1 portion of the chimera is at the N-terminus of the chimera, and the vagus nerve targeting protein portion is the C-terminus of the chimera. However, this can be reversed, such that the FGF1 portion of the chimera is the C-terminus of the chimera, and the vagus nerve targeting protein portion is the N-terminus of the chimera. In some examples, the FGF1 (e.g., mutant FGF1) and vagus nerve targeting protein portion are linked indirectly through the use of a linker, such as one composed of at least 5, at least 10, at least 15 or at least 20 amino acids, such as 5, 6, 7, 8, 9, 10, 11, 12, 13, 14, 15, 16, 17, 18, 19 or 20 amino acids. In some examples the linker is flexible. In one example the linker is a polyalanine. In one example, the linker is GSGSGS. In some examples, the FGF1-vagus targeting chimeric protein includes or consists of SEQ ID NO: 424, 425, 426, 427, 428, 429, 430, 431, 432, or 433. The disclosure encompasses variants of the disclosed FGF1-vagus targeting chimeric proteins, such as SEQ ID NO: 424, 425, 426, 427, 428, 429, 430, 431, 432, or 433, having 1 to 8, 2 to 10, 1 to 5, 1 to 6, or 5 to 10 mutations, such as conservative amino acid substitutions. In some examples, a FGF1-vagus targeting chimeric protein includes at least 80%, at least 90%, at least 95%, at least 96%, at least 97%, at least 98% or at least 99% sequence identity to SEQ ID NO: 424, 425, 426, 427, 428, 429, 430, 431, 432, or 433, while retaining the ability to lower blood glucose and/or treat a metabolic disorder, such as type 2 diabetes.

TABLE 2
Exemplary Proteins to Make an FGF1-Vagus Targeting Chimera

| Exemplary | |
|---|---|
| Vagus Nerve Targeting Peptides | Exemplary FGF1 Peptides |
| HGEGTFTSDL SKQMEEEAVR | FNLPPGNYKK PVLLYCSNGG HFLRILPDGT VDGTRDRSDQ HIQLQLSAES |
| LFIEWLKNGG PSSGAPPPS (SEQ | VGEVYIKSTE TGQYLCMDTD GLLYGSQTPN EECLFLERLE ENHYVTYISK |
| ID NO: 423) | KHAEKNWFVG LKKNGSVKRG PRTHYGQKAI LFLPLPVSSD (SEQ ID NO: 10) |
| DLSKQMEEEAVR LFIEWLKNGG | FNLPPGNYKK PKLLYCSNGG HFLRILPDGT VDGTRDRSDQ HIQLQLSAES |
| PSSGAPPPS (SEQ ID NO: 434) | VGEVWIKSTE TGQYLAMDTD GLLYGSQTPN EECLFLHRLE ENHYNTYISK |
| | KHAEKNWFVG LKKNGRVKRG PRTHYGQKAI LFLPLPVSSD (SEQ ID NO: 11) |
| HSEGTFTS DYSKYLDSRR AQDFVQWLMN | FNLPPGNYKK PVLLYCSNGG HFLRILPDGT VDGTRDRSDQ HIQLQLSAES |
| TKRNRNNIA (SEQ ID NO: 435) | VGEVWIKSTE TGQYLAMDTD GLLYGSQTPN EECLFLERLE ENHYVTYISK |
| | KHAEKNWFVG LKKNGRVKRG PRTHYGQKAI LFLPLPVSSD (SEQ ID NO: 12) |
| IKPEA PGEDASPEEL NRYYASLRHY | MRDSSPL PVLLYCSNGG HFLRILPDGT VDGTRDRSDQ HIQFQLSAES |
| LNLVTRQRY | VGEVYIKSTE TGQYLAMDTD GLLYGSQTPN EETLFLERLE ENHYVTYISK |
| (SEQ ID NO: 436) | KHAEKNWFVG LKKNGSVKRG PRTHYGQKAI LWLPLPVSSD (SEQ ID NO: 13) |
| HAEGTFTSDVSSYLEGQAAKEFIAWLVKG | MRDSSPL PVLLYCSNGG YFLRILPDGT VDGTRDRSDQ HIQFQLSAES |
| RG (SEQ ID NO: 437) | VGEVYIKSTE TGQYLAMDTD GLLYGSQTPN EECLFLERLE ENHYVTYISK |
| | KYAEKNWYVG LKKNGSVKRG PRTHYGQKAI LFLPLPVSSD (SEQ ID NO: 14) |
| HAEGTFTSDVSSYLEGQAAKEFIAWLVKG | MRDSSPL PVLLYCSNGG HFLRILPDGT VDGTRDRSDQ HIQLQLSAES |
| R-NH₂; (SEQ ID NO: 438) | VGEVYIKSTE TGQYLAMDTD GLLYGSQTPN EECLFLVRLE ENHYNTYISK |
| | KHAEKNWFVG LKKNGSVKRG PRTHYGQKAI LFLPLPVSSD (SEQ ID NO: 15) |
| | LYCSNGG HFLRILPDGT VDGTRDRSDP HIQLQLIAES VGEVYIKSTE |
| | TGQYLAMDTD GLLYGSQTPN EECLFLERLE ENGYVTYISK KHAEKNWFVG |
| | LKKNGSCKRG PRTHYGQKAI LFLPLPVSSD (SEQ ID NO: 16) |
| | LYCSNGG YFLRILPDGT VDGTRDRSDQ HIQFQLSAES VGEVYIKSTE |
| | TGQYLAMDTD GLLYGSQTPN EECLFLERLE ENHYVTYISK KYAEKNWYVG |
| | LKKNGSCKRG PRTHYGQKAI LFLPLPVSSD (SEQ ID NO: 17) |
| | LYCSNGG VFLRILPDGT VDGTRDRSDQ HIQFQLSAES VGEVYIKSTE |
| | TGQYLAMDTD GLLYGSQTPN EECLFLERLE ENHYVTYISK KYAEKNWYVG |
| | LKKNGSVKRG PRTHYGQKAI LFLPLPVSSD (SEQ ID NO: 18) |
| | LYCSNGG HFLRILPDGT VDGTRDRSDQ HIQFQLSAES VGEVYIKSTE |
| | TGQYLCMDTD GLLYGSQTPN EETLFLERLE ENHYVTYISK KHAEKNWFVG |
| | LKKNGSVKRG PRTHYGQKAI LWLPLPVSSD (SEQ ID NO: 19) |
| | MRDS SPLLYCSNGG YFLRILPDGT VDGTRDRSDQ HIQFQLSAES VGEVYIKSTE |
| | TGQYLAMDTD GLLYGSQTPN EECLFLERLE ENHYVTYISK KYAEKNWYVG |
| | LKKNGSVKRG PRTHYGQKAI LFLPLPVSSD (SEQ ID NO: 20) |
| | MRDSSPLG GQVLYCSNGG YFLRILPDGT VDGTRDRSDQ HIQFQLSAES |
| | VGEVYIKSTE TGQYLAMDTD GLLYGSQTPN EECLFLERLE ENHYVTYISK |
| | KYAEKNWYVG LKKNGSVKRG PRTHYGQKAI LFLPLPVSSD (SEQ ID NO: 21) |
| | VLLYCSNGG HFLRILPDGT VDGTRDRSDP HIQLQLIAES VGEVYIKSTE |
| | TGQYLAMDTD GLLYGSQTPN EECLFLERLE ENGYVTYISK KHAEKNWFVG |
| | LKKNGSCKRG PRTHYGQKAI LFLPLPVSSD (SEQ ID NO: 22) |
| | VLLYCSNGG YFLRILPDGT VDGTRDRSDQ HIQFQLSAES VGEVYIKSTE |
| | TGQYLAMDTDGLLYGSQTPN EECLFLERLE ENHYVTYISK KYAEKNWYVG |
| | LKKNGSCKRG PRTHYGQKAI LFLPLPVSSD (SEQ ID NO: 23) |
| | VLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO: 24) |
| | FNLPPGNYKKPKLLYCSNGG HFLRILPDGT VDGTRDRSDQ HIQLQLSAES |
| | VGEVYIKSTE TGQYLAMDTD GLLYGSQTPN EECLFLERLE ENHYNTYIAK |
| | EAAEKNAFVG LKKNGSCKRG PRTHYGQKAI LFLPLPVSSD (SEQ ID NO: 25) |
| | PPGNYK KPKLLYCSNG GHFLRILPDG TVDGTRDRSD QHIQLQLSAE |
| | SVGEVYIKST ETGQYLAMDT DGLLYGSQTP NEECLFLERL EENHYNTYIS |
| | KKHAEKNWFVGLKKNGSCKR GPRTHYGQKA ILFLPLPVSSD |
| | (SEQ ID NO. 26) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 27) |
| | LYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 28) |
| | KLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 29) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO. 30) |
| | FNLPPGNYKK PVLLYCSNGG HFLRILPDGT VDGTRDRSDQ HIQLQVSAES |
| | VGEVYIKSTE TGQYLAMDTDGLLYGSQTPN EECLFLVRLE ENHYVTYISK |
| | KHAEKNWFVG LKKNGSCKRG PRTHYGQKAI LFLVLPVSSD |
| | (SEQ ID NO. 31) |
| | NYKK PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCNRG PRTHYGQKAI LFLPLPVSSD (SEQ ID NO. 32) |
| | NYKK PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCERG PRTHYGQKAI LFLPLPVSSD |
| | (SEQ ID NO. 33) |
| | GGQVKPKLLYCSNG GHFLRILPDG TVDGTRDRSD QHIQLQLSAE SVGEVYIKST |
| | ETGQYLAMDT DGLLYGSQTP NEECLFLERL EENHYNTYIS KKHAEKNWFV |
| | GLKKNGSCKR GPRTHYGQKA ILFLPLPVSSD (SEQ ID NO. 34) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLVLPVSSD (SEQ ID NO. 35) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSVKRG PRTHYGQKAI LFLVLPVSSD (SEQ ID NO. 36) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSVKRG PRTHYGQKAI LFLVLPVSSD (SEQ ID NO. 37) |
| | VLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSVKRG PRTHYGQKAI LFLVLPVSSD (SEQ ID NO. 38) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLVLPVSSD (SEQ ID NO. 39) |
| | VLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLVLPVSSD (SEQ ID NO. 40) |
| | FNLPPGNYKK PKLLYCSNGG HFLRILPDGT VDGTRDRSDQ HIQFQLSAES |
| | VGEVYIKSTE TGQYLAMDTD GLLYGSQTPN EETLFLERLE ENHYNTYISK |
| | KHAEKNWFVG LKKNGSVKRG PRTHYGQKAI LWLPLPVSSD (SEQ ID NO. 41) |
| | FNLPPGNYKK PKLLYCSNGG HFLRILPDGT VDGTRDRSDQ HIQFQLSAES |
| | VGEVYIKSTE TGQYLAMDTD GLLYGSQTPN EETLFLERLE ENHYVTYISK |
| | KHAEKNWFVG LKKNGSVKRG PRTHYGQKAI LWLPLPVSSD (SEQ ID NO. 42) |
| | FNLPPGNYKK PVLLYCSNGG HFLRILPDGT VDGTRDRSDQ HIQFQLSAES |
| | VGEVYIKSTE TGQYLAMDTD GLLYGSQTPN EETLFLERLE ENHYNTYISK |
| | KHAEKNWFVG LKKNGSVKRG PRTHYGQKAI LWLPLPVSSD |
| | (SEQ ID NO. 43) |
| | FNLPPGNYKK PVLLYCSNGG HFLRILPDGT VDGTRDRSDQ HIQFQLSAES |
| | VGEVYIKSTE TGQYLAMDTD GLLYGSQTPN EETLFLERLE ENHYVTYISK |
| | KHAEKNWFVG LKKNGSVKRG PRTHYGQKAI LWLPLPVSSD (SEQ ID NO. 44) |
| | K PKLLYCSNGG HFLRILPDGT VDGTRDRSDQ HIQFQLSAES VGEVYIKSTE |
| | TGQYLAMDTD GLLYGSQTPN EETLFLERLE ENHYNTYISK KHAEKNWFVG |
| | LKKNGSVKRG PRTHYGQKAI LWLPLPVSSD |
| | (SEQ ID NO. 45) |
| | KLLYCSNGG HFLRILPDGT VDGTRDRSDQ HIQFQLSAES VGEVYIKSTE |
| | TGQYLAMDTD GLLYGSQTPN EETLFLERLE ENHYNTYISK KHAEKNWFVG |
| | LKKNGSVKRG PRTHYGQKAI LWLPLPVSSD (SEQ ID NO. 46) |
| | K PKLLYCSNGG HFLRILPDGT VDGTRDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 47) |
| | KLLYCSNGG HFLRILPDGT VDGTRDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 48) |
| | K PVLLYCSNGG HFLRILPDGT VDGTRDRSDQ HIQFQLSAES VGEVYIKSTE |
| | TGQYLAMDTD GLLYGSQTPN EETLFLERLE ENHYNTYISK KHAEKNWFVG |
| | LKKNGSVKRG PRTHYGQKAI LWLPLPVSSD (SEQ ID NO. 49) |
| | VLLYCSNGG HFLRILPDGT VDGTRDRSDQ HIQFQLSAES VGEVYIKSTE |
| | TGQYLAMDTD GLLYGSQTPN EETLFLERLE ENHYNTYISK KHAEKNWFVG |
| | LKKNGSVKRG PRTHYGQKAI LWLPLPVSSD (SEQ ID NO. 50) |
| | K PVLLYCSNGG HFLRILPDGT VDGTRDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 51) |
| | VLLYCSNGG HFLRILPDGT VDGTRDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 52) |
| | FNLPPGNYKK PKLLYCSNGG HFLRILPDGT VDGTRDRSDQ HIQFQLSAES |
| | VGEVYIKSTE TGQYLAIDTD GLVYGSQTPN EECLFLERLE ENHYNTYISK |
| | KHGWFLG IKKNGSVKGTHYGQKAI LFLPLPVSSD (SEQ ID NO. 53) |
| | FNLPPGNYKK PVLLYCSNGG HFLRILPDGT VDGTRDRSDQ HIQFQLSAES |
| | VGEVYIKSTE TGQYLAIDTD GLVYGSQTPN EECLFLERLE ENHYNTYISK |
| | KHGWFLGIKKNGSVKGTHYGQKAI LFLPLPVSSD (SEQ ID NO. 54) |
| | FNLPPGNYKK PVLLYCSNGG HFLRILPDGT VDGTRDRSDQ HIQFQLSAES |
| | VGEVYIKSTE TGQYLAIDTD GLVYGSQTPN EECLFLERLE ENHYVTYISK |
| | KHGWFLG IKKNGSVKGTHYGQKAI LFLPLPVSSD (SEQ ID NO. 55) |
| | FNLPPGNYKK PVLLYCSNGG HFLRILPDGT VDGTRDRSDQ HIQFQLSAES |
| | VGEVYIKSTE TGQYLAIDTD GLVYGSQTPN EECLFLERLE ENHYVTYISK |
| | KHGWFLG IKKNGSVKGTHYGQKAI LFLPLPVSSD (SEQ ID NO. 56) |
| | K PKLLYCSNGG HFLRILPDGT VDGTRDRSDQ HIQFQLSAES VGEVYIKSTE |
| | TGQYLAIDTD GLVYGSQTPN EECLFLERLE ENHYNTYISK KHGWFLG |
| | IKKNGSVKGTHYGQKAI LFLPLPVSSD (SEQ ID NO. 57) |
| | KLLYCSNGG HFLRILPDGT VDGTRDRSDQ HIQFQLSAES VGEVYIKSTE |
| | TGQYLAIDTD GLVYGSQTPN EECLFLERLE ENHYNTYISK KHGWFLG |
| | IKKNGSVKGTHYGQKAI LFLPLPVSSD (SEQ ID NO. 58) |
| | K PVLLYCSNGG HFLRILPDGT VDGTRDRSDQ HIQFQLSAES VGEVYIKSTE |
| | TGQYLAIDTD GLVYGSQTPN EECLFLERLE ENHYNTYISK KHGWFLG |
| | IKKNGSVKGTHYGQKAI LFLPLPVSSD (SEQ ID NO. 59) |
| | VLLYCSNGG HFLRILPDGT VDGTRDRSDQ HIQFQLSAES VGEVYIKSTE |
| | TGQYLAIDTD GLVYGSQTPN EECLFLERLE ENHYNTYISK KHGWFLG |
| | IKKNGSVKGTHYGQKAI LFLPLPVSSD (SEQ ID NO. 60) |
| | K PKLLYCSNGG HFLRILPDGT VDGTRDRSDQ HIQFQLSAES VGEVYIKSTE |
| | TGQYLAIDTD GLVYGSQTPN EECLFLERLE ENHYVTYISK KHGWFLG |
| | IKKNGSVKGTHYGQKAI LFLPLPVSSD (SEQ ID NO. 61) |
| | KLLYCSNGG HFLRILPDGT VDGTRDRSDQ HIQFQLSAES VGEVYIKSTE |
| | TGQYLAIDTD GLVYGSQTPN EECLFLERLE ENHYVTYISK KHGWFLG |
| | IKKNGSVKGTHYGQKAI LFLPLPVSSD (SEQ ID NO. 62) |
| | V PKLLYCSNGG HFLRILPDGT VDGTRDRSDQ HIQFQLSAES VGEVYIKSTE |
| | TGQYLAIDTD GLVYGSQTPN EECLFLERLE ENHYVTYISK KHGWFLG |
| | IKKNGSVKGTHYGQKAI LFLPLPVSSD (SEQ ID NO. 63) |
| | VLLYCSNGG HFLRILPDGT VDGTRDRSDQ HIQFQLSAES VGEVYIKSTE |
| | TGQYLAMTD GLVYGSQTPN EECLFLERLE ENHYVTYISK KHGWEIG |
| | IKKNGSVKGTHYGQKAI LFLPLPVSSD (SEQ ID NO. 64) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCNRG PRTHYGQKAI LFLPLPVSSD (SEQ ID NO. 65) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCERG PRTHYGQKAI LFLPLPVSSD (SEQ ID NO. 66) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSVKRG PRTHYGQKAI LFLPLPVSSD (SEQ ID NO. 67) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSVNRG PRTHYGQKAI LFLPLPVSSD (SEQ ID NO. 68) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSVERG PRTHYGQKAI LFLPLPVSSD (SEQ ID NO. 69) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 70) |
| | VLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 71) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 72) |
| | VLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 73) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 74) |
| | KLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 75) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCNRG PRTHYGQKAI LFLPLPVSSD (SEQ ID NO. 76) |
| | VLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSCERG PRTHYGQKAI LFLPLPVSSD (SEQ ID NO. 77) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCNRG PRTHYGQKAI LFLPLPVSSD (SEQ ID NO. 78) |
| | KLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCERG PRTHYGQKAI LFLPLPVSSD (SEQ ID NO. 79) |
| | FNLPPGNYTT PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO. 80) |
| | FNLPPGNYTT PKLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO. 81) |
| | FNLPPGNYTT PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCNRG PRTHYGQKAI LFLPLPVSSD (SEQ ID NO. 82) |
| | FNLPPGDQDQ NQLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO. 83) |
| | K PKLLYCSNGG HFLRILPDGT VDGTRDRSDP HIQLQLIAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 84) |
| | KLLYCSNGG HFLRILPDGT VDGTRDRSDP HIQLQLIAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO. 85) |
| | FNLPPGNYKK PVLLYCSNGG HFLRILPDGT VDGTRDRSDP HIQLQLIAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO. 86) |
| | K PVLLYCSNGG HFLRILPDGT VDGTRDRSDP HIQLQLIAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 87) |
| | VLLYCSNGG HFLRILPDGT VDGTRDRSDP HIQLQLIAES VGEVYIKSTE |
| | TGQYLAMDTD GLLYGSQTPN EECLFLERLE ENHYVTYISK KHAEKNWFVG |
| | LKKNGSCKRG PRTHYGQKAI LFLPLPVSSD (SEQ ID NO. 88) |
| | K PKLLYCSNGG HFLRILPDGT VDGTRDRSDP HIQLQLIAES VGEVYIKSTE |
| | TGQYLAMDTD GLLYGSQTPN EECLFLERLE ENGYNTYISK KHAEKNWFVG |
| | LKKNGSCKRG PRTHYGQKAI LFLPLPVSSD (SEQ ID NO. 89) |
| | KLLYCSNGG HFLRILPDGT VDGTRDRSDP HIQLQLIAES VGEVYIKSTE |
| | TGQYLAMDTD GLLYGSQTPN EECLFLERLE ENGYNTYISK KHAEKNWFVG |
| | LKKNGSCKRG PRTHYGQKAI LFLPLPVSSD (SEQ ID NO. 90) |
| | FNLPPGNYKK PVLLYCSNGG HFLRILPDGT VDGTRDRSDP HIQLQLIAES |
| | VGEVYIKSTE TGQYLAMDTD GLLYGSQTPN EECLFLERLE ENGYVTYISK |
| | KHAEKNWFVG LKKNGSCKRG PRTHYGQKAI LFLPLPVSSD (SEQ ID NO. 91) |
| | K PVLLYCSNGG HFLRILPDGT VDGTRDRSDP HIQLQLIAES VGEVYIKSTE |
| | TGQYLAMDTD GLLYGSQTPN EECLFLERLE ENGYVTYISK KHAEKNWFVG |
| | LKKNGSCKRG PRTHYGQKAI LFLPLPVSSD (SEQ ID NO. 92) |
| | VLLYCSNGG HFLRILPDGT VDGTRDRSDP HIQLQLIAES VGEVYIKSTE |
| | TGQYLAMDTDGLLYGSQTPN EECLFLERLE ENGYVTYISK KHAEKNWFVG |
| | LKKNGSCKRG PRTHYGQKAI LFLPLPVSSD (SEQ ID NO. 93) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSPVRG PRTHYGQKAI LFLPLPVSSD (SEQ ID NO. 94) |
| | KLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSPVRG PRTHYGQKAI LFLPLPVSSD (SEQ ID NO. 95) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSPVRG PRTHYGQKAI LFLPLPVSSD (SEQ ID NO. 96) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO. 97) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LDQNGSCVRG PRTHYGQKAI LFLPLPVSSD (SEQ ID NO. 98) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LDQNGSVVRG PRTHYGQKAI LFLPLPVSSD (SEQ ID NO. 99) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LDQNGSCVRG PRTHYGQKAI LFLPLPVSSD (SEQ ID NO. 100) |
| | KLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LDQNGSCVRG PRTHYGQKAI LFLPLPVSSD (SEQ ID NO. 101) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LDQNGSVVRG PRTHYGQKAI LFLPLPVSSD (SEQ ID NO. 102) |
| | KLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LDQNGSVVRG PRTHYGQKAI LFLPLPVSSD (SEQ ID NO. 103) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LDQNGSCVRG PRTHYGQKAI LFLPLPVSSD (SEQ ID NO. 104) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LDQNGSVVRG PRTHYGQKAI LFLPLPVSSD (SEQ ID NO. 105) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LDQNGSVVRG PRTHYGQKAI LFLPLPVSSD (SEQ ID NO. 106) |
| | FNLPPGNYKK PVLLYCSNGG HFLRILPDGT VDGTEDRSDQ HIQLQLSAES |
| | VGEVYIKSTE TGQYLAMDTD GLLYGSQTPN EECLFLERLE ENHYVTYISK |
| | KHAEKNWFVG LKKNGSVKRG PRTHYGQKAI LFLPLPVSSD (SEQ ID NO. 107) |
| | K PKLLYCSNGG HFLRILPDGT VDGTEDRSDQ HIQLQLSAES VGEVYIKSTE |
| | TGQYLAMDTD GLLYGSQTPN EECLFLERLE ENHYNTYISK KHAEKNWFVG |
| | LKKNGSVKRG PRTHYGQKAI LFLPLPVSSD (SEQ ID NO. 108) |
| | K PVLLYCSNGG HFLRILPDGT VDGTEDRSDQ HIQLQLSAES VGEVYIKSTE |
| | TGQYLAMDTD GLLYGSQTPN EECLFLERLE ENHYVTYISK KHAEKNWFVG |
| | LDQNGSVVRG PRTHYGQKAI LFLPLPVSSD (SEQ ID NO. 109) |
| | FNLPPGNYKK PVLLYCSNGG HFLRILPDGT VDGTLDRSDQ HIQLQLSAES |
| | VGEVYIKSTE TGQYLAMDTD GLLYGSQTPN EECLFLERLE ENHYVTYISK |
| | KHAEKNWFVG LDQNGSVVRG PRTHYGQKAI LFLPLPVSSD (SEQ ID NO. 110) |
| | K PVLLYCSNGG HFLRILPDGT VDGTEDRSDQ HIQFQLSAES VGEVYIKSTE |
| | TGQYLAMDTD GLLYGSQTPN EEILFLERLE ENHYVTYISK KHAEKNWFVG |
| | LKKNGSVKRG PRTHYGQKAI LWLPLPVSSD (SEQ ID NO. 111) |
| | K PVLLYCSNGG HFLRILPDGT VDGTEDRSDQ HIQFQLSAES VGEVYIKSTE |
| | TGQYLAMDTD GLLYGSQTPN EEILFLERLE ENHYVTYISK KHAEKNWFVG |
| | LDQNGSVVRG PRTHYGQKAI LWLPLPVSSD (SEQ ID NO. 112) |
| | FNLPPGNYKK PKLLYCSNGG HFLRILPDGT VDGTVDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO. 113) |
| | FNLPPGNYKK PKLLYCSNGG HFLRILPDGT VDGTVDRSDQ HIQLQLSAES |
| | VGEVYIKSTE TGQYLAMDTD GLLYGSQTPN EECLFLERLE ENHYNTYISK |
| | KHAEKNWFVG LDQNGSVVRG PRTHYGQKAI LFLPLPVSSD (SEQ ID NO. 114) |
| | FNLPPGNYKK PVLLYCSNGG HFLRILPDGT VDGTVDRSDQ HIQLQLSAES |
| | VGEVYIKSTE TGQYLAMDTD GLLYGSQTPN EECLFLERLE ENHYVTYISK |
| | KHAEKNWFVG LKKNGSVKRG PRTHYGQKAI LFLPLPVSSD (SEQ ID NO. 115) |
| | K PKLLYCSNGG HFLRILPDGT VDGTVDRSDQ HIQLQLSAES VGEVYIKSTE |
| | TGQYLAMDTD GLLYGSQTPN EECLFLERLE ENHYNTYISK KHAEKNWFVG |
| | LKKNGSVKRG PRTHYGQKAI LFLPLPVSSD (SEQ ID NO. 116) |
| | K PVLLYCSNGG HFLRILPDGT VDGTVDRSDQ HIQLQLSAES VGEVYIKSTE |
| | TGQYLAMDTD GLLYGSQTPN EECLFLERLE ENHYVTYISK KHAEKNWFVG |
| | LDQNGSVVRG PRTHYGQKAI LFLPLPVSSD (SEQ ID NO. 117) |
| | FNLPPGNYKK PVLLYCSNGG HFLRILPDGT VDGTVDRSDQ HIQLQLSAES |
| | VGEVYIKSTE TGQYLAMDTD GLLYGSQTPN EECLFLERLE ENHYVTYISK |
| | KHAEKNWFVG LDQNGSVVRG PRTHYGQKAI LFLPLPVSSD (SEQ ID NO. 118) |
| | K PVLLYCSNGG HFLRILPDGT VDGTVDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 119) |
| | K PVLLYCSNGG HFLRILPDGT VDGTVDRSDQ HIQFQLSAES VGEVYIKSTE |
| | TGQYLAMDTD GLLYGSQTPN EETLFLERLE ENHYVTYISK KHAEKNWFVG |
| | LDQNGSVVRG PRTHYGQKAI LWLPLPVSSD (SEQ ID NO. 120) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LDQNGSVVVG PRTHYGQKAI LFLPLPVSSD (SEQ ID NO. 121) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO. 122) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLVRLE ENHYNTYISK |
| | KHAEKNWFVG LKKNGSVKRG PRTHYGQKAI LFLPLPVSSD (SEQ ID NO. 123) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLVRLE ENHVNTYISK |
| | KHAEKNWFVG LKKNGSVKRG PRTHYGQKAI LFLPLPVSSD (SEQ ID NO. 124) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVVTYISK |
| | KHAEKNWFVG LKKNGSVKRG PRTHYGQKAI LFLPLPVSSD (SEQ ID NO. 125) |
| | FNLPPGNYKK PVLLYCSNGG HFLRILPDGT VDGTRDRSDQ HIQLQVSAES |
| | VGEVYIKSTE TGQYLAMDTD GLLYGSQTPN EECLFLVRLE ENHYVTYISK |
| | KHAEKNWFVG LKKNGSVKRG PRTHYGQKAI LFLVLPVSSD (SEQ ID NO. 126) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVVRG PRTHYGQKAI LFLPLPVSSD (SEQ ID NO. 127) |
| | K PVLLYCSNGG HFLRILPDGT VDGTRDRSDQ HIQLQLSAES VGEVYIKSTE |
| | TGQYLAMDTDGLLYGSQTPN EETLFLERLE ENHYVTYISK KHAEKNWFVG |
| | LKKNGSVKRG PRTHYGQKAI LFLPLPVSSD (SEQ ID NO. 128) |
| | K PVLLYTSNGG HFLRILPDGT VDGTRDRSDQ HIQLQLSAES VGEVYIKSTE |
| | TGQYLAMDTDGLLYGSQTPN EESLFLERLE ENHYVTYISK KHAEKNWFVG |
| | LKKNGSAKRG PRTHYGQKAI LFLPLPVSSD (SEQ ID NO. 129) |
| | K PKLLYCSNGG YFLRILPDGT VDGTRDRSDQ HIQFQLSAES VGEVYIKSTE |
| | TGQYLAMDTD GLLYGSQTPN EECLFLERLE ENHYNTYISK KYAEKNWYVG |
| | LKKNGSVKRG PRTHYGQKAI LFLPLPVSSD (SEQ ID NO. 130) |
| | K PVLLYCSNGG YFLRILPDGT VDGTRDRSDQ HIQFQLSAES VGEVYIKSTE |
| | TGQYLAMDTD GLLYGSQTPN EECLFLERLE ENHYVTYISK KYAEKNWYVG |
| | LKKNGSVKRG PRTHYGQKAI LFLPLPVSSD (SEQ ID NO. 131) |
| | FNLPPGNYKK PVLLYCSNGG YFLRILPDGT VDGTRDRSDQ HIQFQLSAES |
| | VGEVYIKSTE TGQYLAMDTD GLLYGSQTPN EECLFLERLE ENHYVTYISK |
| | KYAEKNWYVG LKKNGSVKRG PRTHYGQKAI LFLPLPVSSD (SEQ ID NO. 132) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO. 133) |
| | FNLPPGNYKK PKLLYCSNGG HFLRILPDGT VDGTRDRSDQ HIQLQLSAES |
| | VGEVYIKSTE TGQYLAMDTDGLLYGSQTPN EECLFLERLE ENHYNTYISK |
| | KHAEKNWFVG LDQNGSCVRG PRTHYGQKAI LFLPLPVSSD (SEQ ID NO. 134) |
| | FNLPPGNYKK PVLLYCSNGG HFLRILPDGT VDGTRDRSDP HIQLQLIAES |
| | VGEVYIKSTE TGQYLAMDTD GLLYGSQTPN EECLFLERLE ENGYVTYISK |
| | KHAEKNWFVG LKKNGSCKRG PRTHYGQKAI LFLPLPVSSD (SEQ ID NO. 135) |
| | K PVLLYCSNGG HFLRILPDGT VDGTRDRSDP HIQLQLIAES VGEVYIKSTE |
| | TGQYLAMDTD GLLYGSQTPN EECLFLERLE ENGYVTYISK KHAEKNWFVG |
| | LKKNGSCKRG PRTHYGQKAI LFLPLPVSSD (SEQ ID NO. 136) |
| | FNLPPGNYKK PVLLYCSNGG HFLRILPDGT VDGTRDRSDQ HIQFQLSAES |
| | VGEVYIKSTE TGQYLAMDTD GLLYGSQTPN EETLFLERLE ENHYVTYISK |
| | KHAEKNWFVG LKKNGSVKRG PRTHYGQKAI LWLPLPVSSD (SEQ ID NO. 137) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO. 138) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LDQNGSVVRG PRTHYGQKAI LFLPLPVSSD (SEQ ID NO. 139) |
| | K PVLLYCSNGG HFLRILPDGT VDGTRDRSDQ HIQFQLSAES VGEVYIKSTE |
| | TGQYLAMDTD GLLYGSQTPN EETLFLERLE ENHYVTYISK KHAEKNWFVG |
| | LKKNGSVKRG PRTHYGQKAI LWLPLPVSSD (SEQ ID NO. 140) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO. 141) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO. 142) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLVRLE ENHYNTYISK |
| | KHAEKNWFVG LKKNGSVKRG PRTHYGQKAI LFLPLPVSSD (SEQ ID NO. 143) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LDQNGSVVVG PRTHYGQKAI LFLPLPVSSD (SEQ ID NO. 144) |
| | FNLPPGNYKK PVLLYCSNGG HFLRILPDGT VDGTEDRSDQ HIQLQLSAES |
| | VGEVYIKSTE TGQYLAMDTD FNLPPGNYKK PVLLYCSNGG HFLRILPDGT |
| | VDGTEDRSDQ HIQLQLSAES VGEVYIKSTE TGQYLAMDTD (SEQ ID NO. 145) |
| | K PVLLYCSNGG HFLRILPDGT VDGTEDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO. 146) |
| | FNLPPGNYKK PKLLYCSNGG YFLRILPDGT VDGTRDRSDQ HIQFQLSAES |
| | VGEVYIKSTE TGQYLAMDTD GLLYGSQTPN EECLFLERLE ENHYNTYISK |
| | KYAEKNWYVG LKKNGSCKRG PRTHYGQKAI LFLPLPVSSD (SEQ ID NO. 147) |
| | K PKLLYCSNGG YFLRILPDGT VDGTRDRSDQ HIQFQLSAES VGEVYIKSTE |
| | TGQYLAMDTD HLHF GLLYGSQTPN EECLFLERLE ENHYNTYISK |
| | KYAEKNWYVG LKKNGSCKRG PRTHYGQKAI LFLPLPVSSD (SEQ ID NO. 148) |
| | FNLPPGNYKK PVLLYCSNGG YFLRILPDGT VDGTRDRSDQ HIQFQLSAES |
| | VGEVYIKSTE TGQYLAMDTD GLLYGSQTPN EECLFLERLE ENHYVTYISK |
| | KYAEKNWYVGLKKNGSCKRG PRTHYGQKAI LFLPLPVSSD (SEQ ID NO: 149) |
| | K PVLLYCSNGG YFLRILPDGT VDGTRDRSDQ HIQFQLSAES VGEVYIKSTE |
| | TGQYLAMDTD GLLYGSQTPN EECLFLERLE ENHYVTYISK KYAEKNWYVG |
| | LKKNGSVKRG PRTHYGQKAI LFLPLPVSSD (SEQ ID NO: 150) |
| | FNLPPGNYKK PKLLYCSNGG YFLRILPDGT VDGTRDRSDQ HIQFQLSAES |
| | VGEVYIKSTE TGQYLAMDTD GLLYGSQTPN EECLFLERLE ENHYNTYISK |
| | KYAEKNWYVG LKKNGSVKRG PRTHYGQKAI LFLPLPVSSD (SEQ ID NO: 151) |
| | FNLPPGNYKK PKLLYCSNGG YFLRILPDGT VDGTRDRSDQ HIQFQLSAES |
| | VGEVYIKSTE TGQYLCMDTD GLLYGSQTPN EECLFLERLE ENHYNTYISK |
| | KYAEKNWYVG LKKNGSVKRG PRTHYGQKAI LFLPLPVSSD (SEQ ID NO: 152) |
| | FNLPPGNYKK PKLLYCSNGG YFLRILPDGT VDGTEDRSDQ HIQFQLSAES |
| | VGEVYIKSTE TGQYLAMDTD GLLYGSQTPN EECLFLERLE ENHYNTYISK |
| | KYAEKNWYVG LKKNGSVKRG PRTHYGQKAI LFLPLPVSSD (SEQ ID NO: 153) |
| | FNLPPGNYKK PVLLYCSNGG YFLRILPDGT VDGTRDRSDQ HIQFQLSAES |
| | VGEVYIKSTE TGQYLAMDTD GLLYGSQTPN EECLFLERLE ENHVNTYISK |
| | KYAEKNWYVG LKKNGSVKRG PRTHYGQKAI LFLPLPVSSD (SEQ ID NO: 154) |
| | FNLPPGNYKK PKLLYCSRGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 155) |
| | -K PKLLYCSRGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 156) |
| | FNLPPGNYKKPVLLYCSRGGHFLRILPDGTVDGTRDRSDQHIQLQLSAESVGEVYIK |
| | STETGQYLAMDTDGLLYGSQTPNEECLFLERLEENHYVTYISKKHAEKNWFVG |
| | LKKNGSCKRG PRTHYGQKAI LFLPLPVSSD (SEQ ID NO: 157) |
| | K PVLLYCSRGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 158) |
| | FNLPPGNYKK PKLLYCSRGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 159) |
| | FNLPPGNYKK PKLLYCSRGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 160) |
| | FNLPPGNYKK PKLLYCSRGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 161) |
| | FNLPPGNYKK PVLLYCSRGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 162) |
| | FNLPPGNYKK PKLLYCS&GG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 163) |
| | K PKLLYCSKGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 164) |
| | FNLPPGNYKK PVLLYCSKGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 165) |
| | K PVLLYCSKGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 166) |
| | FNLPPGNYKK PKLLYCSKGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 167) |
| | FNLPPGNYKK PKLLYCSKGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 168) |
| | FNLPPGNYKK PKLLYCSKGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 169) |
| | FNLPPGNYKK PVLLYCSKGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 170) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKRGSCKRG PRTHYGQKAI LFLPLPVSSD (SEQ ID NO: 171) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKHGSCKRG PRTHYGQKAI LFLPLPVSSD (SEQ ID NO: 172) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKRGSCKRG PRTHYGQKAI LFLPLPVSSD (SEQ ID NO: 173) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKRGSVKRG PRTHYGQKAI LFLPLPVSSD (SEQ ID NO: 174) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKRGSVKRG PRTHYGQKAI LFLPLPVSSD (SEQ ID NO: 175) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKRGSCKRG PRTHYGQKAI LFLPLPVSSD (SEQ ID NO: 176) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKRGSVKRG PRTHYGQKAI LFLPLPVSSD (SEQ ID NO: 177) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKRGSVKRG PRTHYGQKAI LFLPLPVSSD (SEQ ID NO: 178) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKKGSCKRG PRTHYGQKAI LFLPLPVSSD (SEQ ID NO: 179) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKKGSCKRG PRTHYGQKAI LFLPLPVSSD (SEQ ID NO: 180) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKKGSCKRG PRTHYGQKAI LFLPLPVSSD (SEQ ID NO: 181) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKKGSVKRG PRTHYGQKAI LFLPLPVSSD (SEQ ID NO: 182) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKKGSVKRG PRTHYGQKAI LFLPLPVSSD (SEQ ID NO: 183) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKKGSCKRG PRTHYGQKAI LFLPLPVSSD (SEQ ID NO: 184) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKKGSVKRG PRTHYGQKAI LFLPLPVSSD (SEQ ID NO: 185) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKKGSVKRG PRTHYGQKAI LFLPLPVSSD (SEQ ID NO: 186) |
| | FNLPPGNYKK PKLLYCRNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 187) |
| | K PKLLYCRNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 188) |
| | FNLPPGNYKK PVLLYCRNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 189) |
| | K PVLLYCRNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 190) |
| | FNLPPGNYKK PKLLYCRNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 191) |
| | FNLPPGNYKK PKLLYCRNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 192) |
| | FNLPPGNYKK PKLLYCRNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 193) |
| | FNLPPGNYKK PVLLYCRNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 194) |
| | FNLPPGNYKK PKLLYCKNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 195) |
| | K PKLLYCKNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 196) |
| | FNLPPGNYKK PVLLYCKNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 197) |
| | K PVLLYCKNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 198) |
| | FNLPPGNYKK PKLLYCKNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 199) |
| | FNLPPGNYKK PKLLYCKNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 200) |
| | FNLPPGNYKK PKLLYCKNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 201) |
| | FNLPPGNYKK PVLLYCKNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 202) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGRKAI LFLPLPVSSD (SEQ ID NO: 203) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGRKAI LFLPLPVSSD (SEQ ID NO: 204) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGRKAI LFLPLPVSSD (SEQ ID NO: 205) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGRKAI LFLPLPVSSD (SEQ ID NO: 206) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGRKAI LFLPLPVSSD (SEQ ID NO: 207) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSCKRG PRTHYGRKAI LFLPLPVSSD (SEQ ID NO: 208) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGRKAI LFLPLPVSSD (SEQ ID NO: 209) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGRKAI LFLPLPVSSD (SEQ ID NO: 210) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGKKAI LFLPLPVSSD (SEQ ID NO: 211) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGKKAI LFLPLPVSSD (SEQ ID NO: 212) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGKKAI LFLPLPVSSD (SEQ ID NO: 213) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGKKAI LFLPLPVSSD (SEQ ID NO: 214) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGKKAI LFLPLPVSSD (SEQ ID NO: 215) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSCKRG PRTHYGKKAI LFLPLPVSSD (SEQ ID NO: 216) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGKKAI LFLPLPVSSD (SEQ ID NO: 217) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGKKAI LFLPLPVSSD (SEQ ID NO: 218) |
| | FNLPPGNYKK PKLLYCSNGG HFLRILPDGT VDGTRDRSDQ HIQLQLSADS |
| | VGEVYIKSTE TGQYLAMDTDGLLYGSQTPN EECLFLERLE ENHYNTYISK |
| | KHAEKNWFVG LKKNGSCKRG PRTHYGQKAI LFLPLPVSSD (SEQ ID NO: 219) |
| | K PKLLYCSNGG HFLRILPDGT VDGTRDRSDQ HIQLQLSADS VGEVYIKSTE |
| | TGQYLAMDTDGLLYGSQTPN EECLFLERLE ENHYNTYISK KHAEKNWFVG |
| | LKKNGSCKRG PRTHYGQKAI LFLPLPVSSD (SEQ ID NO: 220) |
| | FNLPPGNYKK PVLLYCSNGG HFLRILPDGT VDGTRDRSDQ HIQLQLSADS |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 221) |
| | K PVLLYCSNGG HFLRILPDGT VDGTRDRSDQ HIQLQLSADS VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 222) |
| | FNLPPGNYKK PKLLYCSNGG HFLRILPDGT VDGTRDRSDQ HIQLQLSADS |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 223) |
| | FNLPPGNYKK PKLLYCSNGG HFLRILPDGT VDGTRDRSDQ HIQLQLSADS |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 224) |
| | FNLPPGNYKK PKLLYCSNGG HFLRILPDGT VDGTEDRSDQ HIQLQLSADS |
| | VGEVYIKSTE TGQYLAMDTDGLLYGSQTPN EECLFLERLE ENHYNTYISK |
| | KHAEKNWFVG LKKNGSVKRG PRTHYGQKAI LFLPLPVSSD (SEQ ID NO: 225) |
| | FNLPPGNYKK PVLLYCSNGG HFLRILPDGT VDGTRDRSDQ HIQLQLSADS |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 226) |
| | FNLPPGNYKK PKLLYCSNGG HFLRILPDGT VDGTRDRSDQ HIQLQLSAKS |
| | VGEVYIKSTE TGQYLAMDTDGLLYGSQTPN EECLFLERLE ENHYNTYISK |
| | KHAEKNVVFVG LKKNGSCKRG PRTHYGQKAI LFLPLPVSSD (SEQ ID NO: 227) |
| | K PKLLYCSNGG HFLRILPDGT VDGTRDRSDQ HIQLQLSAKS VGEVYIKSTE |
| | TGQYLAMDTD GLLYGSQTPN EECLFLERLE ENHYNTYISK KHAEKNVVFVG |
| | LKKNGSCKRG PRTHYGQKAI LFLPLPVSSD (SEQ ID NO: 228) |
| | FNLPPGNYKK PVLLYCSNGG HFLRILPDGT VDGTRDRSDQ HIQLQLSAKS |
| | VGEVYIKSTE TGQYLAMDTD GLLYGSQTPN EECLFLERLE ENHYVTYISK |
| | KHAEKNVVFVG LKKNGSCKRG PRTHYGQKAI LFLPLPVSSD (SEQ ID NO: 229) |
| | K PVLLYCSNGG HFLRILPDGT VDGTRDRSDQ HIQLQLSAKS VGEVYIKSTE |
| | TGQYLAMDTD GLLYGSQTPN EECLFLERLE ENHYVTYISK KHAEKNVVFVG |
| | LKKNGSVKRG PRTHYGQKAI LFLPLPVSSD (SEQ ID NO: 230) |
| | FNLPPGNYKK PKLLYCSNGG HFLRILPDGT VDGTRDRSDQ HIQLQLSAKS |
| | VGEVYIKSTE TGQYLAMDTD GLLYGSQTPN EECLFLERLE ENHYNTYISK |
| | KHAEKNVVFVG LKKNGSVKRG PRTHYGQKAI LFLPLPVSSD (SEQ ID NO: 231) |
| | FNLPPGNYKK PKLLYCSNGG HFLRILPDGT VDGTRDRSDQ HIQLQLSAKS |
| | VGEVYIKSTE TGQYLCMDTD GLLYGSQTPN EECLFLERLE ENHYNTYISK |
| | KHAEKNVVFVG LKKNGSVKRG PRTHYGQKAI LFLPLPVSSD (SEQ ID NO: 232) |
| | FNLPPGNYKK PKLLYCSNGG HFLRILPDGT VDGTEDRSDQ HIQLQLSAKS |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 233) |
| | FNLPPGNYKK PVLLYCSNGG HFLRILPDGT VDGTRDRSDQ HIQLQLSAKS |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 234) |
| | FNLPPGNYKK PKLLYCSNGG HFLRILPDGT VDGTRDRSDQ HIQLQLSAES |
| | VGEVFIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 235) |
| | K PKLLYCSNGG HFLRILPDGT VDGTRDRSDQ HIQLQLSAES VGEVFIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 236) |
| | FNLPPGNYKK PVLLYCSNGG HFLRILPDGT VDGTRDRSDQ HIQLQLSAES |
| | VGEVFIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 237) |
| | K PVLLYCSNGG HFLRILPDGT VDGTRDRSDQ HIQLQLSAES VGEVFIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 238) |
| | FNLPPGNYKK PKLLYCSNGG HFLRILPDGT VDGTRDRSDQ HIQLQLSAES |
| | VGEVFIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 239) |
| | FNLPPGNYKK PKLLYCSNGG HFLRILPDGT VDGTRDRSDQ HIQLQLSAES |
| | VGEVFIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 240) |
| | FNLPPGNYKK PKLLYCSNGG HFLRILPDGT VDGTEDRSDQ HIQLQLSAES |
| | VGEVFIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 241) |
| | FNLPPGNYKK PVLLYCSNGG HFLRILPDGT VDGTRDRSDQ HIQLQLSAES |
| | VGEVFIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 242) |
| | FNLPPGNYKK PKLLYCSNGG HFLRILPDGT VDGTRDRSDQ HIQLQLSAES |
| | VGEVVIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 243) |
| | K PKLLYCSNGG HFLRILPDGT VDGTRDRSDQ HIQLQLSAES VGEVVIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 244) |
| | FNLPPGNYKK PVLLYCSNGG HFLRILPDGT VDGTRDRSDQ HIQLQLSAES |
| | VGEVVIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 245) |
| | K PVLLYCSNGG HFLRILPDGT VDGTRDRSDQ HIQLQLSAES VGEVVIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 246) |
| | FNLPPGNYKK PKLLYCSNGG HFLRILPDGT VDGTRDRSDQ HIQLQLSAES |
| | VGEVVIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 247) |
| | FNLPPGNYKK PKLLYCSNGG HFLRILPDGT VDGTRDRSDQ HIQLQLSAES |
| | VGEVVIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 248) |
| | FNLPPGNYKK PKLLYCSNGG HFLRILPDGT VDGTEDRSDQ HIQLQLSAES |
| | VGEVVIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 249) |
| | FNLPPGNYKK PVLLYCSNGG HFLRILPDGT VDGTRDRSDQ HIQLQLSAES |
| | VGEVVIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 250) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLELLE ENHYNTYISK |
| | KHAEKNWFVG LKKNGSCKRG PRTHYGQKAI LFLPLPVSSD (SEQ ID NO: 251) |
| | K PKLLYCSNGG HFLRILPDGT VDGTRDRSDQ HIQLQLSAES VGEVYIKSTE |
| | TGQYLAMDTD GLLYGSQTPN EECLFLELLE ENHYNTYISK KHAEKNWFVG |
| | LKKNGSCKRG PRTHYGQKAI LFLPLPVSSD (SEQ ID NO: 252) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLELLE ENHYVTYISK |
| | KHAEKNWFVG LKKNGSCKRG PRTHYGQKAI LFLPLPVSSD (SEQ ID NO: 253) |
| | K PVLLYCSNGG HFLRILPDGT VDGTRDRSDQ HIQLQLSAES VGEVYIKSTE |
| | TGQYLAMDTD GLLYGSQTPN EECLFLELLE ENHYVTYISK KHAEKNWFVG |
| | LKKNGSVKRG PRTHYGQKAI LFLPLPVSSD (SEQ ID NO: 254) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLELLE ENHYNTYISK |
| | KHAEKNWFVG LKKNGSVKRG PRTHYGQKAI LFLPLPVSSD (SEQ ID NO: 255) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLELLE ENHYNTYISK |
| | KHAEKNWFVG LKKNGSVKRG PRTHYGQKAI LFLPLPVSSD (SEQ ID NO: 256) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLELLE ENHYNTYISK |
| | KHAEKNWFVG LKKNGSVKRG PRTHYGQKAI LFLPLPVSSD (SEQ ID NO: 257) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLELLE ENHVNTYISK |
| | KHAEKNWFVG LKKNGSVKRG PRTHYGQKAI LFLPLPVSSD (SEQ ID NO: 258) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLEYLE ENHYNTYISK |
| | KHAEKNWFVG LKKNGSCKRG PRTHYGQKAI LFLPLPVSSD (SEQ ID NO: 259) |
| | K PKLLYCSNGG HFLRILPDGT VDGTRDRSDQ HIQLQLSAES VGEVYIKSTE |
| | TGQYLAMDTD GLLYGSQTPN EECLFLEYLE ENHYNTYISK KHAEKNWFVG |
| | LKKNGSCKRG PRTHYGQKAI LFLPLPVSSD (SEQ ID NO: 260) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLEYLE ENHYVTYISK |
| | KHAEKNWFVG LKKNGSCKRG PRTHYGQKAI LFLPLPVSSD (SEQ ID NO: 261) |
| | K PVLLYCSNGG HFLRILPDGT VDGTRDRSDQ HIQLQLSAES VGEVYIKSTE |
| | TGQYLAMDTD GLLYGSQTPN EECLFLEYLE ENHYVTYISK KHAEKNWFVG |
| | LKKNGSVKRG PRTHYGQKAI LFLPLPVSSD (SEQ ID NO: 262) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLEYLE ENHYNTYISK |
| | KHAEKNWFVG LKKNGSVKRG PRTHYGQKAI LFLPLPVSSD (SEQ ID NO: 263) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLEYLE ENHYNTYISK |
| | KHAEKNWFVG LKKNGSVKRG PRTHYGQKAI LFLPLPVSSD (SEQ ID NO: 264) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLEYLE ENHYNTYISK |
| | KHAEKNWFVG LKKNGSVKRG PRTHYGQKAI LFLPLPVSSD (SEQ ID NO: 265) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLEYLE ENHVNTYISK |
| | KHAEKNWFVG LKKNGSVKRG PRTHYGQKAI LFLPLPVSSD (SEQ ID NO: 266) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLEDLE ENHYNTYISK |
| | KHAEKNWFVG LKKNGSCKRG PRTHYGQKAI LFLPLPVSSD (SEQ ID NO: 267) |
| | K PKLLYCSNGG HFLRILPDGT VDGTRDRSDQ HIQLQLSAES VGEVYIKSTE |
| | TGQYLAMDTD GLLYGSQTPN EECLFLEDLE ENHYNTYISK KHAEKNWFVG |
| | LKKNGSCKRG PRTHYGQKAI LFLPLPVSSD (SEQ ID NO: 268) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLEDLE ENHYVTYISK |
| | KHAEKNWFVG LKKNGSCKRG PRTHYGQKAI LFLPLPVSSD (SEQ ID NO: 269) |
| | K PVLLYCSNGG HFLRILPDGT VDGTRDRSDQ HIQLQLSAES VGEVYIKSTE |
| | TGQYLAMDTD GLLYGSQTPN EECLFLEDLE ENHYVTYISK KHAEKNWFVG |
| | LKKNGSVKRG PRTHYGQKAI LFLPLPVSSD (SEQ ID NO: 270) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLEDLE ENHYNTYISK |
| | KHAEKNWFVG LKKNGSVKRG PRTHYGQKAI LFLPLPVSSD (SEQ ID NO: 271) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLEDLE ENHYNTYISK |
| | KHAEKNWFVG LKKNGSVKRG PRTHYGQKAI LFLPLPVSSD (SEQ ID NO: 272) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLEDLE ENHYNTYISK |
| | KHAEKNWFVG LKKNGSVKRG PRTHYGQKAI LFLPLPVSSD (SEQ ID NO: 273) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLEDLE ENHVNTYISK |
| | KHAEKNWFVG LKKNGSVKRG PRTHYGQKAI LFLPLPVSSD (SEQ ID NO: 274) |
| | FNLPPGNYKK PKLLYCSNGG HFLRILPDGT VDGTRDRSDQ HIKLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 275) |
| | K PKLLYCSNGG HFLRILPDGT VDGTRDRSDQ HIKLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 276) |
| | FNLPPGNYKK PVLLYCSNGG HFLRILPDGT VDGTRDRSDQ HIKLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 277) |
| | K PVLLYCSNGG HFLRILPDGT VDGTRDRSDQ HIKLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 278) |
| | FNLPPGNYKK PKLLYCSNGG HFLRILPDGT VDGTRDRSDQ HIKLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 279) |
| | FNLPPGNYKK PKLLYCSNGG HFLRILPDGT VDGTRDRSDQ HIKLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 280) |
| | FNLPPGNYKK PKLLYCSNGG HFLRILPDGT VDGTEDRSDQ HIKLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 281) |
| | FNLPPGNYKK PVLLYCSNGG HFLRILPDGT VDGTRDRSDQ HIKLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 282) |
| | FNLPPGNYKK PKLLYCSNGG HFLRILPDGT VDGTRDRSDQ HIALQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 283) |
| | K PKLLYCSNGG HFLRILPDGT VDGTRDRSDQ HIALQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 284) |
| | FNLPPGNYKK PVLLYCSNGG HFLRILPDGT VDGTRDRSDQ HIALQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 285) |
| | K PVLLYCSNGG HFLRILPDGT VDGTRDRSDQ HIALQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 286) |
| | FNLPPGNYKK PKLLYCSNGG HFLRILPDGT VDGTRDRSDQ HIALQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 287) |
| | FNLPPGNYKK PKLLYCSNGG HFLRILPDGT VDGTRDRSDQ HIALQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 288) |
| | FNLPPGNYKK PKLLYCSNGG HFLRILPDGT VDGTEDRSDQ HIALQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 289) |
| | FNLPPGNYKK PVLLYCSNGG HFLRILPDGT VDGTRDRSDQ HIALQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 290) |
| | FNLPPGNYKK PKLLYCSNGG HFLRILPDGT VDGTRDRSDQ HIELQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 291) |
| | K PKLLYCSNGG HFLRILPDGT VDGTRDRSDQ HIELQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 292) |
| | FNLPPGNYKK PVLLYCSNGG HFLRILPDGT VDGTRDRSDQ HIELQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 293) |
| | K PVLLYCSNGG HFLRILPDGT VDGTRDRSDQ HIELQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 294) |
| | FNLPPGNYKK PKLLYCSNGG HFLRILPDGT VDGTRDRSDQ HIELQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 295) |
| | FNLPPGNYKK PKLLYCSNGG HFLRILPDGT VDGTRDRSDQ HIELQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 296) |
| | FNLPPGNYKK PKLLYCSNGG HFLRILPDGT VDGTEDRSDQ HIELQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 297) |
| | FNLPPGNYKK PVLLYCSNGG HFLRILPDGT VDGTRDRSDQ HIELQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 298) |
| | FNLPPGNYKK PKLLYCSNGG HFLRILPDGT VDGTRDRSDQ HIQLQLAAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 299) |
| | K PKLLYCSNGG HFLRILPDGT VDGTRDRSDQ HIQLQLAAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 300) |
| | FNLPPGNYKK PVLLYCSNGG HFLRILPDGT VDGTRDRSDQ HIQLQLAAES |
| | VGEVYIKSTE TGQYLAMDTDGLLYGSQTPN EECLFLERLE ENHYVTYISK |
| | KHAEKNWFVG LKKNGSCKRG PRTHYGQKAI LFLPLPVSSD (SEQ ID NO: 301) |
| | K PVLLYCSNGG HFLRILPDGT VDGTRDRSDQ HIQLQLAAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 302) |
| | FNLPPGNYKK PKLLYCSNGG HFLRILPDGT VDGTRDRSDQ HIQLQLAAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 303) |
| | FNLPPGNYKK PKLLYCSNGG HFLRILPDGT VDGTRDRSDQ HIQLQLAAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 304) |
| | FNLPPGNYKK PKLLYCSNGG HFLRILPDGT VDGTEDRSDQ HIQLQLAAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 305) |
| | FNLPPGNYKK PVLLYCSNGG HFLRILPDGT VDGTRDRSDQ HIQLQLAAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 306) |
| | FNLPPGNYKK PKLLYCSNGG HFLRILPDGT VDGTRDRSDQ HIQLQLVAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 307) |
| | K PKLLYCSNGG HFLRILPDGT VDGTRDRSDQ HIQLQLVAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 308) |
| | FNLPPGNYKK PVLLYCSNGG HFLRILPDGT VDGTRDRSDQ HIQLQLVAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 309) |
| | K PVLLYCSNGG HFLRILPDGT VDGTRDRSDQ HIQLQLVAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 310) |
| | FNLPPGNYKK PKLLYCSNGG HFLRILPDGT VDGTRDRSDQ HIQLQLVAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 311) |
| | FNLPPGNYKK PKLLYCSNGG HFLRILPDGT VDGTRDRSDQ HIQLQLVAES |
| | VGEVYIKSTE TGQYLCMDTDGLLYGSQTPN EECLFLERLE ENHYNTYISK |
| | KHAEKNWFVG LKKNGSVKRG PRTHYGQKAI LFLPLPVSSD (SEQ ID NO: 312) |
| | FNLPPGNYKK PKLLYCSNGG HFLRILPDGT VDGTEDRSDQ HIQLQLVAES |
| | VGEVYIKSTE TGQYLAMDTDGLLYGSQTPN EECLFLERLE ENHYNTYISK |
| | KHAEKNWFVG LKKNGSVKRG PRTHYGQKAI LFLPLPVSSD (SEQ ID NO: 313) |
| | FNLPPGNYKK PVLLYCSNGG HFLRILPDGT VDGTRDRSDQ HIQLQLVAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 314) |
| | FNLPPGNYKK PKLLFCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 315) |
| | K PKLLFCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 316) |
| | FNLPPGNYKK PVLLFCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 317) |
| | K PVLLFCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 318) |
| | FNLPPGNYKK PKLLFCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 319) |
| | FNLPPGNYKK PKLLFCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 320) |
| | FNLPPGNYKK PKLLFCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 321) |
| | FNLPPGNYKK PVLLFCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 322) |
| | FNLPPGNYKK PKLLACSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 323) |
| | K PKLLACSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 324) |
| | FNLPPGNYKK PVLLACSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNVVFVG LKKNGSCKRG PRTHYGQKAI LFLPLPVSSD (SEQ ID NO: 325) |
| | K PVLLACSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 326) |
| | FNLPPGNYKK PKLLACSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 327) |
| | FNLPPGNYKK PKLLACSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 328) |
| | FNLPPGNYKK PKLLACSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 329) |
| | FNLPPGNYKK PVLLACSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 330) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFVPLPVSSD (SEQ ID NO: 331) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFVPLPVSSD (SEQ ID NO: 332) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFVPLPVSSD (SEQ ID NO: 333) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFVPLPVSSD (SEQ ID NO: 334) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFVPLPVSSD (SEQ ID NO: 335) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFVPLPVSSD (SEQ ID NO: 336) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFVPLPVSSD (SEQ ID NO: 337) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFVPLPVSSD (SEQ ID NO: 338) |
| | FNLPPGNYKK PKLLYCSNGG HFLRILPDGT VDGTRDRSDQ HIQLQLSAES |
| | VGEVYIKSTE TGQYLAMDTDGLLYGSQTPN EECLFLERLE ENHYNTYISK |
| | KHAEKNWFVG LKKNGSCKRG PRTHYGQKAI LFAPLPVSSD (SEQ ID NO: 339) |
| | K PKLLYCSNGG HFLRILPDGT VDGTRDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFAPLPVSSD (SEQ ID NO: 340) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFAPLPVSSD (SEQ ID NO: 341) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFAPLPVSSD (SEQ ID NO: 342) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFAPLPVSSD (SEQ ID NO: 343) |
| | FNLPPGNYKK PKLLYCSNGG HFLRILPDGT VDGTRDRSDQ HIQLQLSAES |
| | VGEVYIKSTE TGQYLCMDTDGLLYGSQTPN EECLFLERLE ENHYNTYISK |
| | KHAEKNWFVG LKKNGSVKRG PRTHYGQKAI LFAPLPVSSD (SEQ ID NO: 344) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFAPLPVSSD (SEQ ID NO: 345) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFAPLPVSSD (SEQ ID NO: 346) |
| | FNLPPGNYKK PKLLYCSNGG HFLRILPDGT VDGTKDRSDQ HIQLQLSAES |
| | VGEVYIKSTE TGQYLAMDTDGLLYGSQTPN EECLFLERLE ENHYNTYISK |
| | KHAEKNWFVG LKKNGSCKRG PRTHYGQKAI LFLPLPVSSD (SEQ ID NO: 347) |
| | K PKLLYCSNGG HFLRILPDGT VDGTKDRSDQ HIQLQLSAES VGEVYIKSTE |
| | TGQYLAMDTD GLLYGSQTPN EECLFLERLE ENHYNTYISK KHAEKNWFVG |
| | LKKNGSCKRG PRTHYGQKAI LFLPLPVSSD (SEQ ID NO: 348) |
| | FNLPPGNYKK PVLLYCSNGG HFLRILPDGT VDGTKDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 349) |
| | K PVLLYCSNGG HFLRILPDGT VDGTKDRSDQ HIQLQLSAES VGEVYIKSTE |
| | TGQYLAMDTD GLLYGSQTPN EECLFLERLE ENHYVTYISK KHAEKNWFVG |
| | LKKNGSVKRG PRTHYGQKAI LFLPLPVSSD (SEQ ID NO: 350) |
| | FNLPPGNYKK PKLLYCSNGG HFLRILPDGT VDGTKDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 351) |
| | FNLPPGNYKK PKLLYCSNGG HFLRILPDGT VDGTKDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 352) |
| | FNLPPGNYKK PKLLYCSNGG HFLRILPDGT VDGTKDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 353) |
| | FNLPPGNYKK PVLLYCSNGG HFLRILPDGT VDGTKDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 354) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLQRLE ENHYNTYISK |
| | KHAEKNWFVG LKKNGSCKRG PRTHYGQKAI LFLPLPVSSD (SEQ ID NO: 355) |
| | K PKLLYCSNGG HFLRILPDGT VDGTRDRSDQ HIQLQLSAES VGEVYIKSTE |
| | TGQYLAMDTDGLLYGSQTPN EECLFLQRLE ENHYNTYISK KHAEKNWFVG |
| | LKKNGSCKRG PRTHYGQKAI LFLPLPVSSD (SEQ ID NO: 356) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLQRLE ENHYVTYISK |
| | KHAEKNWFVG LKKNGSCKRG PRTHYGQKAI LFLPLPVSSD (SEQ ID NO: 357) |
| | K PVLLYCSNGG HFLRILPDGT VDGTRDRSDQ HIQLQLSAES VGEVYIKSTE |
| | TGQYLAMDTD GLLYGSQTPN EECLFLQRLE ENHYVTYISK KHAEKNWFVG |
| | LKKNGSVKRG PRTHYGQKAI LFLPLPVSSD (SEQ ID NO: 358) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLQRLE ENHYNTYISK |
| | KHAEKNWFVG LKKNGSVKRG PRTHYGQKAI LFLPLPVSSD (SEQ ID NO: 359) |
| | FNLPPGNYKK PKLLYCSNGG HFLRILPDGT VDGTRDRSDQ HIQLQLSAES |
| | VGEVYIKSTE TGQYLCMDTD GLLYGSQTPN EECLFLQRLE ENHYNTYISK |
| | KHAEKNWFVG LKKNGSVKRG PRTHYGQKAI LFLPLPVSSD (SEQ ID NO: 360) |
| | FNLPPGNYKK PKLLYCSNGG HFLRILPDGT VDGTEDRSDQ HIQLQLSAES |
| | VGEVYIKSTE TGQYLAMDTDGLLYGSQTPN EECLFLQRLE ENHYNTYISK |
| | KHAEKNWFVG LKKNGSVKRG PRTHYGQKAI LFLPLPVSSD (SEQ ID NO: 361) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLQRLE ENHVNTYISK |
| | KHAEKNWFVG LKKNGSVKRG PRTHYGQKAI LFLPLPVSSD (SEQ ID NO: 362) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLDRLE ENHYNTYISK |
| | KHAEKNWFVG LKKNGSCKRG PRTHYGQKAI LFLPLPVSSD (SEQ ID NO: 363) |
| | K PKLLYCSNGG HFLRILPDGT VDGTRDRSDQ HIQLQLSAES VGEVYIKSTE |
| | TGQYLAMDTD GLLYGSQTPN EECLFLDRLE ENHYNTYISK KHAEKNWFVG |
| | LKKNGSCKRG PRTHYGQKAI LFLPLPVSSD (SEQ ID NO: 364) |
| | FNLPPGNYKK PVLLYCSNGG HFLRILPDGT VDGTRDRSDQ HIQLQLSAES |
| | VGEVYIKSTE TGQYLAMDTDGLLYGSQTPN EECLFLDRLE ENHYVTYISK |
| | KHAEKNWFVG LKKNGSCKRG PRTHYGQKAI LFLPLPVSSD (SEQ ID NO: 365) |
| | K PVLLYCSNGG HFLRILPDGT VDGTRDRSDQ HIQLQLSAES VGEVYIKSTE |
| | TGQYLAMDTDGLLYGSQTPN EECLFLDRLE ENHYVTYISK KHAEKNWFVG |
| | LKKNGSVKRG PRTHYGQKAI LFLPLPVSSD (SEQ ID NO: 366) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLDRLE ENHYNTYISK |
| | KHAEKNWFVG LKKNGSVKRG PRTHYGQKAI LFLPLPVSSD (SEQ ID NO: 367) |
| | FNLPPGNYKK PKLLYCSNGG HFLRILPDGT VDGTRDRSDQ HIQLQLSAES |
| | VGEVYIKSTE TGQYLCMDTDGLLYGSQTPN EECLFLDRLE ENHYNTYISK |
| | KHAEKNWFVG LKKNGSVKRG PRTHYGQKAI LFLPLPVSSD (SEQ ID NO: 368) |
| | FNLPPGNYKK PKLLYCSNGG HFLRILPDGT VDGTEDRSDQ HIQLQLSAES |
| | VGEVYIKSTE TGQYLAMDTD GLLYGSQTPN EECLFLDRLE ENHYNTYISK |
| | KHAEKNWFVG LKKNGSVKRG PRTHYGQKAI LFLPLPVSSD (SEQ ID NO: 369) |
| | FNLPPGNYKK PVLLYCSNGG HFLRILPDGT VDGTRDRSDQ HIQLQLSAES |
| | VGEVYIKSTE TGQYLAMDTD GLLYGSQTPN EECLFLDRLE ENHVNTYISK |
| | KHAEKNWFVG LKKNGSVKRG PRTHYGQKAI LFLPLPVSSD (SEQ ID NO: 370) |
| | FNLPPGNFKK PKLLYCSNG HFLRILPDGT VDGTRDRSDQ HIQLQLSAES |
| | VGEVYIKSTE TGQYLAMDTDGLLYGSQTPN EECLFLERLE ENHYNTYISK |
| | KHAEKNWFVG LKKNGSCKRG PRTHYGQKAI LFLPLPVSSD (SEQ ID NO: 371) |
| | FNLPPGNFKK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 372) |
| | FNLPPGNFKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 373) |
| | FNLPPGNFKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 374) |
| | FNLPPGNFKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 375) |
| | FNLPPGNFKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 376) |
| | FNLPPGNVKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 377) |
| | FNLPPGNVKK PVLLYCSNGG HFLRILPDGT VDGTRDRSDQ HIQLQLSAES |
| | VGEVYIKSTE TGQYLAMDTDGLLYGSQTPN EECLFLERLE ENHYVTYISK |
| | KHAEKNWFVG LKKNGSCKRG PRTHYGQKAI LFLPLPVSSD (SEQ ID NO: 378) |
| | FNLPPGNVKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 379) |
| | FNLPPGNVKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 380) |
| | FNLPPGNVKK PKLLYCSNGG HFLRILPDGT VDGTEDRSDQ HIQLQLSAES |
| | VGEVYIKSTE TGQYLAMDTDGLLYGSQTPN EECLFLERLE ENHYNTYISK |
| | KHAEKNWFVG LKKNGSVKRG PRTHYGQKAI LFLPLPVSSD (SEQ ID NO: 381) |
| | FNLPPGNVKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 382) |
| | FNLPPGNAKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 383) |
| | FNLPPGNAKK PVLLYCSNGG HFLRILPDGT VDGTRDRSDQ HIQLQLSAES |
| | VGEVYIKSTE TGQYLAMDTDGLLYGSQTPN EECLFLERLE ENHYVTYISK |
| | KHAEKNWFVG LKKNGSCKRG PRTHYGQKAI LFLPLPVSSD (SEQ ID NO: 384) |
| | FNLPPGNAKK PKLLYCSNGG HFLRILPDGT VDGTRDRSDQ HIQLQLSAES |
| | VGEVYIKSTE TGQYLAMDTDGLLYGSQTPN EECLFLERLE ENHYNTYISK |
| | KHAEKNWFVG LKKNGSVKRG PRTHYGQKAI LFLPLPVSSD (SEQ ID NO: 385) |
| | FNLPPGNAKK PKLLYCSNGG HFLRILPDGT VDGTRDRSDQ HIQLQLSAES |
| | VGEVYIKSTE TGQYLCMDTD GLLYGSQTPN EECLFLERLE ENHYNTYISK |
| | KHAEKNWFVG LKKNGSVKRG PRTHYGQKAI LFLPLPVSSD (SEQ ID NO: 386) |
| | FNLPPGNAKK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 387) |
| | FNLPPGNAKK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 388) |
| | FNLPPGNYRK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 389) |
| | FNLPPGNYRK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 390) |
| | FNLPPGNYRK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 391) |
| | FNLPPGNYRK PKLLYCSNGG HFLRILPDGT VDGTRDRSDQ HIQLQLSAES |
| | VGEVYIKSTE TGQYLCMDTDGLLYGSQTPN EECLFLERLE ENHYNTYISK |
| | KHAEKNWFVG LKKNGSVKRG PRTHYGQKAI LFLPLPVSSD (SEQ ID NO: 392) |
| | FNLPPGNYRK PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 393) |
| | FNLPPGNYRK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 394) |
| | FNLPPGNYAK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSCKRG PRTHYGQKAI LFLPLPVSSD (SEQ ID NO: 395) |
| | FNLPPGNYAK PVLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGSCKRG PRTHYGQKAI LFLPLPVSSD (SEQ ID NO: 396) |
| | FNLPPGNYAK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGSVKRG PRTHYGQKAI LFLPLPVSSD (SEQ ID NO: 397) |
| | FNLPPGNYAK PKLLYCSNGG HFLRILPDGT VDGTRDRSDQ HIQLQLSAES |
| | VGEVYIKSTE TGQYLCMDTDGLLYGSQTPN EECLFLERLE ENHYNTYISK |
| | KHAEKNWFVG LKKNGSVKRG PRTHYGQKAI LFLPLPVSSD (SEQ ID NO: 398) |
| | FNLPPGNYAK PKLLYCSNGG HFLRILPDGT VDGTEDRSDQ HIQLQLSAES |
| | VGEVYIKSTE TGQYLAMDTDGLLYGSQTPN EECLFLERLE ENHYNTYISK |
| | KHAEKNWFVG LKKNGSVKRG PRTHYGQKAI LFLPLPVSSD (SEQ ID NO: 399) |
| | FNLPPGNYAK PVLLYCSNGG HFLRILPDGT VDGTRDRSDQ HIQLQLSAES |
| | VGEVYIKSTE TGQYLAMDTD GLLYGSQTPN EECLFLERLE ENHVNTYISK |
| | KHAEKNWFVG LKKNGSVKRG PRTHYGQKAI LFLPLPVSSD (SEQ ID NO: 400) |
| | K PKLLYCSNGG HFLRILPDGT VDGTRDRSDQ HIQLQLSAES |
| | VGEVYIKSTE TGQYLCMDTDGLLYGSQTPN EECLFLERLE ENHYVTYISK |
| | KHAEKNWFVG LKKNGRVKRG PRTHYGQKAI LFLPLPVSSD (SEQ ID NO: 401) |
| | K PVLLYCSNGG YFLRILPDGT VDGTRDRSDQ HIQFQLSAES |
| | VGEVYIKSTE TGQYLCMDTDGLLYGSQTPN EECLFLERLE ENHYVTYISK |
| | KYAEKNWYVG LKKNGSVKRG PRTHYGQKAI LFLPLPVSSD (SEQ ID NO: 402) |
| | FNLPPGNYKK PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGRCKRG PRTHYGQKAI LFLPLPVSSD (SEQ ID NO: 403) |
| | Salk_050 FNLPPGNYKK PVLLYCSNGG HFLRILPDGT VDGTRDRSDQ |
| | HIQLQLSAES VGEVYIKSTE TGQYLAMDTD GLLYGSQTPN EECLFLERLE |
| | ENHYVTYISK KHAEKNWFVG LKKNGRVKRG PRTHYGQKAI LFLPLPVSSD (SEQ |
| | ID NO: 404) |
| | Salk_051 K PKLLYCSNGG HFLRILPDGT VDGTRDRSDQ HIQLQLSAES |
| | VGEVYIKSTE TGQYLAMDTD GLLYGSQTPN EECLFLERLE ENHYNTYISK |
| | KHAEKNWFVG LKKNGRVKRG PRTHYGQKAI LFLPLPVSSD (SEQ ID NO: 405) |
| | Salk_052 K PKLLYCSNGG HFLRILPDGT VDGTRDRSDQ HIQLQLSAES |
| | VGEVYIKSTE TGQYLAMDTD GLLYGSQTPN EECLFLERLE ENHYVTYISK |
| | KHAEKNWFVG LKKNGRVKRG PRTHYGQKAI LFLPLPVSSD (SEQ ID NO: 406) |
| | Salk_053 FNLPPGNYKK PVLLYCSNGG HFLRILPDGT VDGTRDRSDQ |
| | HIQLQLSAES VGEVYIKSTE TGQYLAMDTD GLLYGSQTPN EECLFLERLE |
| | ENHYTTYISK KHAEKNWFVG LKKNGRVKRG PRTHYGQKAI LFLPLPVSSD (SEQ |
| | ID NO: 407) |
| | Salk_054 FNLPPGNYKK PKLLYCSNGG HFLRILPDGT VDGTRDRSDQ |
| | HIQLQLSAES VGEVAIKSTE TGQYLAMDTD GLLYGSQTPN EECLFLERLE |
| | ENHYNTYISK KHAEKNWFVG LKKNGRVKRG PRTHYGQKAI LFLPLPVSSD (SEQ |
| | ID NO: 408) |
| | Salk_055 FNLPPGNYKK PKLLYCSNGG HFLRILPDGT VDGTRDRSDQ |
| | HIQLQLSAES VGEVWIKSTE TGQYLAMDTD GLLYGSQTPN EECLFLERLE |
| | ENHYNTYISK KHAEKNWFVG LKKNGRVKRG PRTHYGQKAI LFLPLPVSSD (SEQ |
| | ID NO: 409) |
| | Salk_056 FNLPPGNYKK PKLLYCSNGG HFLRILPDGT VDGTRDRSDQ |
| | HIQLQLSAES VGEVYIKSTE TGQYLAMDTD GLLYGSQTPN EECLFLHRLE |
| | ENHYNTYISK KHAEKNWFVG LKKNGRVKRG PRTHYGQKAI LFLPLPVSSD (SEQ |
| | ID NO: 410) |
| | Salk_061 K PKLLYCSNGG HFLRILPDGT VDGTEDRSDQ HIQLQLSAES |
| | VGEVYIKSTE TGQYLAMDTD GLLYGSQTPN EECLFLERLE ENHYNTYISK |
| | KHAEKNWFVG LKKNGRVKRG PRTHYGQKAI LFLPLPVSSD (SEQ ID NO: 411) |
| | K PKLLYCSNGG HFLRILPDGT VDGTRDRSDQ HIQLQLSAES VGEVYIKSTE |
| | TGQYLAMDTDGLLYGSQTPN EECLFLERLE ENHYNTYISK KHAEKNWFVG |
| | LKKNGSVKRG PRTHYGQKAI LFLPLPVSSD (SEQ ID NO: 412) |
| | FNLPPGNYKK PKLLYCSNGG HFLRILPDGT VDGTRDRSDQ HIQLQLSAAS |
| | VGEVYIKSTE TGQYLAMDTD GLLYGSQTPN EECLFLERLE ENHYNTYISK |
| | KHAEKNWFVG LKKNGRVKRG PRTHYGQKAI LFLPLPVSSD (SEQ ID NO: 413) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHVVTYISK KHAEKNWFVG |
| | LKKNGRVKRG PRTHYGQKAI LFLPLPVSSD (SEQ ID NO: 414) |
| | K PVLLYCSNGG HFLRILPDGT VDGTRDRSDQ HIQLQLSAES VGEVYIKSTE |
| | TGQYLAMDTD GLLYGSQTPN EECLFLERLE ENHYVTYISK KHAEKNWFVG |
| | LKKNGRVKRG PRTHYGQKAI LFLPLPVSSD (SEQ ID NO: 415) |
| | FNLPPGNYKK PKLLYCSNGG HFLRILPDGT VDGTRDRSDQ HIQLQLSAES |
| | VGEVYIKSTE TGQYLAMDTDGLLYGSQTPN EECLFLERLE ENGYNTYISK |
| | KHAEKNWFVG LKKNGSCKRG PRTHYGQKAI LFLPLPVSSD (SEQ ID NO: 416) |
| | FNLPPGNYKK PKLLYCSNGG HFLRILPDGT VDGTRDRSDP HIQLQLIAES |
| | VGEVYIKSTE TGQYLAMDTDGLLYGSQTPN EECLFLERLE ENGYNTYISK |
| | KHAEKNWFVG LKKNGSCKRG PRTHYGQKAI LFLPLPVSSD (SEQ ID NO: 417) |
| | FNLPPGNYKK PVLLYCSNGG HFLRILPDGT VDGTRDRSDQ HIQLQLSAES |
| | VGEVYIKSTE TGQYLAMDTDGLLYGSQTPN EECLFLERLE ENHYNTYISK |
| | KHAEKNWFVG LKKNGSCKRG PRTHYGQKAI LFLPLPVSSD (SEQ ID NO: 418) |
| | FNLPPGNYKK PVLLYCSNGG HFLRILPDGT VDGTRDRSDQ HIQLQLSAES |
| | VGEVYIKSTE TGQYLAMDTDGLLYGSQTPN EECLFLERLE ENHYVTYISK |
| | KHAEKNWFVG LKKNGSCKRG PRTHYGQKAI LFLPLPVSSD (SEQ ID NO: 419) |
| | SYNHLQGDVR PVLLYCSNGG YFLRILPDGT VDGTRDRSDQ HIQFQLSAES |
| | VGEVYIKSTE TGQYLAMDTDGLLYGSQTPN EECLFLERLE ENHYVTYISK |
| | KYAEKNWYVG LKKNGSVKRG PRTHYGQKAI LFLPLPVSSD (SEQ ID NO: 420) |
| | SYNHLQGDVR VVLLYCSNGG YFLRILPDGT VDGTRDRSDQ HIQFQLSAES |
| | VGEVYIKSTE TGQYLAMDTD GLLYGSQTPN EECLFLERLE ENHYVTYISK |
| | KYAEKNWYVG LKKNGSVKRG PRTHYGQKAI LFLPLPVSSD (SEQ ID NO: 421) |
| | SYDYMEGGDIR VVLLYCSNGG YFLRILPDGT VDGTRDRSDQ HIQFQLSAES |
| | VGEVYIKSTE TGQYLAMDTD GLLYGSQTPN EECLFLERLE ENHYVTYISK |
| | KYAEKNWYVG LKKNGSVKRG PRTHYGQKAI LFLPLPVSSD (SEQ ID NO: 422) |

### Peptide Modifications

The mutant FGF1 protein or the FGF1-vagus targeting chimeric protein can be modified, e.g., to improve stability or its pharmacological profile. Exemplary chemical modifications include, e.g., adding chemical moieties, creating new bonds, and removing chemical moieties. Modifications at amino acid side groups include acylation of lysine ε-amino groups, N-alkylation of arginine, histidine, or lysine, alkylation of glutamic or aspartic carboxylic acid groups, and deamidation of glutamine or asparagine. Modifications of the terminal amino group include the des-amino, N-lower alkyl, N-di-lower alkyl, and N-acyl modifications. Modifications of the terminal carboxyl group include the amide, lower alkyl amide, dialkyl amide, and lower alkyl ester modifications.

In some embodiments, the mutant FGF1 protein or the FGF1-vagus targeting chimeric protein is linked to (e.g., attached to) a heparin molecule.

In some examples, the mutant FGF1 protein or the FGF1-vagus targeting chimeric protein is modified to include water soluble polymers, such as polyethylene glycol (PEG), PEG derivatives, polyalkylene glycol (PAG), polysialyic acid, or hydroxyethyl starch).

In some examples, the mutant FGF1 protein or the FGF1-vagus targeting chimeric protein is PEGylated at one or more positions, such as at N95 of FGF1 (for example see methods of Niu et al., J. Chromatog. 1327:66-72, 2014).

In some examples, the mutant FGF1 protein or the FGF1-vagus targeting chimeric protein includes an immunoglobin Fc domain (for example see Czajkowsky et al., EMBO Mol. Med. 4:1015-28, 2012, herein incorporated by reference). The conserved Fc fragment of an antibody can be incorporated either N-terminal or C-terminal of the protein, and can enhance stability of the protein and therefore serum half-life. The Fc domain can also be used as a means to purify the proteins on Protein A or Protein G sepharose beads.

### Variant Sequences

Proteins that vary in sequence from the disclosed mutant FGF1 proteins and variant FGF1-vagus targeting chimeric proteins, including variants of the sequences shown in Table 2, are provided herein. Such variants can contain one or more mutations, such as a single insertion, a single deletion, a single substitution. In one example, such variant peptides are produced by manipulating the nucleotide sequence encoding a peptide using standard procedures such as site-directed mutagenesis or PCR. Such variants can also be chemically synthesized.

In some examples, a mutant FGF1 protein includes 1-20 insertions, 1-20 deletions, 1-20 substitutions, and/or any combination thereof (e.g., single insertion together with 1-19 substitutions) as compared to a native FGF1 protein. In some examples, the disclosure provides a variant of any disclosed mutant FGF1 protein having 1, 2, 3, 4, 5, 6, 7, 8, 9, 10, 11, 12, 13, 14, 15, 16, 17, 18, 19 or 20 additional amino acid changes. In some examples, any mutant FGF1 protein provided in any of SEQ ID NOS: 10-422 (such as SEQ ID NO: 420, 421, or 422) includes 1-8 insertions, 1-15 deletions, 1-10 substitutions, and/or any combination thereof (e.g., 1-15, 1-4, or 1-5 amino acid deletions together with 1-10, 1-5 or 1-7 amino acid substitutions). In some examples, the disclosure provides a variant of any one of SEQ ID NOS: 10-422, having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes.

In some examples, an FGF1-vagus targeting chimeric protein includes 1-20 insertions, 1-20 deletions, 1-20 substitutions, and/or any combination thereof (e.g., single insertion together with 1-19 substitutions) as compared to a FGF1-vagus targeting chimeric protein provided herein. In some examples, the disclosure provides a variant of any disclosed FGF1-vagus targeting chimeric protein having 1, 2, 3, 4, 5, 6, 7, 8, 9, 10, 11, 12, 13, 14, 15, 16, 17, 18, 19 or 20 additional amino acid changes. In some examples, any FGF1-vagus targeting chimeric protein provided in any of SEQ ID NOS: 424, 425, 426, 427, 428, 429, 430, 431, 432, or 433 includes 1-8 insertions, 1-15 deletions, 1-10 substitutions, and/or any combination thereof (e.g., 1-15, 1-4, or 1-5 amino acid deletions together with 1-10, 1-5 or 1-7 amino acid substitutions). In some examples, the disclosure provides a variant of any one of SEQ ID NOS: 424, 425, 426, 427, 428, 429, 430, 431, 432, or 433, having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes.

One type of modification or mutation that can be made is the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in any one of SEQ ID NOS: 10-422 or 424, 425, 426, 427, 428, 429, 430, 431, 432, or 433, which does not substantially affect the ability of the peptide to decrease blood glucose in a mammal. An alanine scan can be used to identify which amino acid residues in a mutant FGF1 protein (such as any one of SEQ ID NOS: 10-422) or an FGF1-vagus targeting chimeric proteins (such as any one of SEQ ID NOS: 424, 425, 426, 427, 428, 429, 430, 431, 432, or 433) can tolerate an amino acid substitution. In one example, the blood glucose lowering activity of any one of SEQ ID NOS: 10-422, 424, 425, 426, 427, 428, 429, 430, 431, 432, or 433 is not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys, Gln, or Asn for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or He for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions and/or additions) can be assessed by analyzing the function of the variant protein, such as any one of SEQ ID NOS: 10-422, 424, 425, 426, 427, 428, 429, 430, 431, 432, or 433, by analyzing the ability of the variant protein to decrease blood glucose in a mammal

### Generation of Proteins

Isolation and purification of recombinantly expressed mutated FGF1 proteins and FGF1-vagus targeting chimeric proteins can be carried out by conventional means, such as preparative chromatography and immunological separations. Once expressed, mutated FGF1 proteins or FGF1-vagus targeting chimeric proteins can be purified according to standard procedures, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, mutated FGF1 proteins and FGF1-vagus targeting chimeric proteins disclosed herein can also be constructed in whole or in part using standard peptide synthesis. In one example, mutated FGF1 proteins or FGF1-vagus targeting chimeric proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### Nucleic Acid Molecules and Vectors

Nucleic acid molecules encoding a mutated FGF1 protein or an FGF1-vagus targeting chimeric protein are encompassed by this disclosure. Based on the genetic code, nucleic acid sequences coding for any mutated FGF1 sequence or any FGF1-vagus targeting chimeric protein, can be routinely generated. In some examples, such a sequence is optimized for expression in a host cell, such as a host cell used to express the mutant FGF1 protein or the FGF1-vagus targeting chimeric protein.

A nucleic acid sequence that codes for a mutant FGF1 protein having at least 60%, at least 70%, at least 75%, at least 80%, at least 90%, at least 92%, at least 95%, at least 96%, at least 97%, at least 98%, at least 99%, or 100% sequence identity to any one of SEQ ID NOS: 10-422 (such as to SEQ ID NO: 420, 421, or 422), can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same mutant

FGF1 protein sequence. Similarly, a nucleic acid sequence that codes for an FGF1-vagus targeting chimeric protein having at least 60%, at least 70%, at least 75%, at least 80%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433, can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same FGF1-vagus targeting chimeric protein.

Nucleic acid molecules include DNA, cDNA, and RNA sequences which encode a mutated FGF1 peptide or a FGF1-vagus targeting chimeric protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a mutated FGF1 protein (such as one encoding a protein generated using the mutations shown in Table 1, the sequences in any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422) or encoding FGF1-vagus targeting chimeric protein (such as the sequences in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) that take advantage of the codon usage preferences of that particular species. For example, the proteins disclosed herein can be designed to have codons that are preferentially used by a particular organism of interest.

A nucleic acid encoding the desired protein can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). A wide variety of cloning and in vitro amplification methodologies are well known. In addition, nucleic acids encoding sequences encoding a desired protein can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding a desired protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In one example, a mutant FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOs: 10-422, such as to SEQ ID NO: 420, 421, or 422) or FGF1-vagus targeting chimeric protein (such as a sequence in any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is prepared by inserting the cDNA which encodes the protein into a vector. The insertion can be made so that the desired protein is read in frame and produced.

The mutated FGF1 nucleic acid coding sequence (such as a sequence encoding any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422) or a FGF1-vagus targeting chimeric protein coding sequence (such as a sequence encoding any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant, and mammalian organisms. Methods of expressing DNA sequences having eukaryotic or viral sequences in prokaryotes are well known in the art. Biologically functional viral and plasmid DNA vectors capable of expression and replication in a host are known in the art. The vector can encode a selectable marker, such as a thymidine kinase gene.

Nucleic acid sequences encoding a mutated FGF1 protein (such as encoding a sequence in any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOs: 10-422) or encoding a FGF1-vagus targeting chimeric protein (such as encoding a sequence in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be operatively linked to expression control sequences. An expression control sequence operatively linked to a desired protein coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a mutated FGF1 protein- or FGF-vagus targeting chimera-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHOS (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a mutated FGF1 protein (such as a sequence encoding any one of SEQ ID NOs: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422) or encoding a FGF1-vagus targeting chimeric protein (such as encoding a sequence in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can also be designed to express in insect cells.

A mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422) or a FGF1-vagus targeting chimeric protein (such as the sequences in any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be expressed in a variety of yeast strains. For example, seven pleiotropic drug-resistant transporters, YOR1, SNQ2, PDR5, YCF1, PDR10, PDR]], and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a mutated FGF1 protein (such as a sequence in any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) or that encode a FGF1-vagus targeting chimeric protein (such as the sequences in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433). Exemplary viral vectors include polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, and poliovirus vectors. Specific exemplary vectors are poxvirus vectors such as vaccinia virus, fowlpox virus and a highly attenuated vaccinia virus (MVA), adenovirus, baculovirus, and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox, and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a mutated FGF1 protein (such as any one of SEQ ID NOS: 10-422, or encode a protein having at least 80%, at least 85%, at least 90%, at least 92%, at least 95%, at least 96%, at least 97%, at least 98%, at least 99%, or 100% sequence identity to any one of SEQ ID NOS: 10-422) or that encode a FGF1-vagus targeting chimeric protein (such as the sequences in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can include at least one expression control element operationally linked to the nucleic acid sequence encoding the protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the protein in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding a mutated FGF1 protein (such as any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422) or encoding a FGF1-vagus targeting chimeric protein (such as the sequences in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) are known. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitation, conventional mechanical procedures such as microinjection, electroporation, insertion of a plasmid encased in liposomes, or virus vectors can be used. Eukaryotic cells can also be co-transformed with polynucleotide sequences encoding a mutated FGF1 protein (such as any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422) or encoding a FGF1-vagus targeting chimeric protein (such as the sequences in any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression system such as plasmids and vectors of use in producing proteins in cells including higher eukaryotic cells such as the COS, CHO, HeLa and myeloma cell lines.

### Recombinant Cells

A nucleic acid molecule encoding a mutated FGF1 protein or an FGF1-vagus targeting chimeric protein disclosed herein can be used to transform cells and make transformed cells. Thus, cells expressing a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) or an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), are disclosed. Cells expressing a mutated FGF1 protein or an FGF1-vagus targeting chimeric protein disclosed herein can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines.

Cells expressing a mutated FGF1 protein or an FGF1-vagus targeting chimeric protein are transformed or recombinant cells. Such cells can include at least one exogenous nucleic acid molecule that encodes a mutated FGF1 protein, for example any of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422, or that encodes a FGF1-vagus targeting chimeric protein (such as any one of SEQ ID NOS: 424, 425, 426, 427, 428, 429, 430, 431, 432, or 433, or those encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433). It is understood that all progeny may not be identical to the parental cell since there may be mutations that occur during replication. Methods of stable transfer, meaning that the foreign DNA is continuously maintained in the host cell, are known in the art.

Transformation of a host cell with recombinant DNA may be carried out by conventional techniques as are well known. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. Techniques for the propagation of mammalian cells in culture are well-known (see, Jakoby and Pastan (eds.), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation, and gene guns are also known in the art.

### Pharmaceutical Compositions

Pharmaceutical compositions that include a mutated FGF1 protein (such as any one of SEQ ID NOS: 10-422, or a protein having at least at least 80%, at least 85%, 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), an FGF1-vagus targeting chimeric protein (such any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid encoding such as protein, can be formulated with an appropriate pharmaceutically acceptable carrier, depending upon the particular mode of administration chosen.

In some embodiments, the pharmaceutical composition consists essentially of at least one mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) (or a nucleic acid encoding such a protein), at least one FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) (or a nucleic acid encoding such a protein), or combinations thereof, and a pharmaceutically acceptable carrier. In these embodiments, additional therapeutically effective agents are not included in the compositions.

In one embodiment, the pharmaceutical composition includes at least one mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) (or a nucleic acid encoding such a protein), at least one FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) (or a nucleic acid encoding such a protein), or combinations thereof, and a pharmaceutically acceptable carrier. Additional therapeutic agents, such as agents for the treatment of diabetes, can be included. Thus, the pharmaceutical compositions can include a therapeutically effective amount of another agent. Examples of such agents include, without limitation, anti-apoptotic substances such as the Nemo-Binding Domain and compounds that induce proliferation such as cyclin dependent kinase (CDK)-6, CDK-4 and cyclin Dl. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570, aleglitazar, farglitazar, muraglitazar, tesaglitazar, and TZD) and PPAR-γ antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. Additional examples include immunomodulatory factors such as anti-CD3 mAb, growth factors such as HGF, VEGF, PDGF, lactogens, and PTHrP. In some examples, the pharmaceutical compositions containing a mutated FGF1 protein and/or an FGF1-vagus targeting chimeric protein can further include a therapeutically effective amount of other FGFs, such as FGF21, FGF19, or both, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) and/or an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) or an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

With regard to the administration of nucleic acids, one approach to administration of nucleic acids is direct treatment with plasmid DNA, such as with a mammalian expression plasmid. As described above, the nucleotide sequence encoding a mutated FGF1 protein (such as encoding any one of SEQ ID NOS: 10-422, or encoding a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or encoding an FGF1-vagus targeting chimeric protein (such as encoding any one of SEQ ID NOS: 424, 425, 426, 427, 428, 429, 430, 431, 432, or 433, or encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), can be placed under the control of a promoter to increase expression of the protein.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which a mutated FGF1 protein (such as a protein in any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or polynucleotide encoding such a protein, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant can be suitable for treatment of chronic conditions, such as diabetes. Long-term release, as used herein, means that the implant is constructed and arranged to deliver therapeutic levels of the active ingredient for at least 30 days, and preferably 60 days. Long-term sustained release implants are well known and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of the disclosed mutated FGF1 proteins and FGF1-vagus targeting chimeric proteins, such as the inclusion of a C-terminal amide, can be used.

The dosage form of the pharmaceutical composition can be determined by the mode of administration chosen. For instance, in addition to injectable fluids, topical, inhalation, oral, and suppository formulations can be employed. Topical preparations can include eye drops, ointments, sprays, patches, and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), and/or an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be formulated in unit dosage form, suitable for individual administration of precise dosages.

In one non-limiting example, a unit dosage contains from about 1 mg to about 1 g of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) is about 0.01 mg/kg to about 50 mg/kg, for example, about 0.5 mg/kg to about 25 mg/kg, about 0.5 mg/kg to about 1 mg/kg, about 0.5 mg/kg to about 5 mg/kg, about 0.05 mg/kg to about 0.1 mg/kg, about 0.01 mg/kg to about 0.1 mg/kg, or about 1 mg/kg to about 10 mg/kg. In other examples, a therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) is about 1 mg/kg to about 5 mg/kg, for example about 2 mg/kg. In a particular example, a therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) includes about 1 mg/kg to about 10 mg/kg, such as about 2 mg/kg. In a particular example, a therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) includes about 0.01 mg/kg to about 0.5 mg/kg, such as about 0.1 mg/kg, 0.5 mg/kg, 0.63 mg/kg, or 1 mg/kg.

In one non-limiting example, a unit dosage contains from about 1 mg to about 1 g of an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is about 0.01 mg/kg to about 50 mg/kg, for example, about 0.5 mg/kg to about 25 mg/kg, about 0.5 mg/kg to about 1 mg/kg, about 0.5 mg/kg to about 5 mg/kg, about 0.05 mg/kg to about 0.1 mg/kg, about 0.01 mg/kg to about 0.1 mg/kg, or about 1 mg/kg to about 10 mg/kg. In other examples, a therapeutically effective amount of an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is about 1 mg/kg to about 5 mg/kg, for example about 2 mg/kg. In a particular example, a therapeutically effective amount of an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) includes about 1 mg/kg to about 10 mg/kg, such as about 2 mg/kg. In a particular example, a therapeutically effective amount of an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) includes about 0.01 mg/kg to about 0.5 mg/kg, such as about 0.1 mg/kg, as about 0.1 mg/kg, 0.5 mg/kg, 0.63 mg/kg, or 1 mg/kg.

### Methods of Treatment

The disclosed mutated FGF1 proteins (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric proteins (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or nucleic acids encoding such proteins, can be administered to a subject, for example to treat a metabolic disease, for example by reducing fed and fasting blood glucose, improving insulin sensitivity and glucose tolerance, reducing systemic chronic inflammation, ameliorating hepatic steatosis in a mammal, reducing food intake, or combinations thereof.

The compositions of this disclosure that include a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433, or nucleic acids encoding these proteins, can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), an FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid encoding such a protein, to pancreas tissue (for example by using a pump, or by implantation of a slow release form at the site of the pancreas). The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly, every other week, monthly, every 7 days, every 10 days, every 14 days, every 21 days, every 30 days, every 40 days, every 60 days, etc.)

doses of the mutant FGF1 or FGF1-vagus targeting chimera over a period of a few days, few weeks, to months, or even years. It is shown herein that the FGF1-vagus targeting chimeras can achieve long-lasting glucose lowering effects. For example, a therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422) and/or FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be administered in a single dose, twice daily, weekly, every other week, every three weeks, every month, every other month, or in several doses, for example daily, or during a course of treatment. In a particular non-limiting example, treatment involves once daily dose, twice daily dose, once weekly dose, every other week dose, or monthly dose.

The amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or FGF1-vagus targeting chimeric proteins (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), administered can be dependent on the subject being treated, the severity of the affliction, and the manner of administration, and is best left to the judgment of the prescribing clinician. Determination of the appropriate amount to be administered is within the routine level of ordinary skill in the art. Within these bounds, the formulation to be administered will contain a quantity of the mutated FGF1 protein and/or the FGF1-vagus targeting chimeric protein in amounts effective to achieve the desired effect in the subject being treated. A therapeutically effective amount of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or FGF1-vagus targeting chimeric proteins (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be the amount of the protein (or a nucleic acid encoding these proteins) that is necessary to treat diabetes or reduce blood glucose levels (for example a reduction of at least 5%, at least 10% or at least 20%, for example relative to no administration of the mutant FGF1 or the FGF1-vagus targeting chimera).

When a viral vector is utilized for administration of a nucleic acid encoding a mutated FGF1 protein (such as any one of SEQ ID NOS: 10-422, or those encoding a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or encoding a FGF1-vagus targeting chimeric protein (such as any one of SEQ ID NOS: 424, 425, 426, 427, 428, 429, 430, 431, 432, or 433, or encoding a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), the recipient can receive a dosage of each recombinant virus in the composition in the range of from about 10⁵ to about 10¹⁰ plaque forming units/mg mammal, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal, or intramuscular administration of the virus. Alternatively the recombinant viral vector or combination of recombinant viral vectors may be administered locally by direct injection into the pancreas in a pharmaceutically acceptable carrier.

Generally, the quantity of recombinant viral vector, carrying the nucleic acid sequence of the mutated FGF1 protein to be administered (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or the FGF1-vagus targeting chimeric protein to be administered (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) is based on the titer of virus particles. An exemplary range to be administered is 10⁵ to 10¹⁰ virus particles per mammal, such as a human

In some examples, a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric proteins (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid encoding the mutated FGF1 protein or the FGF1-vagus targeting chimera, is administered in combination (such as sequentially or simultaneously or contemporaneously) with one or more other agents, such as those useful in the treatment of diabetes or insulin resistance (e.g., insulin).

Anti-diabetic agents are generally categorized into six classes: biguanides (e.g., metformin); thiazolidinediones (including rosiglitazone (Avandia®), pioglitazone (Actos®), rivoglitazone, and troglitazone); sulfonylureas; inhibitors of carbohydrate absorption; fatty acid oxidase inhibitors and anti-lipolytic drugs; and weight-loss agents. Any of these agents can also be used in the methods disclosed herein. The anti-diabetic agents include those agents disclosed in Diabetes Care, 22(4):623-634. One class of anti-diabetic agents of use is the sulfonylureas, which are believed to increase secretion of insulin, decrease hepatic glucogenesis, and increase insulin receptor sensitivity. Another class of anti-diabetic agents is the biguanide antihyperglycemics, which decrease hepatic glucose production and intestinal absorption, and increase peripheral glucose uptake and utilization, without inducing hyperinsulinemia.

In some examples, a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), or a FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433) can be administered in combination with effective doses of anti-diabetic agents (such as biguanides, thiazolidinediones, or incretins) and/or lipid lowering compounds (such as statins or fibrates). The terms “administration in combination,” “co-administration,” or the like, refer to both concurrent and sequential administration of the active agents. Administration of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), a FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid encoding such a protein, may also be in combination with lifestyle modifications, such as increased physical activity, low fat diet, low sugar diet, and smoking cessation.

Additional agents that can be used in combination with the disclosed mutated FGF1 proteins and FGF1-vagus targeting chimeric proteins include, without limitation, anti-apoptotic substances such as the Nemo-Binding Domain and compounds that induce proliferation such as cyclin dependent kinase (CDK)-6, CDK-4 and Cyclin D1. Other active agents can be utilized, such as antidiabetic agents for example, insulin, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570) and antagonists, PPAR-gamma/alpha modulators (such as KRP 297), alpha-glucosidase inhibitors (e.g., acarbose, voglibose), Dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g., exendin-4) or amylin. In some embodiments the agent is an immunomodulatory factor such as anti-CD3 mAb, growth factors such as HGF, vascular endothelial growth factor (VEGF), platelet derived growth factor (PDGF), lactogens, or parathyroid hormone related protein (PTHrP). In one example, the mutated FGF1 protein and/or the FGF1-vagus targeting chimeric protein is administered in combination with a therapeutically effective amount of another FGF, such as FGF21, FGF19, or both, heparin, or combinations thereof.

In some embodiments, methods are provided for treating diabetes or pre-diabetes in a subject by administering a therapeutically effective amount of a composition including a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid encoding the \ protein, to the subject. The subject can have diabetes type I or diabetes type II. The subject can be any mammalian subject, including human subjects and veterinary subjects such as cats and dogs. The subject can be a child or an adult. The subject can also be administered insulin. The method can include measuring blood glucose levels.

In some examples, the method includes selecting a subject with diabetes, such as type I or type II diabetes, or a subject at risk for diabetes, such as a subject with pre-diabetes. These subjects can be selected for treatment with the disclosed mutated FGF1 proteins (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or nucleic acid molecules encoding such.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010).

In some examples, the subject treated with the disclosed compositions and methods has HbA1C of greater than 6.5% or greater than 7%.

In some examples, treating diabetes includes one or more of increasing glucose tolerance (such as an increase of at least 5%, at least 10%, at least 20%, or at least 50%, for example relative to no administration of the mutant FGF1 or the FGF1-vagus targeting chimera), decreasing insulin resistance (for example, decreasing plasma glucose levels, decreasing plasma insulin levels, or a combination thereof, such as decreases of at least 5%, at least 10%, at least 20%, or at least 50%, for example relative to no administration of the mutant FGF1 or the FGF1-vagus targeting chimera), decreasing serum triglycerides (such as a decrease of at least 10%, at least 20%, or at least 50%, for example relative to no administration of the mutant FGF1 or the FGF1-vagus targeting chimera), decreasing free fatty acid levels (such as a decrease of at least 5%, at least 10%, at least 20%, or at least 50%, for example relative to no administration of the mutant FGF1 or the FGF1-vagus targeting chimera), and decreasing HbA1c levels in the subject (such as a decrease of at least 0.5%, at least 1%, at least 1.5%, at least 2%, or at least 5% for example relative to no administration of the mutant FGF1 or the FGF1-vagus targeting chimera). In some embodiments, the disclosed methods include measuring glucose tolerance, insulin resistance, plasma glucose levels, plasma insulin levels, serum triglycerides, free fatty acids, and/or HbA1c levels in a subject.

In some examples, administration of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or nucleic acid molecule encoding such, treats a metabolic disease, such as diabetes (such as type II diabetes) or pre-diabetes, by decreasing of HbA1C, such as a reduction of at least 0.5%, at least 1%, or at least 1.5%, such as a decrease of 0.5% to 0.8%, 0.5% to 1%, 1 to 1.5% or 0.5% to 2%. In some examples the target for HbA1C is less than about 6.5%, such as about 4-6%, 4-6.4%, or 4-6.2%. In some examples, such target levels are achieved within about 26 weeks, within about 40 weeks, or within about 52 weeks. Methods of measuring HbA1C are routine, and the disclosure is not limited to particular methods. Exemplary methods include HPLC, immunoassays, and boronate affinity chromatography.

In some examples, administration of a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or nucleic acid molecule encoding such, treats diabetes or pre-diabetes by increasing glucose tolerance, for example, by decreasing blood glucose levels (such as two-hour plasma glucose in an OGTT or FPG) in a subject. In some examples, the method includes decreasing blood glucose by at least 5% (such as at least 10%, at least 15%, at least 20%, at least 25%, at least 30%, at least 35%, or more) as compared with a control (such as no administration of any of insulin, a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID

NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid molecule encoding such). In particular examples, a decrease in blood glucose level is determined relative to the starting blood glucose level of the subject (for example, prior to treatment with a mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or nucleic acid molecule encoding such). In other examples, decreasing blood glucose levels of a subject includes reduction of blood glucose from a starting point (for example greater than about 126 mg/dL FPG or greater than about 200 mg/dL OGTT two-hour plasma glucose) to a target level (for example, FPG of less than 126 mg/dL or OGTT two-hour plasma glucose of less than 200 mg/dL). In some examples, a target FPG may be less than 100 mg/dL. In other examples, a target OGTT two-hour plasma glucose may be less than 140 mg/dL. Methods to measure blood glucose levels in a subject (for example, in a blood sample from a subject) are routine.

In other embodiments, the disclosed methods include comparing one or more indicators of diabetes (such as glucose tolerance, triglyceride levels, free fatty acid levels, or HbA1c levels) to a control (such as no administration of any of insulin, any mutated FGF1 protein (such as any one of SEQ ID NOS: 10-422, or a protein having at least 80%, at least 85%, at least 90%, at least 92%, at least 95%, at least 96%, at least 97%, at least 98%, at least 99%, or 100% sequence identity to any one of SEQ ID NOS: 10-422, such as to SEQ ID NO: 420, 421, or 422), any FGF1-vagus targeting chimeric protein (such as any one of SEQ ID NOS: 424, 425, 426, 427, 428, 429, 430, 431, 432, or 433, or a protein having at least 80%, at least 85%, at least 90%, at least 92%, at least 95%, at least 96%, at least 97%, at least 98%, at least 99%, or 100% sequence identity to any one of SEQ ID NOS: 424, 425, 426, 427, 428, 429, 430, 431, 432, or 433), or a nucleic acid molecule encoding such), wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

The disclosure is illustrated by the following non-limiting Examples.

### EXAMPLE 1

### Preparation of Proteins

Mutated FGF1 proteins, as well as FGF1-vagus targeting chimeric proteins, can be made using known methods (e.g., see Xia et al., PLoS One. 7(11):e48210, 2012). An example is provided below.

Briefly, a nucleic acid sequence encoding an FGF1 native or mutant protein (e.g., any of SEQ ID NOS: 5-422), or a nucleic acid sequence encoding an FGF1-vagus targeting chimeric protein (e.g., any of SEQ ID NOS: 424-433), can be fused downstream of an enterokinase (EK) recognition sequence (Asp₄Lys) preceded by a flexible 20 amino acid linker (derived from the S-tag sequence of pBAC-3) and an N-terminal (His)₆ tag. The resulting expressed fusion protein utilizes the (His)₆ tag for efficient purification and can be subsequently processed by EK digestion to yield the protein.

The protein can be expressed from an E. coli host after induction with isopropyl-β-D-thio-galactoside. The expressed protein can be purified utilizing sequential column chromatography on Ni-nitrilotriacetic acid (NTA) affinity resin followed by ToyoPearl HW-40S size exclusion chromatography. The purified protein can be digested with EK to remove the N-terminal (His)₆ tag, 20 amino acid linker, and (Asp4Lys) EK recognition sequence. A subsequent second Ni-NTA chromatographic step can be utilized to remove the released N-terminal protein (along with any uncleaved fusion protein). Final purification can be performed using HiLoad Superdex 75 size exclusion chromatography equilibrated to 50 mM Na₂PO₄, 100 mM NaCl, 10 mM (NH₄)₂SO₄, 0.1 mM ethylenediaminetetraacetic acid (EDTA), 5 mM L-Methionine, pH at 6.5 (“PBX” buffer); L-Methionine can be included in PBX buffer to limit oxidization of reactive thiols and other potential oxidative degradation.

In some examples, the enterokinase is not used, and instead, a protein (such as one that includes an N-terminal methionine) can be made and purified using heparin affinity chromatography.

For storage and use, the purified protein can be sterile filtered through a 0.22 micron filter, purged with N₂, snap frozen in dry ice and stored at −80° C. prior to use. The purity of the resulting protein can be assessed by both Coomassie Brilliant Blue and Silver Stain Plus (BIO-RAD Laboratories, Inc., Hercules Calif.) stained sodium dodecyl sulfate polyacrylamide gel electrophoresis (SDS PAGE). Proteins can be prepared in the absence of heparin. Prior to IV bolus, heparin, or PBS, can be added to the therapeutic protein.

In some examples, an FGF1 protein (e.g., any one of SEQ ID NOS: 5-422), or an FGF1-vagus targeting chimeric protein (e.g., any of SEQ ID NOS: 424-433), can be expressed in Escherichia coli cells and purified from the soluble bacterial cell lysate fraction by heparin affinity, ion exchange, and size exclusion chromatography.

### EXAMPLE 2

### Testing Proteins for Glucose Lowering Ability and Mitogenic Activity

This example describes methods for measuring the ability of the FGF1 mutant and native proteins provided herein (e.g., any of SEQ ID NOS: 5-422, or variants thereof), and FGF1-vagus targeting chimeric proteins (e.g., any of SEQ ID NOS: 424-433, or variants thereof), to lower blood glucose or treat a metabolic disease in vivo. In vitro mitogenic assays are also described. Similar methods can be used to test other FGF1 mutant proteins and other FGF1-vagus targeting chimeric proteins. Other exemplary methods are provided in Scarlett et al., Nat. Med. 22:800, 2016).

### Animals

Mice are housed in a temperature-controlled environment with a 12-hour light/12-hour dark cycle and handled according to institutional guidelines complying with U.S. legislation. Male ob/ob mice (B6.V-Lep^{ob}/J, Jackson laboratories) and male C57BL/6J mice receive a standard or high fat diet (MI laboratory rodent diet 5001, Harlan Teklad; high fat (60%) diet F3282, Bio-Serv) and acidified water ad libitum. Mice are injected subcutaneously with 0.1 to 1 mg/ml (such as 0.1, 0.25, 0.5, 0.63, or 1 mg/ml) solutions in PBS of the FGF1 protein (e.g., any of SEQ ID NOS: 5-422) or the FGF1-vagus targeting chimeric protein (e.g., any of SEQ ID NOS: 424-433) or PBS alone.

### Serum Analysis

Blood is collected by tail bleeding either in the ad libitum fed state or following overnight fasting.

Glucose tolerance tests (GTT) were conducted on overnight (10 hour) fasted ob/ob mice. Glucose (1 g/kg i.p.) was injected intraperitoneally and blood glucose monitored from tail bleeds using a OneTouch glucometer at the indicated times.

Pyruvate tolerance test (PTT) was performed on overnight fasted (16 hours) ob/ob mice. 1.5 mg g⁻¹ body weight sodium pyruvate in PBS was given intraperitoneally, and blood glucose monitored from tail bleeds using a OneTouch glucometer at the indicated times

Serum insulin levels were determined by a commercial enzyme linked immunosorbent assay (ELISA) according to manufacturer's manual (Millipore).

### Results

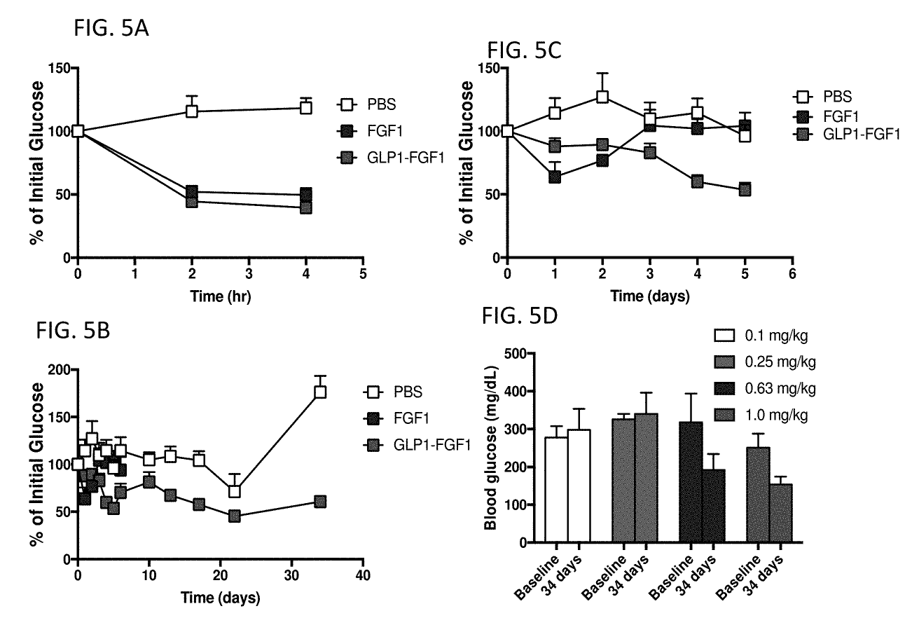

A native FGF1 protein (SEQ ID NO: 5), and an FGF1-vagus targeting chimeric protein (SEQ ID NO: 424) were generated and administered to diabetic ob/ob mice parenterally (subcutaneously), and the blood glucose lowering ability monitored over 34 days. FIGS. 5A-5C show the blood glucose lowering ability of mature FGF1 (SEQ ID NO: 5, as compared to an FGF1-vagus targeting chimeric protein (SEQ ID NO: 424), with values normalized to time zero. Equal molar amounts of the FGF1 protein was administered (0.5 mg/kg of FGF1 and 0.63 mg/kg of the FGF1-vagus targeting chimeric protein). At 4 hours, both FGF1 and the FGF1-vagus targeting chimeric protein showed similar amounts of blood glucose lowering activity. But as shown in FIGS. 5B and 5C, when monitored for longer periods of time, the FGF1-vagus targeting chimeric protein lowered blood glucose for a longer period of time. For example, as shown in FIG. 5B, by 5 days, glucose levels returned to pre-injection levels with FGF1, but remained lower with the chimeric protein. Thus, the FGF1-vagus targeting chimeric protein induced sustained glucose lowering. FIG. 5D shows the dose response results at 34 days after administration.

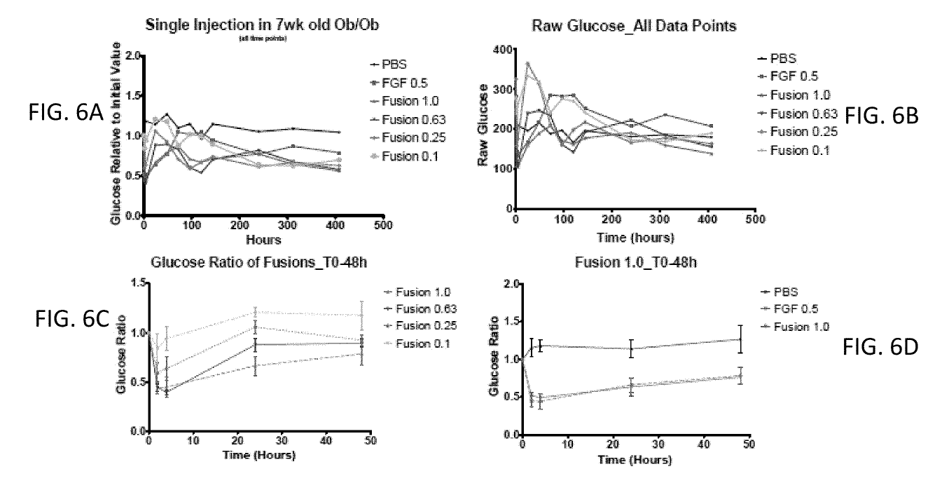

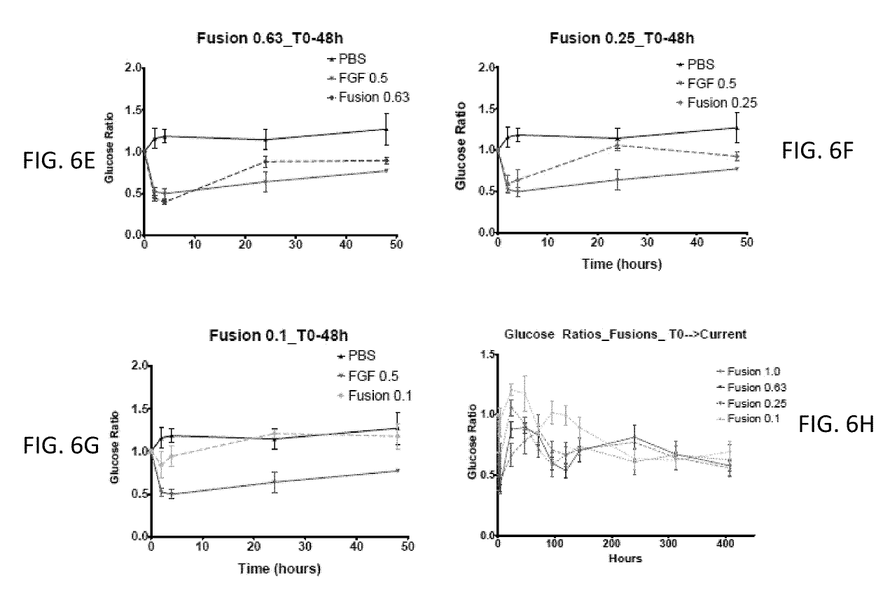

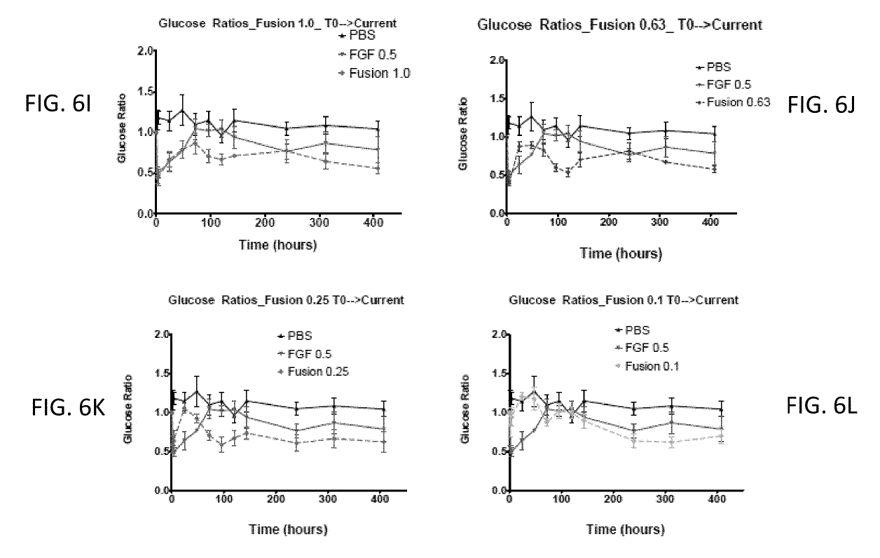

FIG. 6A shows the relative change in blood glucose (as compared to the initial blood glucose reading) over 400 hours following a single injection of PBS, FGF1, or FGF1-vagus targeting chimeric protein (fusion) (SEQ ID NO: 424, FIB. 3B). FIG. 6B shows the raw blood glucose readings over 400 hours following a single injection of PBS, FGF1, or FGF1-vagus targeting chimeric protein (fusion) (at 0.1, 0.25, 0.63, or 1 mg/kg).

FIGS. 6C to 6F show the glucose levels normalized to the glucose level prior to injection (glucose ratio) for 48 hours following administration of the PBS, FGF1, or FGF1-vagus targeting chimeric protein. FIGS. 6G to 6L show the glucose levels normalized to the glucose level prior to injection (glucose ratio) for 400 hours following administration of the PBS, FGF1, or FGF1-vagus targeting chimeric protein

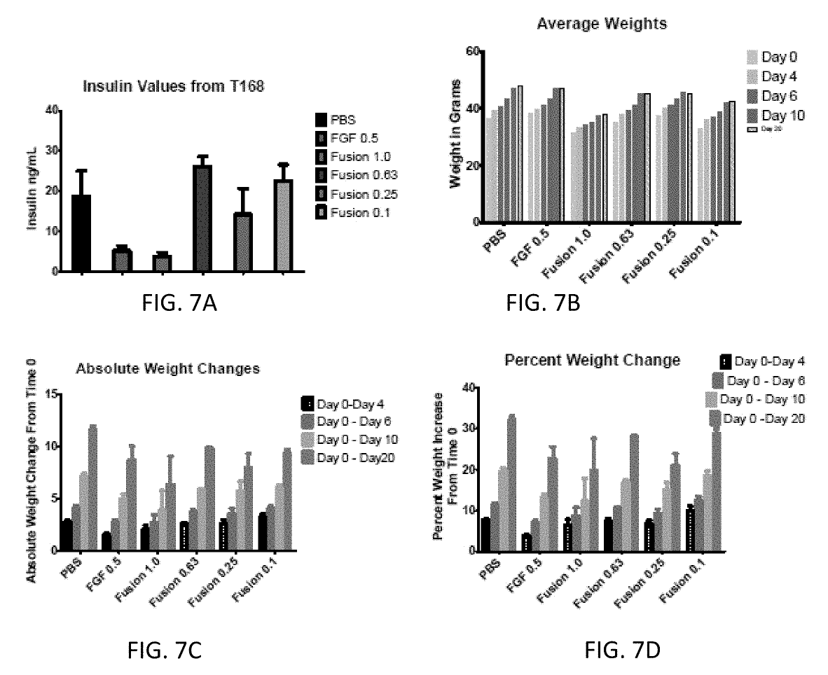

FIG. 7A shows the effect on insulin levels 168 hours following a single injection of PBS, FGF1, or FGF1-vagus targeting chimeric protein (fusion) (at 0.1, 0.25, 0.63, or 1 mg/kg). Administration of FGF1 at 0.5 mg/ml or FGF1-vagus targeting chimeric protein (fusion) at 1 mg/kg significantly reduced insulin levels.

FIGS. 7B-7D show the effect on weight 0 to 10 days following a single injection of PBS, FGF1, or FGF1-vagus targeting chimeric protein (fusion) (at 0.1, 0.25, 0.63, or 1 mg/kg). The weight of the treated mice did not change significantly during the treatment period, indicating that the glucose lowering effects were not significantly due to weight loss.

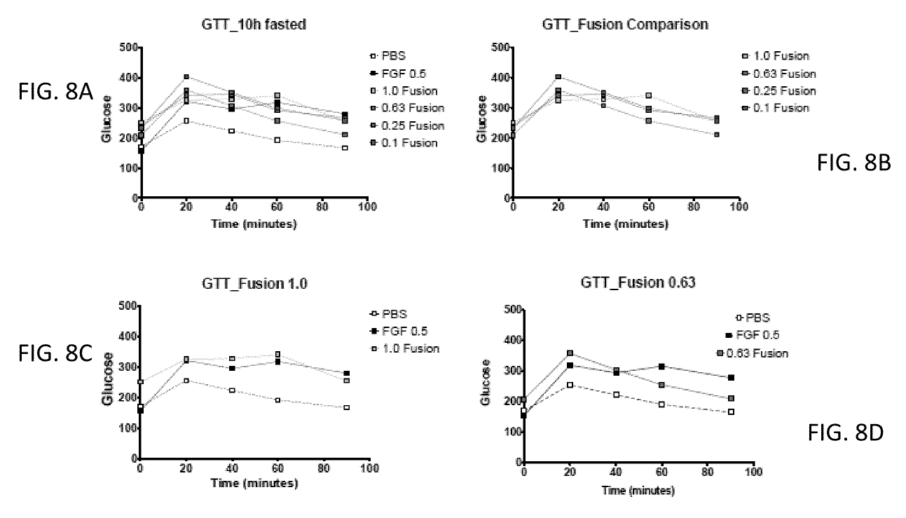

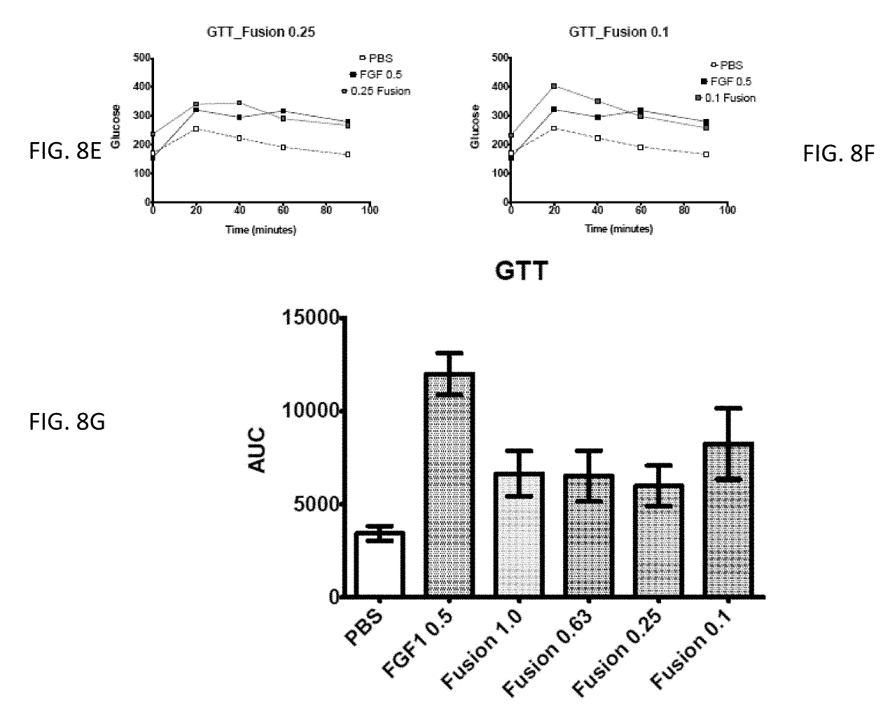

FIGS. 8A-8G show the change in fasting blood glucose over 90 minutes at day 15 following a single injection of PBS, FGF1, or FGF1-vagus targeting chimeric protein (fusion) and 10 hours of fasting. Single injections of FGF1 or FGF1-vagus targeting chimeric proteins (fusion) are not sufficient to significantly improve insulin sensitivity, as measured by glucose tolerance tests (GTTs) by day 15. Instead, treatment is needed for 3 weeks before improvement in GTT is observed.

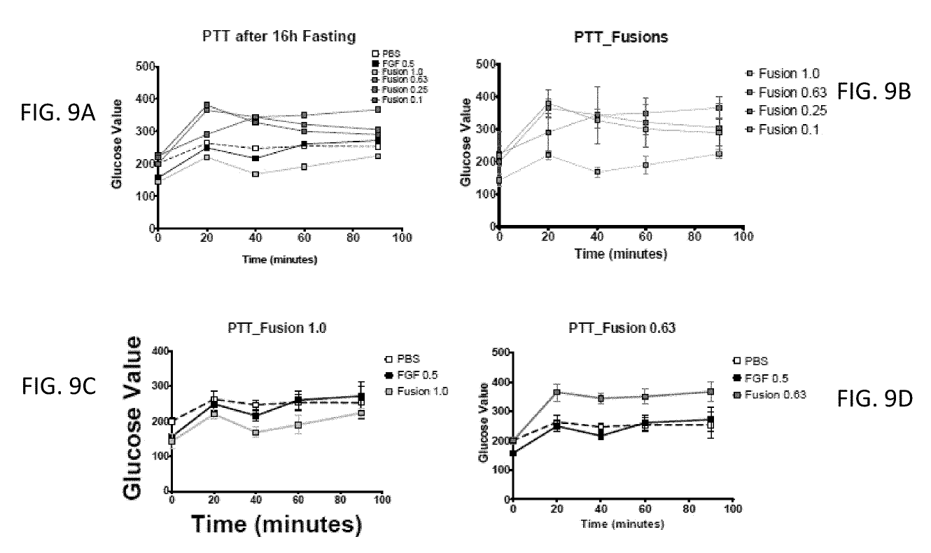

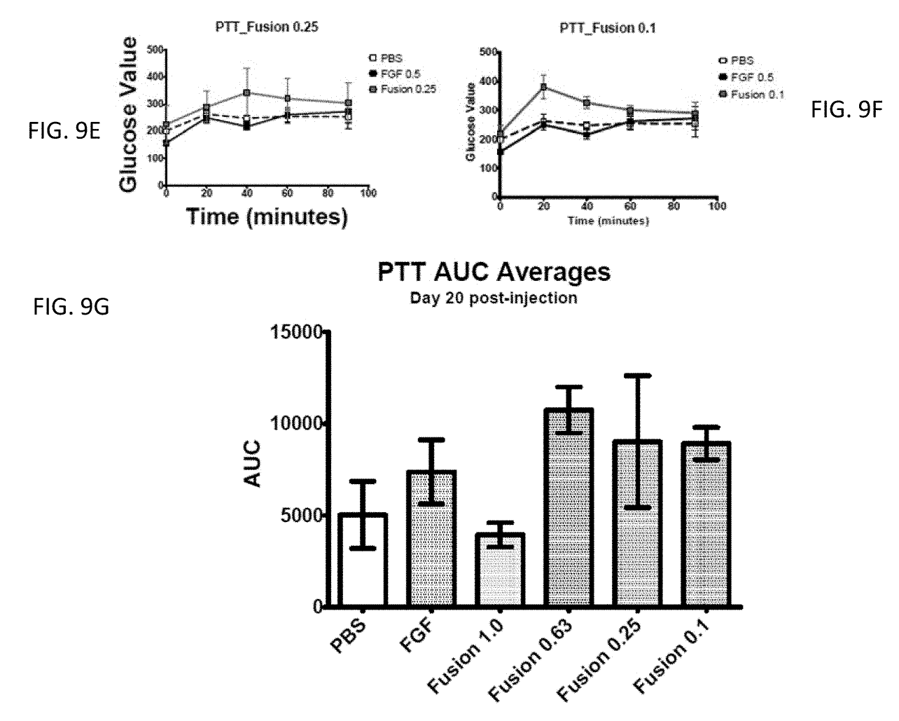

FIGS. 9A-9G show the effect of a single injection of PBS, FGF1, or FGF1-vagus targeting chimeric protein (fusion) on PTT 20 days after the injection, and after 16 hours of fasting. Single injections of FGF1 or FGF1-vagus targeting chimeric protein (fusion) do not lead to sustained changes in pyruvate tolerance tests. Thus, there is no long term change in the hepatic glucose production from treatment with either the FGF1 protein or FGF1-vagus targeting chimeric protein (fusion).

Based on these results, FGF1-vagus targeting chimeric proteins can be used to lower blood glucose in vivo for extended periods of time.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. An isolated protein, comprising:
a mutated mature fibroblast growth factor (FGF) 1 protein comprising at least 80%, at least 85%, at least 90%, at least 95%, at least 96%, at least 97%, at least 99%, or 100% sequence identity to SEQ ID NO: 420, 421, or 422, and retains the mutated amino acid(s) provided in the sequence, or
an FGF1-vagus targeting chimeric protein comprising an FGF1 protein and a vagus nerve targeting protein.

2. The isolated protein of claim 1, wherein the N-terminal amino acid is a methionine.

3. The isolated protein of claim 1, wherein the mutated mature FGF1 protein or the FGF1 protein of the FGF1-vagus targeting chimeric protein comprises a deletion of at least 9, at least 10, at least 11, at least 12, or at least 13 contiguous N-terminal amino acids from a native FGF1 protein.

4. The isolated protein of claim 1, wherein the mutated mature FGF1 protein or the FGF1 protein of the FGF1-vagus targeting chimeric protein comprises at least one point mutation shown in Table 1.

5. The isolated protein of claim 4, wherein the at least one point mutation comprises one or more of: Y8F, Y8V, Y8A, K9T, K9R, K9A, K10T , K12V, L14A, Y15F, Y15A, Y15V, C16V, C16A, C16T, C16S, S17R, S17K, N18R, N18K, H21Y, R35E, R35V, R35K, Q40P, Q43K, Q43E, Q43A, L44F, L46V, S471, S47A, S47V, E49D, E49K, E49Q, E49A, Y55F, Y55V, Y55S, Y55A, Y55W, A6, M67I, L73V, C83T, C83S, C83A C83V, E87V, E87A, E87S, E87T, E87Q, E87D, E87H, R88Y, R88L, R88D, H93G, H93A, Y94V, Y94F, Y94A, N95V, N95A, N95S, N95T, S99A, K101E, H102Y, H102A, A103G, Δ104-106, W107A, F108Y, V109L, L111I, K112D, K112E, K112Q, K113Q, K113E, K113D, N114K, N114R, S116R, C117V, C117P, C117T, C117S, C117A, K118N, K118E, K118V, R119G, R119V, R119E, Δ120-122, Q127R, Q127K, F132W, L133A, L133V, L133S, P134V, L135A, and L135S, wherein the numbering refers to the sequence shown SEQ ID NO: 5.

6. The isolated protein of claim 1, wherein the protein has
decreased mitogenicity compared to a native mature FGF1 protein;
increased blood glucose lowering ability compared to a native mature FGF1 protein; or
both.

7. The isolated protein of claim 1, wherein the FGF1 protein of the FGF1-vagus targeting chimeric protein comprises
at least 80%, at least 85%, at least 90%, at least 95%, at least 96%, at least 97%, at least 99%, or 100% sequence identity to SEQ ID NO: 5; or
at least 80%, at least 85%, at least 90%, at least 95%, at least 96%, at least 97%, at least 99%, or 100% sequence identity to any one of SEQ ID NOs: 10-422, and retains the retains the mutated amino acid(s) provided in the sequence.

8. The isolated protein of claim 1, wherein the vagus nerve targeting protein of the FGF1-vagus targeting chimeric protein comprises
at least 80%, at least 85%, at least 90%, at least 95%, at least 96%, at least 97%, at least 99%, or 100% sequence identity to SEQ ID NO: 423, 434, 435, 436, 437, or 438;
dulaglutide, liraglutide, lixisenatide, or albiglutide;
or combinations thereof.

9. The isolated protein of claim 1, wherein the FGF1-vagus targeting chimeric protein comprises a linker between the FGF1 protein and the vagus nerve targeting protein.

10. The isolated protein of claim 1, wherein the FGF1-vagus targeting chimeric protein comprises at least 80%, at least 85%, at least 90%, at least 95%, at least 96%, at least 97%, at least 99%, or 100% sequence identity to SEQ ID NO: 424, 425, 426, 427, 428, 429, 430, 431, 432, or 433 and retains the mutated amino acid(s) provided in the FGF1 protein of the FGF1-vagus targeting chimeric protein.

11. An isolated nucleic acid encoding the isolated protein of claim 1.

12. A nucleic acid vector comprising the isolated nucleic acid of claim 11.

13. A host cell comprising the nucleic acid vector of claim 12.

14. A method of reducing blood glucose in a mammal, comprising:
administering to the mammal a therapeutically effective amount of the isolated protein of claim 1, thereby reducing the blood glucose.

15. A method of treating a metabolic disease in a mammal, comprising:
administering to the mammal a therapeutically effective amount of the isolated protein of claim 1, treating the metabolic disease.

16. The method of claim 15, wherein the metabolic disease is type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), hyperlipidemia, hypertension, latent autoimmune diabetes (LAD), or maturity onset diabetes of the young (MODY).

17. The method of claim 14, wherein the method reduces fed and fasting blood glucose, improves insulin sensitivity and glucose tolerance, reduces systemic chronic inflammation, ameliorates hepatic steatosis in a mammal, reduces food intake, or combinations thereof.

18. The method of claim 14, wherein the therapeutically effective amount of the protein is at least 0.1 mg/kg.

19. The method of claim 14, wherein the administering is subcutaneous, intraperitoneal, intramuscular, intravenous or intrathecal.

20. The method of claim 14, wherein the mammal is a human, cat or dog.

21. The method of any of claim 14, wherein the method further comprises administering an additional therapeutic compound.

22. The method of claim 21, wherein the additional therapeutic compound is insulin, an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist.

23. The method of claim 22, wherein the PPAR-gamma agonist is a thiazolidinedione (TZD), aleglitazar, farglitazar, muraglitazar, or tesaglitazar.

24. The method of claim 23, wherein the TZD is pioglitazone, rosiglitazone, rivoglitazone, or troglitazone.
